(19) United States
(12) Patent Application Publication
(10) Pub. No.: US 2013/0165502 A1
(43) Pub. Date: Jun. 27, 2013

(54) Diagnostic, Prognostic and Therapeutic Uses of miRs in Adaptive Pathways and Disease Pathways

(71) Applicant: THE OHIO STATE UNIVERSITY, Columbus, OH (US)

(72) Inventors: Clay B. Marsh, Columbus, OH (US); Melissa G. Piper, Powell, OH (US); Duaa A. Dakhlallah, Columbus, OH (US)

(73) Assignee: THE OHIO STATE UNIVERSITY, Columbus, OH (US)

(21) Appl. No.: 13/711,253

(22) Filed: Dec. 11, 2012

Related U.S. Application Data
(63) Continuation-in-part of application No. 13/119,559, filed on Apr. 20, 2011.
(60) Provisional application No. 61/161,196, filed on Mar. 18, 2009.
(60) Provisional application No. 61/098,071, filed on Sep. 18, 2008.

(51) Int. Cl. A61K 31/7088; A61K 31/706; A61K 48/00

(52) U.S. Cl. CPC A61K 31/7088; A61K 31/706
USPC 514/44 R

(57) ABSTRACT

Described herein are methods and compositions for the diagnosis, prognosis and treatment of various adaptive and/or disease pathways by examining samples containing one or more miRs therein, and by formulating therapeutic agents therefrom.

### PRIORITY CLAIM AND STATEMENT REGARDING FEDERALLY SPONSORED RESEARCH

This application is a continuation-in-part application of U.S. Ser. No. 13/119,559 having a 35 U.S.C. §371 filing date of Apr. 20, 2011, which claims priority to PCT/US2009/057432 filed Sep. 18, 2009, which claims priority to U.S. Provisional Patent applications Ser. No. 61/098,071 filed Sep. 18, 2008 and Ser. No. 61/161,196 filed Mar. 18, 2009, which are fully incorporated herein by reference. This invention was not made with any government and the government has no rights in this invention.

### BACKGROUND OF THE INVENTION

Various conditions and/or diseases are characterized by injury (and, sometimes, subsequent tissue repair) that transiently or permanently results in changes in adaptive pathways and/or disease pathways. Non-limiting examples of adaptive pathways include one or more of: wound healing, post-surgical recovery, and trauma. Non-limiting examples of disease pathways include one or more of: organ fibrosis such as, but not limited to, cirrhosis, renal fibrosis and injury: solid organ cancer; bone marrow disorders; cardiac fibrosis/failure. A particular pathway is lung fibrosis, including idiopathic pulmonary fibrosis (IPF) associated disease or an interstitial lung disease (ILD).

Idiopathic pulmonary fibrosis (IPF) is an untreatable lung disease caused by repeated episodes of lung injury causing scarring of the lung and chronic inflammation that lead to irreversible thickening of air sacs wall in the lungs. There is no known cure and the progressive nature of this disease ultimately results in a dismal 5 yr mortality rate of 30-50%.

Idiopathic pulmonary fibrosis (IPF) represents the most aggressive form of interstitial lung disease (ILD) with a median survival of 3-5 years. Failure to resolve epithelial cell injury in the lung is critical to the pathogenesis of IPF. In addition, epithelial-mesenchymal transition (EMT), fibroblast proliferation and activation, and recruitment of inflammatory cells all contribute to extracellular matrix (ECM) accumulation in the lung.

MicroRNAs (miRNAs or miRs) are small single-stranded non-coding RNAs expressed in animals and plants. They regulate cellular function, cell survival, cell activation and cell differentiation during development. MicroRNAs regulate gene expression by hybridization to complementary sequences of target mRNAs resulting in either their inhibition of translation or degradation. MicroRNAs regulate gene expression by targeting messenger RNAs (mRNA) in a sequence specific manner, inducing translational repression or mRNA degradation, depending on the degree of complementarity between miRNAs and their targets.

The identification of one or more miRs which are differentially-expressed between normal cells and cells affected by IPF would be helpful. The present invention provides novel methods and compositions for the diagnosis, prognosis and treatment of IIPF.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### SUMMARY OF THE INVENTION

Described herein are methods of diagnosing or detecting susceptibility of a subject to one or more of a condition characterized by injury and tissue repair that transiently or permanently results in changes in one or more of an adaptive pathways and/or disease pathways.

Also described herein is a method of treating an inflammatory disorder in a subject having a decreased expression of DNA methyltransferase (DNMT), comprising administering to a subject an effective amount of at least one microRNA from the miR-17˜92 cluster, wherein the disorder has an decreased expression of a DNMT, as compared to a reference level.

In certain embodiments, the miRNA is selected from the group consisting of: miR-17-5p, miR-17-3p, miR-18, miR-19a, miR-19b, miR-20a and miR-92. In certain embodiments, the miR comprises miR-19b and/or miR-20a.

In certain embodiments, the method further comprises administering one or more additional pharmaceutical DNMT inhibitor compositions.

In certain embodiments, the additional DNMT inhibitor composition comprises 5′-aza-2′-deoxycytidine or an analog thereof.

In certain embodiments, the inflammatory disorder is idiopathic pulmonary fibrosis (IPF), systemic sclerosis, pulmonary fibrosis, liver fibrosis, kidney fibrosis, uterine fibrosis, vascular fibrosis including peripheral arterial disease, or interventional therapy triggered fibrosis. In certain embodiments, the subject is a human.

In certain embodiments, the method includes administering: a) ameliorates the fibrosis; b) slows further progression of the fibrosis; c) halts further progression of the fibrosis; and/or d) reduces the fibrosis.

In certain embodiments, the administration of the at least one miR decreases expression of DNMT in said idiopathic fibroblast cells without altering the phenotype of the non-idiopathic fibroblast cells.

Also described herein is a method of inhibiting an increase in DNTM levels induced by an inflammatory disorder over-expressing DNTM, comprising: contacting a human fibroblast cell over expressing human DNTM with an agent under conditions such that increases in DNTM in said fibroblast cell is inhibited, wherein said agent is an oligonucleotide that functions via RNA interference and the oligonucleotide sequence consists of at least one miR of the miR-17˜92 cluster.

Also described herein is a method of treating an inflammatory disorder in a subject having a decreased expression of DNA methyltransferase (DNMT), comprising administering to a subject an effective amount of 5′-aza-2′-deoxycytidine or an analog thereof.

Also described herein is a method of treating idiopathic pulmonary fibrosis, comprising administering to a patient in need thereof a therapeutically effective amount of microRNA selected from miR-17˜-92 cluster comprised of miR-17-5p, miR-17-3p, miR-18, miR-19a, miR-19b, miR-20a and miR-92 upregulator.

Also described herein is a method of treating an inflammatory disorder comprising: administering a therapeutically effective amount of at least one DNA-methyltransferase (DNMT) inhibitor composition to a subject in need thereof; and determining effectiveness of the DNMT inhibitor composition by measuring an increased expression of at least one microRNA selected from miR-17˜-92 cluster comprised of miR-17-5p, miR-17-3p, miR-18, miR-19a, miR-19b, miR-20a and miR-92.

In certain embodiments, the inflammatory disorder is idiopathic pulmonary fibrosis (IPF).

Also described herein is a method of assessing the effectiveness of an DNA-methyltransferase inhibitor composition on the treatment of a fibrotic disease, comprising: obtaining a biological sample from a subject before and after the treatment; selecting at least one miRNA whose level of expression is increased or decreased in a cell that is being effectively treated with the DNA-methyltransferase inhibitor composition, as compared to the level of expression in a cell that is not being effectively treated; measuring the level of the miRNA in the biological samples; and determining if the miRNA is present at an increased or decreased level in the biological sample obtained after the treatment as compared to the biological sample obtained either before the treatment or in a cell that is not being effectively treated; wherein an increased or decreased level of the miRNA indicates the effectiveness of the DNA-methyltransferase inhibitor composition in treating the disease.

In certain embodiments, the result of the miRNA assessment is used to optimize the dosing regimen of the subject.

In certain embodiments, the altered expression level is an increase in expression. In certain embodiments, the altered expression level is a decrease in expression.

In certain embodiments, the miRNA is selected from the miR-17˜92 cluster, and further wherein the expression is increased after treatment.

In certain embodiments, the treatment is a treatment for a fibrotic disorder.

In certain embodiments, the treatment is an aerosol administration of the DNA-methyltransferase inhibitor composition.

In certain embodiments, the subject is tested at a time interval selected from the group consisting of hourly, twice a day, daily, twice a week, weekly, twice a month, monthly, twice a year, yearly, and every other year.

Also described herein is a method of assessing the activity of a 5′-aza-2′-deoxycytidine type composition in a subject, comprising: obtaining a biological sample from the subject before and after treatment of the subject; and measuring the level of at least one miR selected from the miR-17˜92 cluster in the biological samples; wherein an increased level of one or more of the miRNAs indicates the activity of the composition.

In certain embodiments, the activity is the extent of the treatment by 5′-aza-2′-deoxycytidine.

In certain embodiments, the extent of the treatment is a dose administered or length of subject's exposure to 5′-aza-2′-deoxycytidine.

In certain embodiments, the treatment is a treatment for a fibrotic disease.

In certain embodiments, the treatment is an aerosol administration of 5′-aza-2′-deoxycytidine.

In certain embodiments, the subject is tested at a time interval selected from the group consisting of hourly, twice a day, daily, twice a week, weekly, twice a month, monthly, twice a year, yearly, and every other year.

Also described herein is a method of treating or delaying the onset or recurrence of a fibrotic associated disorder, wherein the disorder involves airway inflammation, fibrosis and excess mucus production, or at least one symptom thereof, the method comprising: administering an effective amount of: 5′-aza-2′-deoxycytidine and/or a miR-17˜92 gene product.

In certain embodiments, the 5′-aza-2′-deoxycytidine is administered by inhalation.

Also described herein is a composition comprising a pharmacologically effective dose of a 5′-aza-2′-deoxycytidine and a pharmacologically effective dose of one or more miR-17˜92 gene products.

In certain embodiments, the 5′-aza-2′-deoxycytidine and the one or more miR-17˜92 gene products are in dosage unit form.

In certain embodiments, the composition is in the form of a, spray or aerosol.

In certain embodiments, wherein the ratio of 5′-aza-2′-deoxycytidine to one or more miRNA selected from miR-17˜92 gene products in the dosage form is in the range of 2:1 to 1:2.

In certain embodiments, wherein the ratio of 5′-aza-2′-deoxycytidine to one or more miRNA selected from miR-17˜92 gene products in the dosage form is 1:2.

In certain embodiments, the composition further includes a pharmaceutically acceptable carrier.

In certain embodiments, the adaptive pathways include one or more of: wound healing, post-surgical recovery, and trauma.

Also, in certain embodiments, the disease pathways include one or more of: organ fibrosis such as, but not limited to, cirrhosis, renal fibrosis and injury: solid organ cancer; bone marrow disorders; cardiac fibrosis/failure.

In a particular embodiment, the disease pathway comprises lung fibrosis, including idiopathic pulmonary fibrosis (IPF) associated disease or an interstitial lung disease (ILD).

In another broad aspect, there is provided herein a method of diagnosing or detecting susceptibility of a subject to one or more of an idiopathic pulmonary fibrosis (IPF) associated disease or an interstitial lung disease (ILD), comprising: determining the level of at least one miR gene product in the miR-17˜92 cluster in a sample from the subject; and comparing the level of at least one miR gene product in the sample to a control, wherein an alteration in the level of the at least one miR gene product in the sample from the subject, relative to that of the control, is diagnostic or prognostic of such disease.

In certain embodiments, the miR gene product includes one or more of: miR-17-3p, miR-17-5p, miR-18a, miR-19b and miR-20a.

In certain embodiments, the miR gene product comprises one or more of miR-19a, miR-19b and miR-20a.

In certain embodiments, one or more of the miRs are expressed at low levels in an IPF sample.

In certain embodiments, the control is selected one or more of: a reference standard; the level of the at least one miR gene product from a subject that does not have the disease; and the level of the at least one miR gene product from a sample of the subject that does not exhibit such disease.

In certain embodiments, the subject is a human. In certain embodiments, the alteration is an increase in the level of at least one miR gene product in the sample. In certain embodiments, the alteration is a decrease in the level of at least one miR gene product in the sample.

In another broad aspect, there is provided herein a method of inhibiting progression or proliferation of an idiopathic pulmonary fibrosis associated disorder in a subject, comprising: i) introducing into at least one cell of the subject one or more agents which alter expression and/or activity of at least one miR in the miR-17˜92 cluster within the cell, and ii) maintaining the cells under conditions in which the one or more agents: inhibits expression or activity of the miR; enhances expression or activity of one or more target genes of the miR; or, results in a combination thereof, thereby inhibiting progression or proliferation of the disease or disorder. In certain embodiments, the cell is a human cell.

In another broad aspect, there is provided herein a method of identifying a therapeutic idiopathic pulmonary fibrosis (IPF) agent, comprising: providing a test agent to a cell and measuring the level of at least one miR in the miR-17˜92 cluster associated with an altered expression levels in the cells, wherein an alteration in the level of the miR in the cell, relative to a suitable control cell, is indicative of the test agent being a therapeutic agent.

In another broad aspect, there is provided herein a method for regulating levels of one or more proteins in a subject having, or at risk of developing, an idiopathic pulmonary fibrosis (IPF) associated disorder, comprising: altering the expression of at least one miR gene product in the miR-17-92 cluster lung cells in the subject.

In certain embodiments, at least one protein comprises: c-myc, CTGF, TSP1, HDAC4.

In certain embodiments, the method includes altering expression of one or more of: miR-19a, miR-19b, and miR-20a.

In certain embodiments, the subject has idiopathic pulmonary fibrosis (IPF).

In certain embodiments, the subject has an interstitial lung disease (ILD).

In another broad aspect, there is provided herein a method for assessing prognosis in a subject with an idiopathic pulmonary fibrosis associated disorder, comprising: determining a level of at least one miR in the miR-17˜92 cluster which alters expression of one or more of the protein levels of for c-myc, CTGF and HDAC4 as a prognostic indicator of disease progression.

In certain embodiments, at least miR-19b is used be a prognostic indicator of disease state.

In another broad aspect, there is provided herein a method for assessing prognosis in a subject with an idiopathic pulmonary fibrosis associated disorder, comprising: determining an altered expression of one or more of the protein levels as a prognostic indicator of disease progression, wherein at least miR-19b and mir-20a are down regulated with increasing severity of disease in patients with IPF.

In another broad aspect, there is provided herein a method for altering the expression of a target gene in a subject having, or at risk or developing idiopathic pulmonary fibrosis (IPF), comprising: inducing expression of one or more miRs in the miR-17˜92 clusters in cells in the subject.

In certain embodiments, the method includes inducing expression by transient transfection in IPF fibroblast cells in the subject sufficient to alter expression of at least one target and/or to change at least one gene networks, to expression those present in normal fibroblast cells.

In certain embodiments, one or more miRs of the miR-17˜92 cluster downregulate expression of one or more genes selected from: CTGF, TGFβ, MMPs, VEGF and thrombospondin-1 (TSP1).

In certain embodiments, the method includes forcing expression of the miR-17˜92 cluster sufficient to downregulate the expression of one or more of the genes and sufficient to down-regulate the signaling networks associated therewith.

In another broad aspect, there is provided herein a method for treating idiopathic pulmonary fibrosis (IPF) fibroblasts in lung cells in a subject, comprising introducing one or more miRs in the miR-17˜92 cluster into the cells in an amount sufficient to recover a proliferative and younger phenotype in the cells.

In another broad aspect, there is provided herein a method for enhancing wound healing in lung cells a subject having or at risk of developing idiopathic pulmonary fibrosis (IPF), comprising: transfecting the lung cells with one or more miRs in the miR-17˜92 cluster.

In another broad aspect, there is provided herein a method for treating lung fibroblast cells a subject having or at risk of developing idiopathic pulmonary fibrosis (IPF), comprising: transfecting the fibroblast cells with one or more miRs in the miR-17˜92 cluster members in an amount sufficient for: i) at least certain of the cells to assume a phenotype similar to non-IPF fibroblast cells; and/or ii) a subsequent increase in expression of one or more proteins selected from: CTGF, TSP-1, MMPs, TGF-beta and VEGF.

In another broad aspect, there is provided herein a method for increasing lung cell development in a subject in need thereof, comprising increasing expression of one or more miRs in the miR-17˜92 cluster in lung cells of the subject.

In another broad aspect, there is provided herein a method for enhancing lung tissue repair and remodeling in response to lung injury in a subject, comprising increasing expression of one or more miRs in the miR17˜92 cluster in lung cells in the subject.

In another broad aspect, there is provided herein a method for treating human idiopathic pulmonary fibrosis (IPF) tissue, comprising increasing expression of one or more miRs in the miR17˜92 cluster in cells in the tissue.

In another broad aspect, there is provided herein a method for altering expansion of marrow precursor cells after lung injury in a subject, comprising increasing expression of one or more miRs in the miR17˜92 cluster in lung cells in the subject.

In certain embodiments, the method of any one of the treatment claims includes the use of miR-19b as the miR selected from the miR-17˜92 cluster.

In certain embodiments, at least one other miR in the miR-17˜92 cluster is used in combination with miR-19b for therapeutic impact.

In another broad aspect, there is provided herein a method for detecting changes in myofibroblast production and/or detecting alterations in epithelial cell-to-mesenchymal cell transition in a subject having, or at risk of developing idiopathic pulmonary fibrosis (IF), comprising: measuring levels of one or more miRs in the miR17˜92 cluster in lung cells in the subject.

In certain embodiments, at least one of miR-19b and miR-20a are down regulated with increasing severity of disease in patients with IPF.

In another broad aspect, there is described herein method of detecting susceptibility of a subject to an idiopathic pulmonary fibrosis (IPF) associated disease, comprising: i) determining the level of at least one miR gene product selected from the miR-17˜92 cluster in a sample from the subject; and ii) comparing the level of at least one miR gene product in the sample to a control, wherein an increase in the level of the at least one miR gene product in the sample from the subject, relative to that of the control, is diagnostic or prognostic of such disorder.

In certain embodiments, the control may be one or more of: a reference standard; the level of the at least one miR gene product from a subject that does not have the disorder; and iii) the level of the at least one miR gene product from a sample of the subject that does not exhibit such disorder. In certain embodiments, the subject is a human. In a particular embodiment, the alteration is a decrease in the level of the miR gene product in the sample.

Various objects and advantages of this invention will become apparent to those skilled in the art from the following detailed description of the preferred embodiment, when read in light of the accompanying drawings.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIG. 1—Table showing upregulated miRs in human interstitial lung disease (ILD), as compared to normal.

FIG. 2—Table showing downregulated miRs in human interstitial lung disease (ILD), as compared to normal.

FIG. 3—Hierarchical cluster analysis of miRs in lung tissue from patients with interstitial lung disease ((ILD) and normal tissue (CTRL).

FIG. 4—Graph showing the validation of expression of miR-19b in ILD tissue, showing percent of forced vital capacity (FVC).

FIGS. 5A-5C—Graphs showing the validation of miR expression in human idiopathic pulmonary fibrosis (IPF) v. control (CTRL) by quantitative RT-PCR: FIG. 5A showing miR-17-5p, miR173p, miR18a, miR-19a, miR-19b, miR-20a and miR-92; FIG. 5B showing miR-29a, miR-29b and miR29c; and, FIG. 5C showing miR-34a, miR-34b and miR-34c.

FIG. 6—Graph showing miR-17˜92 expression in normal human lung fibroblast and IPF human fibroblast.

FIG. 7—Comparison between normal lung fibroblast (left column) and IPF lung fibroblast (right column) for: Ets-2, TGFβ, Elk3, E2F1, CTGF, Tsp-1 and β-actin.

FIG. 8—Graph showing the miR-17˜92 cluster expression in human IPF samples.

FIG. 9—Table showing microRNAs involved in regulating gene expression involved in IPF.

FIGS. 10A-10B—Hierarchical clustering of gene expression profiles from IPF/ILD, COPD, and control (CTRL) samples. All tissue samples were obtained from the LTRC or CHTN. RNA was isolated and profiled by Affymetrix gene chips: FIG. 10A—Unsupervised clustering of mRNA profiles from 21 patients with IPF/ILD, 6 patients with COPD, and 5 controls (uninvolved lung tissue from patients undergoing surgery for lung cancer). The unsupervised clustering was applied to the gene expression profiles after a one-way ANOVA test. The program, Bioconductor, was used for this analysis; and, FIG. 10B-IPF/ILD profiles clustered with themselves after a 2-way ANOVA test. The FVC group ILD-1 (<50% FVC), ILD-2 (50-80% FVC), or ILD-3 (>80% FVC, least severe breathing impairment) is shown as the last digit of the sample identification. At least some of the IPF/ILD patients falling into groups 1, 2, or 3 clustered together by this analysis.

FIGS. 10C-10D—IPF/ILD patients with distinct forced vital capacity have different patterns of gene expression: Increased expression of VEGF (FIG. 10C) and CTGF (FIG. 10D) according to disease severity. Real-time PCR reaction was performed for VEGF and CTGF. Shown is the average relative expression normalized to 18s internal control±S.E.M (Control n=3, >80% FVC n=4, 50-80% FVC n=4, and <50% FVC n=4).

FIG. 10E—Table showing biological pathways implicated in ILD: preliminary comparison of ILD profiles relative to control profiles. The mean expression value for each gene within a sample grouping (IPF/ILD or CTRL) was fit into an analysis of variance model. Confidence intervals were calculated across all results using Tukey's Honest Significant Differences calculation in R/Bioconductor, producing an adjusted p-value. The 10 pathways with the highest significance are shown.

FIGS. 11A-11C—Graphs showing the decreased expression of the miR-17˜92 cluster in lung tissue from FVBM mice treated with bleomycin (Bleo), as compared to vehicle: FIG. 11A showing miR-19b, miR-20 and miR-92a; FIG. 11B showing miR17-5p and miR-19a; and, FIG. 11C showing miR17-3p and miR-18a.

FIGS. 11D-11E—Pathological (FIG. 11D) and protein (FIG. 11E) assessment of bleomycin-induced fibrosis in mice for PBS and Bleomycin.

FIGS. 12A-12B—Graphs showing changes in expression of the miR-17˜92 cluster in bleomycin-induced fibrosis in C57BL/6 mice, as compared with PBS samples.

FIG. 13—Graph showing IPF gene expression in bleomycin treated C67BL/6 mice.

FIGS. 14A-14K—graphs showing the effect of over-expression of miR-17˜92 cluster on IPF gene expression for: left-to-right: Untreated normal lung fibroblast; Normal+MiR-17-92 cluster (0.5 ug); Normal+miR-17˜92 cluster (1.0 ug); Untreated IPF lung fibroblast; IPF+miR-17˜91 cluster (0.5 ug); IPF+miR-17˜92 cluster (1.0 ug): FIG. 14A=Tsp-1, FIG. 14B=VEGF, FIG. 14C=Elk3, FIG. 14D=HIF1A, FIG. 14E=TN-C, FIG. 14F=HIF1B, FIG. 14G=Ets-2, FIG. 14H=Ets-1, FIG. 14I=CTGF, FIG. 14J=Col13a, and FIG. 14K=Col1a.

FIGS. 15A-15B—Graphs showing fold change in expression for re-introduction of the miR-17-92 cluster in IPF-derived lung fibroblasts decreases expression of VEGF and CTGF. Cells were transfected with either empty vector (pcDNA3.1) or the pcDNA3.1/miR-17˜92 expression vector using Effectene then cultured for 48 h. RNA was isolated and then subjected to qRT-PCR using specific primers to VEGF (FIG. 15A) or (B) CTGF (FIG. 15B) and 18s was used as an internal control. Fold change compared to untransfected cells was determined Data shown is the average±SEM (n=3).

FIG. 16—Photographs showing miR-17˜92 transfection induces phenotypic changes in lung fibroblasts derived from patients with IPF. IPF-derived lung fibroblasts were transfected with the miR-17˜92 cluster. Equal cell numbers for untransfected (IPF) and transfected (IPF+17-92 cluster) cells were cultured and photographed daily to visualize phenotypic changes.

FIGS. 17A-17T—Graphs for gene expression in human IPF patient samples based on disease severity; left-to-right: Normal (n=3); group 3>80% (n=4); group 2<50-80% (n=4); group 1<50%, showing the Relative Expression 2̂(−dCT): FIG. 17A=IL-6; FIG. 17B=Map3k; FIG. 17C=Mmp-7; FIG. 17D=SOCS-3; FIG. 17E=FAS; FIG. 17F=FN-1; FIG. 17G=TSC-2; FIG. 17H=SOX-17; FIG. 17I=THB-1; FIG. 17J=IL-1-R2; FIG. 17K=Ets-2; FIG. 17L=Ets-2; FIG. 17M=Col-1; FIG. 17N=Elk-3; FIG. 17O=Tert; FIG. 17P=Col-3; FIG. 17Q=Col-13a; FIG. 17R=LBTP; FIG. 17S=CTGF; FIG. 17T=VEGF.

FIG. 18-Protein expression in lung tissue from patients with IPF for TPS-1. CTGF, Ets-2, TGFβ and Elk3.

FIGS. 19A-19B—Photographs showing miR19b expression in human lung tissue: FIG. 19A=normal lung tissue (left) and IPF (group 2, 50-80% FVC) (right); FIG. 19B=IPF (group 3, >80% FVC) (left), and IPF (group1, <50% FVC) (right).

FIG. 20A—Photograph showing miR-20a expression in human lung tissue: IPF (left) and normal (right).

FIG. 20B—Photograph showing let-7 expression in human lung tissue: IPF (left) and normal (right).

FIGS. 21A-21D—Graphs showing the miRNA expression in human lung fibroblast cell lines.

FIG. 22-Protein expression in human normal and IPF lung fibroblast cell lines, where N=Normal, and I=IPF.

FIGS. 23A-23B—Photographs showing morphology for human lung fibroblasts in Normal (FIG. 23A) and IPF (FIG. 23B).

FIGS. 24A-24B—Photographs showing IPF-derived fibroblasts transfected with the miR-17-92 cluster begin to assume a phenotype similar to normal lung fibroblasts: FIG. 24A—IPF lung fibroblast, untreated; FIG. 24B (left)=IPF lung fibroblast, +0.5 μg miR-17˜92 cluster; and, FIG. 24B (right)=IPF lung fibroblast, +1 μg miR-17˜92 cluster.

FIGS. 25A-25B—Photographs showing over-expression of the miR-17˜92 cluster in normal lung fibroblasts does not alter their phenotype: FIG. 25A—normal lung fibroblast, untreated; FIG. 25B (left)=normal lung fibroblast, +0.5 μg miR-17˜92 cluster; and, FIG. 25B (right)=normal lung fibroblast, +1 μg miR-17˜92 cluster.

FIGS. 26A-26B—Photographs showing over-expression (FIG. 26B), but not knockdown expression (FIG. 26A), of miR-19b (left) or miR-20a (right), induces phenotypic changes in IPF lung fibroblast cell lines.

FIGS. 27A-27B—Photographs showing knockdown expression (FIG. 27A) of miR-19b (left) or miR-20a (right) induces normal lung fibroblast cell lines to become phenotypically similar to the IPF lung fibroblast cell lines, as compared to over-expression (FIG. 27B).

FIG. 28A—Graph showing relative miR-19b expression in human fibroblast (CCL-204): untransfected; over-expressed; and knockdown (KO).

FIG. 28B—Graph showing relative miR-20ab expression in human fibroblast (CCL-134): untransfected; over-expressed; and knockdown (KO).

FIG. 28C—Graph showing relative miR-20a expression in human fibroblast (CCL-204): untransfected; over-expressed; and knockdown (KO).

FIG. 28D—Graph showing relative miR-19b expression in human fibroblast (CCL-134): untransfected; over-expressed; and knockdown (KO).

FIGS. 29A-29J—Graphs showing increased gene expression in both normal and IPF fibroblast cell lines when expression of either miR-19b or miR20a is knockdown.

FIG. 30-Decreased protein expression following transfection of miR-17˜92 in IPF lung fibroblast cell lines was found, where U=Untransfected, M=Mock transfection, V=Empty vector, and C=miR-17˜92 cluster.

FIG. 31-Decrease protein expression following transfection of either miR-19b or 20a in IPF lung fibroblast cell lines, where U=untransfected, Sc=scramble control, +20=miR-20a, 20=miR-20a antagomirs, +19=miR-19b, and 19=miR-19b antagomirs.

FIG. 32—Graph showing location of CpG islands in the promoter of miR-17˜92 and primer sequences used for DNA methylation studies.

FIG. 33—Graph showing percent DNA Methylation for normal and IPF in fibroblast cell lines and in primary human tissue.

FIGS. 34A-34B—Data showing decreased expression of the miR-17˜92 cluster in human IPF:

FIG. 34A—Graph showing expression of each miRNA from the miR-17˜92 cluster was determined by qRT-PCR from control (n=10), >80% FVC (n=7), 50-80% FVC (n=8) and <50% FVC (n=9) lung tissue samples. Data were normalized to miR-191. Relative copy number (RCN, 2̂-dCT*100) was determined Data are expressed as the average RCN±S.D., *p<0.018 compared to control tissue. Comparison of the mild disease (80% FVC) to control tissue for miRs-19b and -92, p=0.1325 and 0.1320, respectively.

FIG. 34B—Photographs showing in situ hybridization, performed using LNA-modified DNA probes for let-7c (positive control), and miR5-19b and -20a at a magnification of 400×. Scrambled probes were used as a negative control. The arrows denote positively stained cells (dark blue/purple). Shown are representative images (n=3 per group).

FIGS. 35A-35C—Data showing introduction of miR-17˜92 in IPF lung fibroblasts induces phenotypic and molecular changes. Normal and IPF lung fibroblasts were transfected with an expression vector containing the miR-17˜92 cluster. Empty vector-transfected cells (vector) also served as a negative control:

FIG. 35A—Photographs showing after 24 hrs, cells were stained with phalloidin and DAPI 0.5 μg/ml then photographed using an Olympus inverted fluorescent at 10× magnification. Shown are representative images from three independent experiments.

FIG. 35B—Graphs showing results where the red fluorescence of phalloidin was quantitated and indicated as a percentage compared to cell number as enumerated by DAPI positive nuclei. Shown is the average intensity±S.D. Significant decrease was apparent in miR-17˜92 transfected cells compared to vector only transfected IPF cells, *p=0.0327; differences in phalloidin staining for IPF vector-transfected IPF cells compared to untransfected (p=0.2549) or vector-transfected (p=0.3534) normal lung fibroblasts.

FIG. 35C Graphs showing results where RNA was isolated and qRT-PCR was performed for the indicated genes. Data were normalized using GAPDH as a housekeeping control. Data are expressed as the average relative copy number (RCN)±S.D. from three experiments. Comparison was made between vector only transfected IPF cells to the miR-17˜92 transfected IPF cells, *p<0.004.

FIGS. 36A-36B—Graphs showing where increased DNA methylation of the miR-17˜92 promoter in IPF:

FIG. 36A—DNA was isolated from control lung tissue (n=3) and individuals with IPF (n=3 per severity group). Samples were selected from the cohort used in FIG. 34. Data are presented as the average percent of unmethylated or methylated DNA of the miR-17˜92 promoter±S.D. Statistical difference was determined by comparing the % unmethylated to % methylated for each severity group, *p<0.003.

FIG. 36B—DNA was isolated from normal or IPF fibroblasts. DNA methylation for the miR-17˜92 promoter was determined. Shown is the percent DNA methylation, n=6 (*p<0.0001 normal fibroblast compared to IPF fibroblast for unmethylated or methylated DNA).

FIGS. 37A-37B—Graphs showing: treatment of IPF lung fibroblasts with 5′-aza-2′-deoxycytidine liberates miRNA expression and downregulates target mRNAs. IPF lung fibroblast cell line was either treated with the vehicle control DMSO or treated with 0.5 PM 5′-aza-2′-deoxycytidine (5′-Aza) for 24 hrs. RNA was isolated and subjected to qRT-PCR analysis for (FIG. 37A) miRNA or (FIG. 37B) fibrotic genes. Data were normalized to (FIG. 37A) RNU48 or (FIG. 37B) CAP-1 expression. Shown is the average relative copy number (RCN)rS.D. from two independent experiments.

FIGS. 38A-38C—Graphs showing DNMT-1 is altered in IPF and a target of the miR-17˜92 cluster:

FIG. 38A—Graph showing DNMT-1 expression was examined by qRT-PCR from a select cohort of tissue samples shown in FIG. 34 (n=3, per each disease severity group and control tissue samples). Using GAPDH as an endogenous control, the average relative copy number (RCN)±S.D. was calculated. Significance *p<0.05 is shown.

FIG. 38B—Graph showing IPF lung fibroblasts cells were transfected with or without miR-19b or 20a antagomir to knock-down (KD) their expression. Following treatment with 5′-aza-2′-deoxycytidine (5′ Aza) for 24 hrs, RNA was extracted and DNMT-1 expression quantitated by qRT-PCR. DNMT-1 expression was normalized to GAPDH in vehicle-treated untransfected cells. Shown is the average fold-change r S.D. (n=3). Significance for 5′-aza-2′-deoxycytidine treated cells compared to 5′-aza-2′-deoxycytidine/miR-19b KD cells is *p<0.001. No clinical difference was apparent between 5′-aza-2′-deoxycytidine treated cells and 5′-aza-2′-deoxycytidine/miR-20a KD cells.

FIG. 38C—Graph showing where the wild-type (WT) or mutated (mut) 3′UTR for DNMT-1 was cloned into the pGL-3 Firefly luciferase vector and transfected in the presence of the indicated miRNAs in HEK 293 cells. Irrelevant scrambled miRNA served as a control. Cells were also co-transfected with the Renilla luciferase construct, pRL-TK. Luciferase production was measured first for the Firefly luciferase followed by the Renilla luciferase from the culture supernatant after 24 hrs. Firefly luciferase was normalized to the Renilla luciferase. Shown is the average luciferase production from 8 independent experiments (r S.D.). WT DNMT-1 was compared to the mutated DNMT-1 for each specified miRNA, * p<0.001.

FIG. 39A-39C—Data showing in vivo treatment of mice with 5′-aza-2′-deoxycytidine attenuates bleomycin-induced fibrotic gene expression. Mice were treated with 0.035 U/kg twice weekly for 4 weeks with bleomycin (n=4) or PBS alone (n=4). A set of bleomycin treated mice were injected with 0.156 mg/kg/week of 5′-aza-2′-deoxycytidine i.p. for the last 2 weeks of bleomycin treatment (n=4) and designated as Bleomycin (4 wk) 5′ Aza (2 wk). As a control, mice were treated with PBS alone for 4 weeks or PBS for 2 weeks and then 5′-aza-2′-deoxycytidine for 2 weeks without bleomycin, [5′Aza (2wk)]:

FIG. 39A—Graph showing results where RNA was extracted from the lung tissue and miRNA expression was examined by qRTPCR and normalized using snoRNA-202 expression. Shown is the average relative copy number (RCN)rS.D., * p value <0.001 compared to bleomycin only treated mice.

FIG. 39B—Photographs showing where lung tissue was section and paraffin-embedded then subjected to trichrome staining to detect collagen deposition. The larger image is at 10× magnification while the inset is a 4× magnification. Shown are representative images from a mouse from each group.

FIG. 39C—Graph showing results where trichrome sections were blindly assessed by a board certified pathologist. The average arbitrary score rS.D. is shown (n=4 per group). Significance compared to bleomycin only treated mice, * p<0.0075 and ** p=0.1348.

FIG. 39D—Graph showing results where RNA was subjected to qRT-PCR for the indicated genes as well as (E) DNMT-1 expression. CAP-1 served as an endogenous control for normalization for fibrotic genes and DNMT-1. Data are expressed as the relative copy number (RCN)rS.D., * p value <0.001 compared to bleomycin-treated mice.

FIG. 39E—Graph showing results where DNA were isolated and subjected to analysis for promoter DNA methylation of the miR-17˜92 cluster. Data presented is the average % unmethylated and % methylated, n=3 mice per treatment group. * p value <0.001 compared to bleomycin only treated mice.

FIGS. 40A-40B—Graphs showing increased fibrotic gene expression in IPF:

FIG. 40A—Graphs showing results where fibrotic gene expression was confirmed by qRT-PCR in lung tissue from patients with IPF and control tissue. Relative copy number (RCN, 2A-dCT*100) was determined using GAPDH as an endogenous control. Data is expressed as the average RCN±S.D, *p<0.005.

FIG. 40B—Graph showing expression of the miRNAs contained in the miR17-92 cluster was determined in lung tissue from patients with COPD. Data was normalized using the average the Ct value for miR-191 and RNU6. Shown is the average fold ±S.D change in expression of COPD tissue samples compared to control tissue samples (n=11). Tissue was stratified according to FVC; FVC <50% and FVC 50-80%, n=5 per each severity group. Significance is indicated as *p<0.0014, ** p<0.05 and #p=0.0617.

FIGS. 41A-41B—Photographs showing results of co-expression analysis for miR-19b within control lung tissue. Serial control tissue sections used in FIG. 34B were stained for miR-19b and (FIG. 41A) CD45 or (FIG. 41B) epithelial cytokeratin AE1/3. CD45 was converted to red fluorescence while AE1/3 was converted to green:

FIG. 41A—Photographs where the colorimetric image is shown in the lower right panel for CD45 (brown) and miR-19 (blue). A strong signal for miR-19 is apparent in the bronchiole epithelium (arrow). The Nuance system converted these signals to blue fluorescence for miR-19b (upper left) and red fluorescence red for CD45 (upper right). Mixing of the two signals (lower left) shows that the miR-19b positive cells do not express CD45.

FIG. 41B—Photographs where the lower right panel shows the calorimetric based image for miR-19b (blue) and the epithelial cytokeratin AE1/3 (brown). The Nuance conversion is shown in upper left panel for miR19b (blue) and AE1/3 (green, upper right panel). Overlay of the two signals (lower left) shows that most of the miR-19b positive cells do co-express the cytokeratin in both the bronchiole (bronchial epithelium) and alveolar lining (alveolar pneumocytes) as seen by the fluorescent yellow.

FIGS. 42A-42B—Data showing the assessment of a Smooth Muscle Actin (a-SMA) in human lung fibrotic tissue—Immunohistochemistry (FIG. 42A) and quantification (FIG. 42B) of the staining for microscope high power field (HPF) (average ±S.D., n=2) for a-SMA (red staining) is shown for the slides used in FIG. 34B. Despite increase in the a-SMA staining in the 50-80% FVC (moderate), similar expression pattern demonstrating the presence of myofibroblasts in areas of fibrotic tissue is apparent for the 5080% FVC (moderate) and <50% FVC (severe) groups.

FIGS. 43A-43D—Data showing expression of the miR-17-92 cluster in lung fibroblasts and epithelial cells:

FIG. 43A—Graph showing results where lung epithelial and fibroblasts were isolated from untreated mice and miRNA expression was examined by qRT-PCR. Shown is normalized expression to snoRNA 202 as relative copy number (RCN) expressed as 2̂-dCT*100±S.D., n=5.

FIG. 43B—Graph showing results where human lung fibroblast cell lines from a normal individual or from a patient with IPF were obtained from ATCC. Cells were either left untransfected or transfected with the miR-17-92 cluster. RNA was extracted and subjected to qRT-PCR for each miRNA from the miR-17-92 cluster. The miRNA expression was normalized using miR-191 as an endogenous control. Shown is the relative copy number (RCN) expressed as 2̂-dCT*100±S.D, n=2.

FIG. 43C—Graph showing results where Col13a1 is directly regulated by miRNAs encoded by the miR-17-92 cluster. As expected, a significant decrease (average ±S.D., n=4) in luciferase production was apparent in the presence of the wild-type (WT) 3′UTR compared to the mutated (mut) 3′UTR, *p<0.0005 and #p=0.0370.

FIG. 43D—Graph showing results where average RCN (±S.D.) for HIFI-a in untransfected and miR-17—92 transfected normal and IPF cells (n=3). The RCN was determined using GAPDH as an endogenous control.

FIGS. 44A-44C—Data showing miR-17˜92 is epigenetically altered in IPF fibroblast cell line and modification results in phenotypic changes and reduction in DNMT-1 expression:

FIG. 44A—Photographs showing where IPF fibroblast cells were treated with 0.5 μM 5′-aza-2′-deoxycytidine (5′-Aza) for 24 hrs and stained with rhodamine-conjugated phalloidin then photographed. Shown is a representative image from three independent experiments.

FIG. 44B—Graph showing results where the fluorescence intensity for phallodin was quantitated per cell using DAPI staining as reference. Shown is the average ±S.D. Comparison of 5′-Aza-treated cells to PBS control, *p=0.0137.

FIG. 44C—Graph showing results where cells were transfected with either an expression containing the miR-17-92 cluster or an empty vector (Vector) with either 0.5 or 1.0 pg of DNA. Cells subjected to a mock transfection without DNA served as an additional negative control. After 24 hrs, RNA was isolated from the cells and subjected to qRT-PCR to determine DNMT-1 expression. RNU38B served as an endogenous control. The average relative copy number (RCN)±S.D. from three independent experiments is shown. Statistical difference was determined comparing samples to vector control, *p<0.05.

FIGS. 45A-45C—Graph showing results where DNA methylation of the promoter of fibrotic genes. Shown is the DNA methylation status of fibrotic genes, Col13a, Col1a, CTGF and VEGF for (FIG. 45A) human lung tissue samples, (FIG. 45B) human lung fibrosis cell lines and (FIG. 45C) mice treated with or without bleomycin. Shown is the average ±S.D.

FIG. 46—Photograph showing full size image of miRNA in situ hybridization with INC staining Shown is the full size image of the miRNA staining from FIG. 34B. Serial sections from FIG. 34B were stained with keratin, a-SMA and CD45. The Nuance system (lower panel) converted the signals for a-SMA (brown fluorescence) and CD45 (red fluorescence).

FIG. 47—Photographs showing full size image of phalloidin staining of miR-9792 transfected cells. Shown is the full size image of the phalloidin staining from FIG. 35A.

### DETAILED DESCRIPTION OF THE PREFERRED EMBODIMENT

The present invention is based, in part, on the identification of specific microRNAs (miRNAs) that are involved in an inflammatory response and/or have altered expression levels. The invention is further based, in part, on association of these miRNAs with particular diagnostic, prognostic and therapeutic features.

In a broad aspect, described herein are methods regarding the introduction into cells of one or more nucleic acids that function like miRNA or inhibit the activities of one or more miRNAs in cells. The invention concerns nucleic acids that perform the activities of endogenous miRNAs when introduced into cells. In certain embodiments, these nucleic acids can be synthetic miRNA.

Also described herein are methods of characterizing an miRNA activity or function in a cell. In some embodiments, a method comprises: a) introducing into one or more cells a synthetic miRNA molecule; and b) comparing one or more characteristics of cell(s) having the RNA molecule with cells in which the synthetic miRNA molecule has not been introduced. In certain embodiments, the cells with the synthetic miRNA may be compared to cells in which a different molecule was introduced (such as a negative control that does not include an miRNA region or has an miRNA region for a different miRNA). It is to be understood that the compared cells need not be evaluated at the same time; the comparison cells need not have been cultured at the same time; and/or one may refer to a report or previous observation.

Also described herein are methods which include reducing or eliminating activity of one or more miRNAs from a cell comprising: a) introducing into a cell an miRNA inhibitor. In certain embodiment, methods also include comparing one or more characteristics of a cell having the miRNA inhibitor with a cell not having the miRNA inhibitor.

In some embodiments, it may be useful to know whether a cell expresses a particular miRNA endogenously or whether such expression is affected under particular conditions or when it is in a particular disease state. Thus, in some embodiments, methods include assaying the cell for the presence of the miRNA that is effectively being introduced by the synthetic miRNA molecule or inhibited by an miRNA inhibitor. In some embodiments, methods include a step of generating an miRNA profile for a sample. The term “miRNA profile” generally refers to a set of data regarding the expression pattern for a plurality of miRNAs in the sample; it is contemplated that the miRNA profile can be obtained using an miRNA array. In some embodiments of the invention, an miRNA profile can be generated by steps that include: a) labeling miRNA in the sample; b) hybridizing the miRNA to an miRNA array; and, c) determining miRNA hybridization to the array, wherein an miRNA profile is generated.

Additionally, a cell that is introduced with a synthetic miRNA or an miRNA inhibitor may be subsequently evaluated or assayed for the amount of endogenous or exogenous miRNA or miRNA inhibitor. In additional embodiments, the synthetic nucleic acid can be introduced into the cell by any suitable method, including but not limited to calcium phosphate transfection, lipid transfection, electroporation, microinjection, or injection. In addition, a cell may be in a subject, which may be a patient or an animal model. In this case, synthetic nucleic acids can be administered to the subject or patient using modes of administration that are well known to those of skill in the art, particularly for therapeutic applications. It is particularly contemplated that a patient is human or any other mammal or animal having miRNA.

Also described herein are methods which include inducing certain cellular characteristics by providing to a cell a particular nucleic acid, such as a specific synthetic miRNA molecule or a synthetic miRNA inhibitor molecule. It is to be understood that the miRNA molecule or miRNA inhibitor need not be synthetic, but may have a sequence that is identical to a naturally occurring miRNA or they may not have any design modifications. In certain embodiments, the miRNA molecule and/or an miRNA inhibitor are synthetic, as discussed above.

It is also to be understood that the particular nucleic acid molecule provided to the cell is understood to correspond to a particular miRNA in the cell, and thus, the miRNA in the cell is referred to as the “corresponding miRNA.” In situations in which a named miRNA molecule is introduced into a cell, the corresponding miRNA will be understood to be the induced miRNA. It is contemplated, however, that the miRNA molecule provided introduced into a cell may not be a mature miRNA but can be capable of becoming a mature miRNA under the appropriate physiological conditions. In cases in which a particular corresponding miRNA is being inhibited by a miRNA inhibitor, the particular miRNA can be referred to as the targeted miRNA. It is contemplated that multiple corresponding miRNAs may be involved. In certain embodiments, more than one miRNA molecule can be introduced into a cell. Also, in certain embodiments, more than one miRNA inhibitor is introduced into a cell. In certain embodiments, a combination of miRNA molecule(s) and miRNA inhibitor(s) may be introduced into a cell.

Also described herein are methods which include identifying a cell or patient in need of inducing those cellular characteristics. It is to be understood that an amount of a synthetic nucleic acid that is provided to a cell or organism is an “effective amount,” which refers to an amount needed to achieve a desired goal, such as inducing a particular cellular characteristic(s). In certain embodiments of the methods include providing or introducing to a cell a nucleic acid molecule corresponding to a mature miRNA in the cell in an amount effective to achieve a desired physiological result.

Also described herein are methods which involve diagnosing a patient based on a miRNA expression profile. In certain embodiments, the elevation or reduction in the level of expression of a particular miRNA in a cell is correlated with a disease state compared to the expression level of that miRNA in a normal cell. This correlation allows for diagnostic methods to be carried out when that the expression level of a miRNA is measured in a biological sample being assessed and then compared to the expression level of a normal cell.

General Description

In IPF, proteins involved in abnormal wound repair leading to scarring of the lung are increased. There are no known genetic mutations to explain for these changes in protein expression. The inventors herein now show that a decrease in expression of regulatory microRNAs occurs to account for these alterations.

The microRNA cluster miR-17˜92 encodes seven microRNAs (miR-17-5p, miR-17-3p, miR-18, miR-19a, miR-19b, miR-20a, miR-92). The expression of each individual microRNA contained within the miR-17˜92 cluster from patients with IPF by quantitative RT-PCR as well as a mouse model was examined. Expression of miR-19b decreased in both mice and human pulmonary cells. Also, expression of miR-19b decreased proportionately with severity of disease in humans, thus showing that at least miR-19b is useful as a biomarker for IPF and as a therapeutic target and/or agent for IPF.

It is now shown herein that epigenetic silencing of miR-17˜92 occurred in lung tissue and fibroblast cell lines from patients with IPF due to enhanced DNA methylation. Diminished miR-17-92 expression inversely correlated to DNMT-1 expression. Introduction of the miR-17˜92 cluster in IPF lung fibroblasts reduced fibrotic gene and DNMT-1 expression, normalized cellular phenotype and reduced DNA methylation of the cluster.

It is also now shown herein that this regulation was conserved in mice. In a murine model of pulmonary fibrosis, enhancing miR-17˜92 expression using a demethylating agent in vivo reduced fibrotic gene and DNMT-1 expression suggesting augmented in vivo lung repair.

Also shown herein is the intimate interplay between miR-17˜92, DNMT-1 activity and lung fibrosis.

Also described herein are therapeutic approaches for the treatment of IPF.

In one embodiment, treatment of IPF lung fibroblasts with 5′-aza-2′-deoxycytidine (5′-aza) liberates miRNA expression and downregulates target mRNAs.

### EXAMPLES

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference. The following examples are intended to illustrate certain preferred embodiments of the invention and should not be interpreted to limit the scope of the invention as defined in the claims, unless so specified. In particular, the value of the present invention can thus be seen by reference to the Examples herein.

### Example 1

### Up- and Down-Related miR5

RNA isolated from human lung biopsies from patients with IPF were subjected to microRNA transcriptional profiling. From human IPF lung tissue, a significant decrease in expression of 23 known microRNAs was identified. A greater than 80% decrease in expression of miR-17, miR-19b and miR-20 encoded from the miR-17-92 cluster was detected.

A greater than a two-fold increase in the expression of 83 microRNAs, and five microRNAs were found to have a greater than 100-fold increase.

To directly examine the expression of the miR-17˜92 cluster, quantitative PCR was performed using specific primers to each of the microRNAs within the cluster. A 30-50% decrease in expression of miR-17, miR-19a, and miR-19b was found.

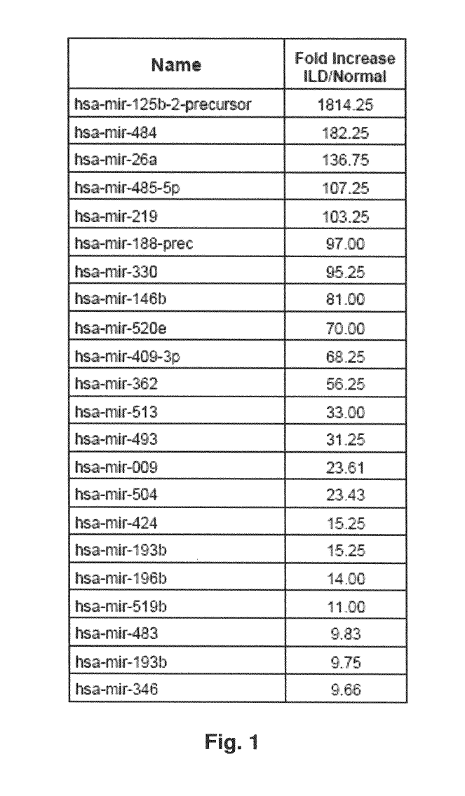

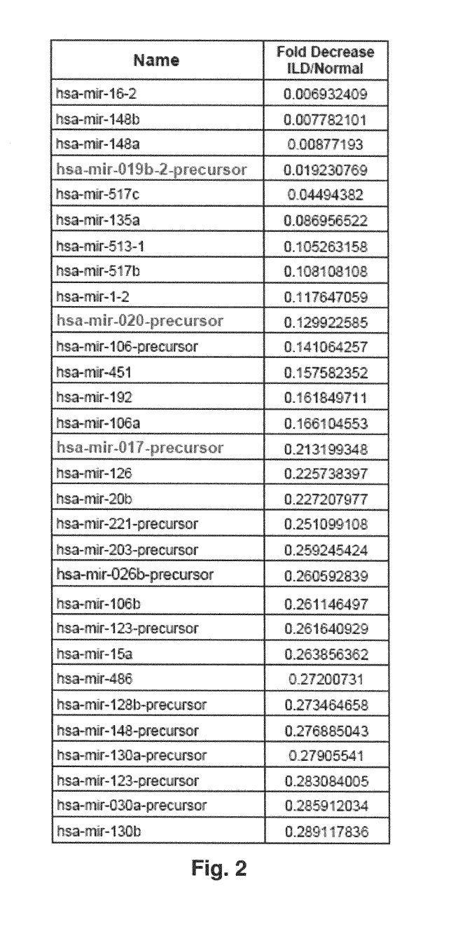

FIG. 1 and FIG. 2 show the total RNA from normal (NL) lung tissue or lung tissue from patients with interstitial lung disease (ILD)/IPF were subjected to miRNA transcriptional profiling. Relative expression of the microRNAs is shown as a ratio of ILD/normal. Highlighted miRNAs correspond to the miR-17˜92 cluster present on the chip. Shown is the average from two different donors per each group.

The expression of each of the miRNAs contained within the miR-17˜92 cluster in a mouse model of pulmonary fibrosis was analyzed. FIG. 1 and FIG. 2 show the decrease expression of miR-19b in a mouse model of pulmonary fibrosis. mRNA expression was examined from lung tissue from bleomycin-treated mice or vehicle (PBS) control treated mice by quantitative RT-PCR. Relative expression was normalized to 18s RNA control. Shown is the average±S.E.M, from eight mice per group.

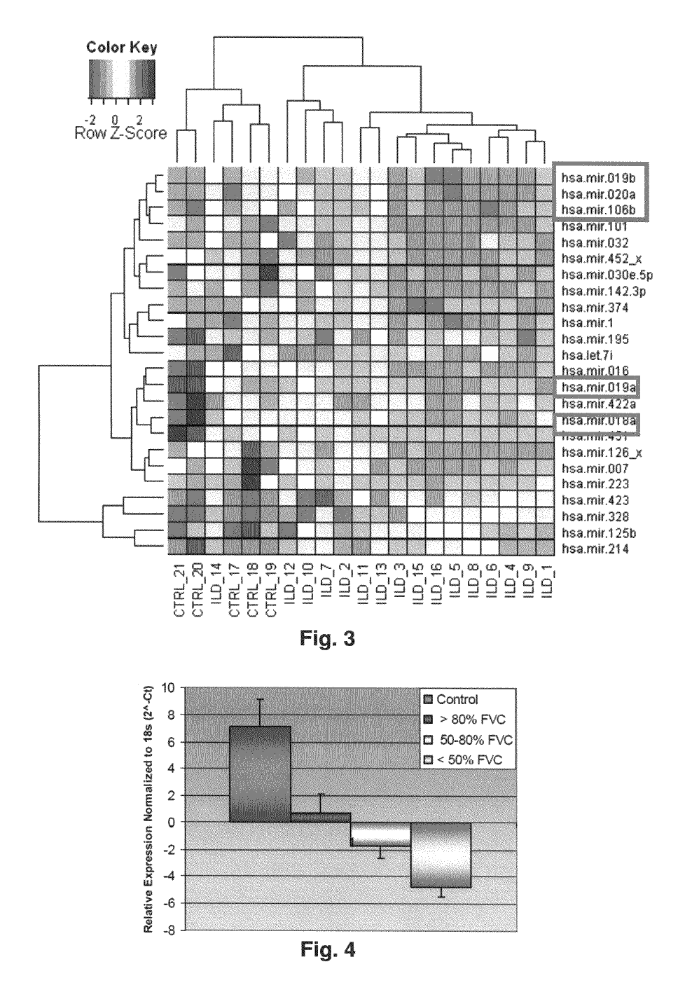

MicroRNA expression profiles from human patients with interstitial lung disease (ILD)/IPF and control (CTRL) lung tissue were analyzed. The ILD/IPF lung tissues were divided into three categories according to severity of disease based on forced vital capacity (FVC): group 1—<50% (most severe); group 2—50-80%; and, group 3—>80%. The unsupervised hierarchical clustering results for 16 ILD/IPF patient samples and 5 control samples are shown in FIG. 3. The majority of the control and IPF samples had similar expression profiles as indicated with the samples clustering together. Notably, a decrease in expression of miR-19b and miR-20a in the ILD/IPF samples was detected, as compared to the control tissue. FIG. 4 shows the hierarchical cluster analysis of miRNAs in lung tissue from patients with ILD and normal tissue (CTRL). Correction of the raw PCR cycle threshold (CT) scores included geometric mean normalization. There were 24 miRNAs that were differentially expressed among the samples as determined by Student's t-test followed by the Benjamini & Hochberg multiple test correction. Color key: dark green, highest expression; dark red, lowest expression.

Since a similar decrease of miR-19b in both mouse and human pulmonary fibrosis samples was observed, this decrease was further validated. Validation of decrease targets involved using increase RNA for the quantitative RT-PCR. A consistent decrease in miR-19b is shown with increasing severity of IPF, and is now shown herein to be a marker of disease progression.

FIG. 4 shows the validation of expression of miR-19b in ILD tissue. To confirm the observed decrease in miR-19b expression from profiling data, total mRNA was increased in the real-time PCR reaction by 100-fold. The reaction was repeated using a different Applied Biosystems 7900HT real-time PCR instrument and a 96 well format. Shown is the average relative expression normalized to 18s internal control ±S.E.M (Control n=6, >80% FVC n=8, 50-80% FVC n=6, and <50% FVC n=3). These data show that the loss of miR-19b expression is useful to predict disease progression.

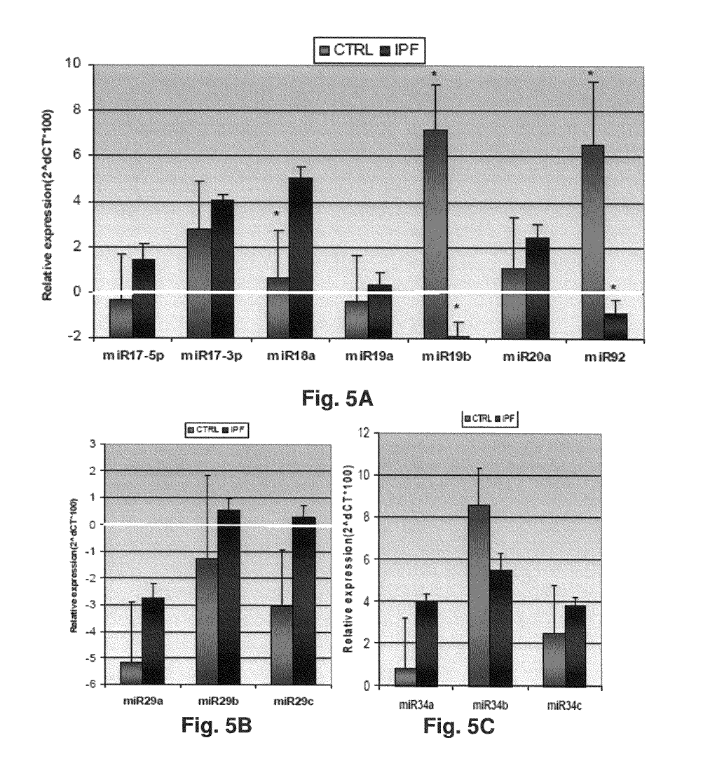

FIG. 5A-FIG. 5C show the validation of microRNA expression in human IPF by quantitative (q)RT-PCR. RNA from control (CTRL) or interstitial lung disease (ILD)/IPF lung tissue was subjected to qRT-PCR using specific primers of each microRNA. Relative expression was determined using 18s RNA as an internal control. FIG. 5A shows the expression of the miR-17˜92 cluster in IPF. FIG. 5B shows the increase the expression of miR-29 family in IPF samples compared to control samples. FIG. 5C shows the expression of miR-34 family in IPF samples. Data shown are the average±SEM (CTRL n=6, IPF n=17). P<0.05.

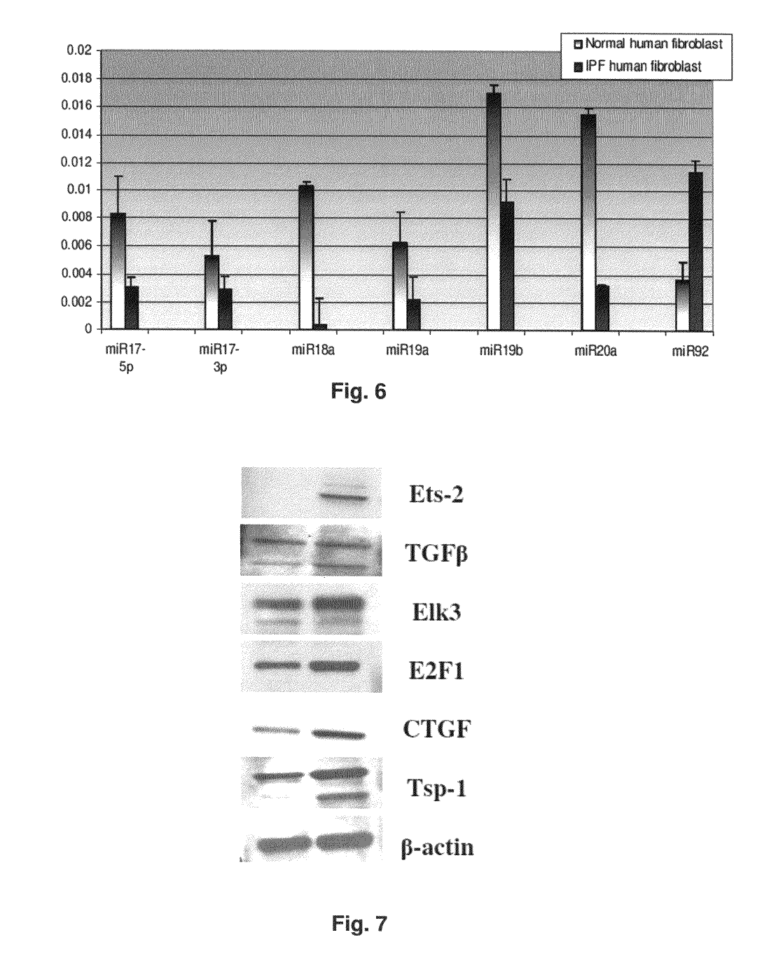

FIG. 6 is a graph showing miR-17˜92 expression in human lung fibroblast for normal and IPF.

FIG. 7 shows a comparison between normal lung fibroblast and IPF lung fibroblast for Ets-2, TGFβ, Elk3, E2F1, CTGF, Tsp-1 and β-action.

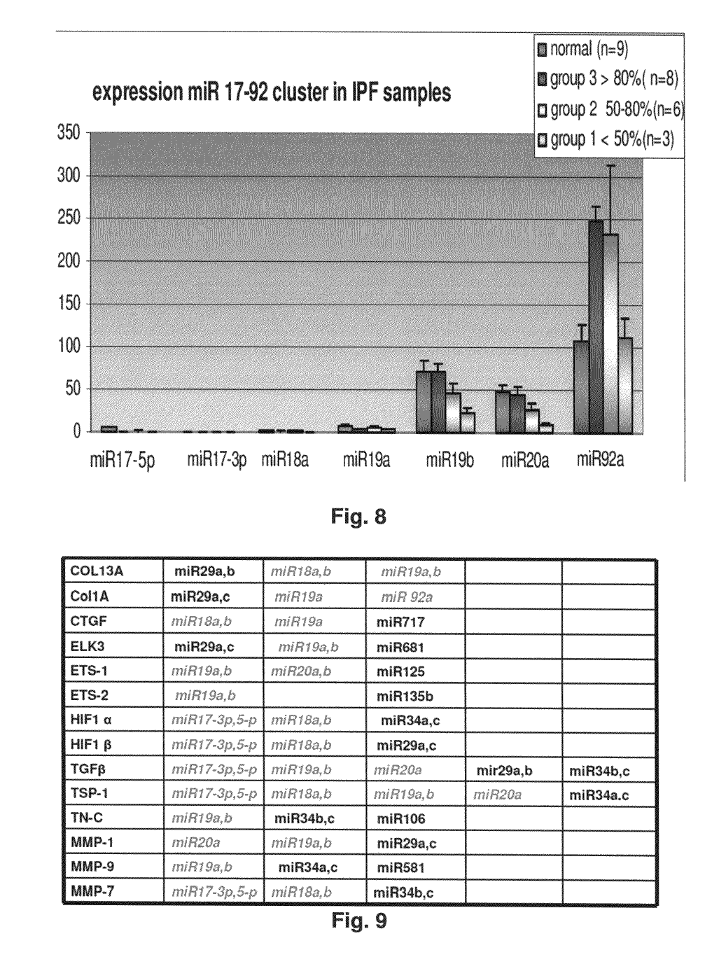

FIG. 8 is a graph showing miR-17˜92 cluster in IPF samples, and in particular, the miR-19b expression and miR-20a expression. Also, expression of miR-34b was decreased but the other miR-34 family members or miR-29 cluster are not; therefore, these microRNAs do not play a major role IPF (data not shown).

### Example 2

### mRNA Profiling of IPF Patients

Lung tissue was obtained from the Lung Tissue Research Consortium and these patients were stratified by a number of different quantitative metrics, including lung function testing. Distinct mRNA expression profiles distinguishing patients with IPF/ILD from controls (normals and COPD samples) were found. FIG. 9 shows the microRNAs which regulate gene expression involved in IPF.

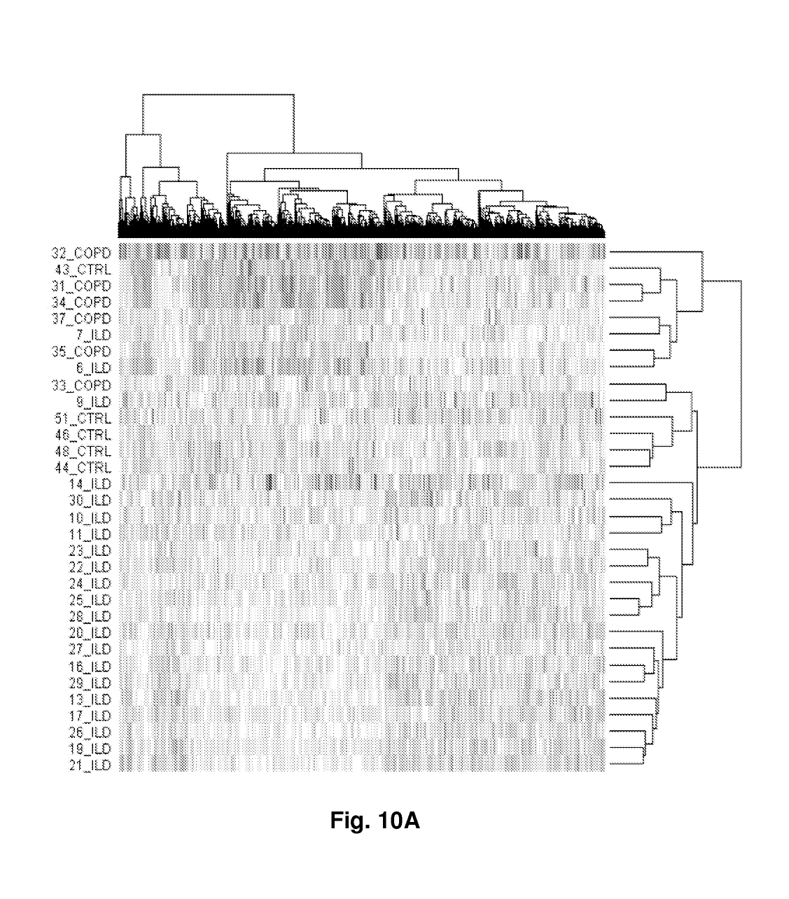

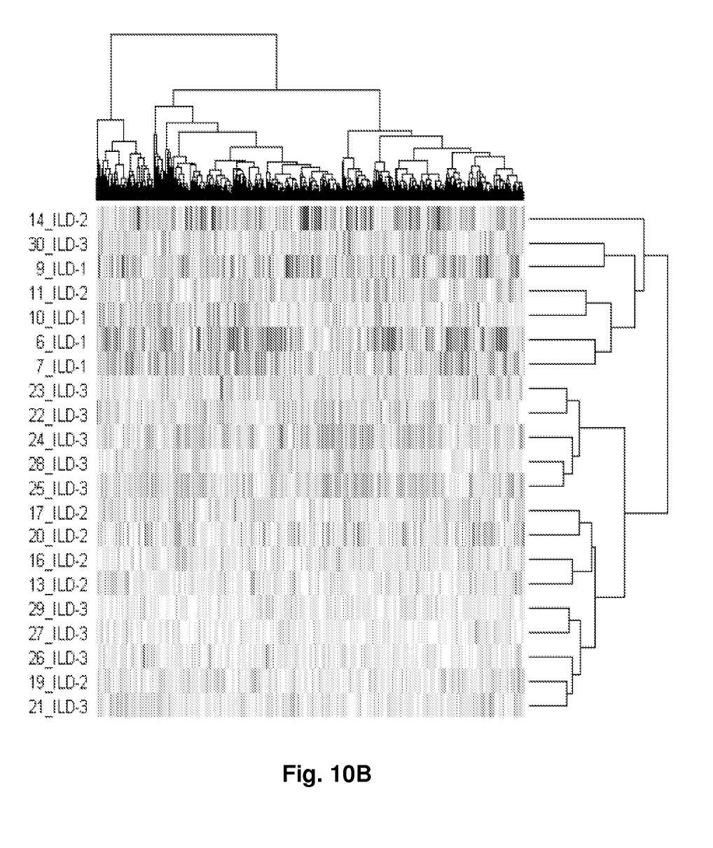

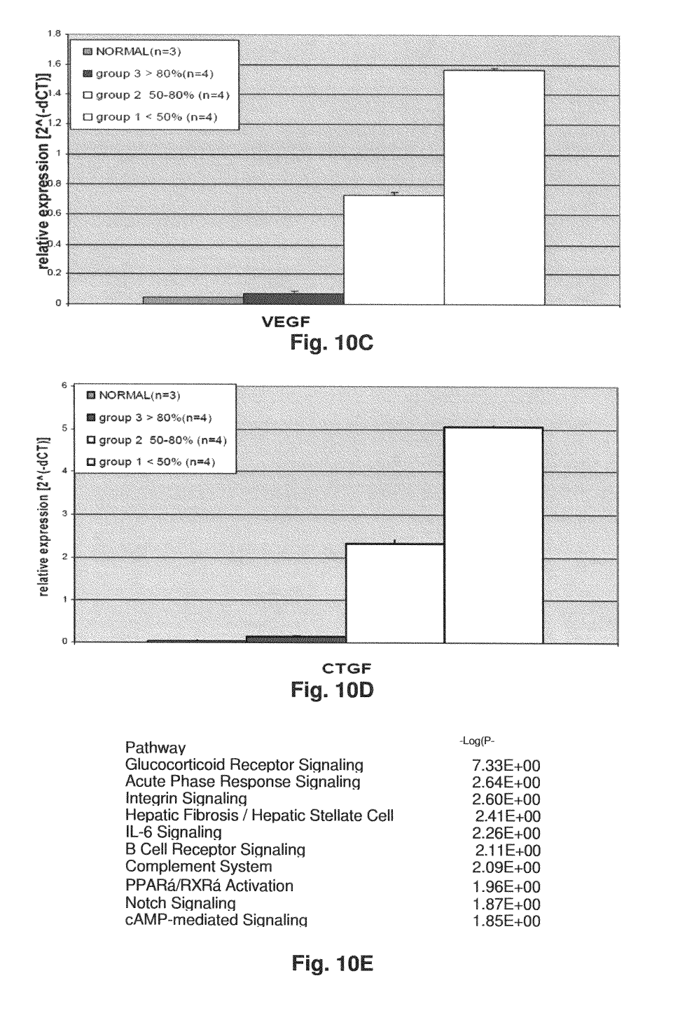

As shown in FIG. 10A, the unsupervised cluster analysis resulted in 18 of the 21 profiles from IPF/ILD patients grouped together (on the right side of the figure), while 4 of the 7 COPD profiles were grouped together on the left of the figure. Quantitative phenotyping data were used to stratify the data, including stratification by the forced vital capacity (FVC pre-bronchodilator, % predicted). FIG. 10B shows mRNA clustering of IPF/NSIP patients compared to normals and COPD lung tissue mRNA profiles. Quality assurance checks included examination of RNA integrity, cDNA yield after amplification, visual inspection of the Affymetrix raw data files (for a high background or other hybridization artifact), and study of the final profiles for outliers.

Several genes elevated in patients with IPF include CTGF and VEGF and the expression of these genes in the patient samples was examined. As shown in FIG. 10C-FIG. 10D, both VEGF and CTGF increased in expression with worse disease. The highest expression level was observed in the most severe cases (<50% FVC).

Further, while protein levels for c-myc and CTGF were increased, HDAC4 was decreased in the mouse model of pulmonary fibrosis. Also, HDAC4 expression can be regulated by miR-17-5p, miR-20a, and miR-19a, all of which are increased in both human and mouse pulmonary fibrosis. Also, that miR-19b is useful as a prognostic indicator of an IPF disease state, as well as at a target for therapy for IPF.

### Example 3

### Bleomycin Treatment

While an increase in the expression of several of the miRNAs (miR-19a and miR-20a) was observed, a significant decrease in the expression of miR-19b in mice treated with bleomycin compared to control mice was found. Also, an increase in CTGF protein expression in the lung from bleomycin-treated mice was found.

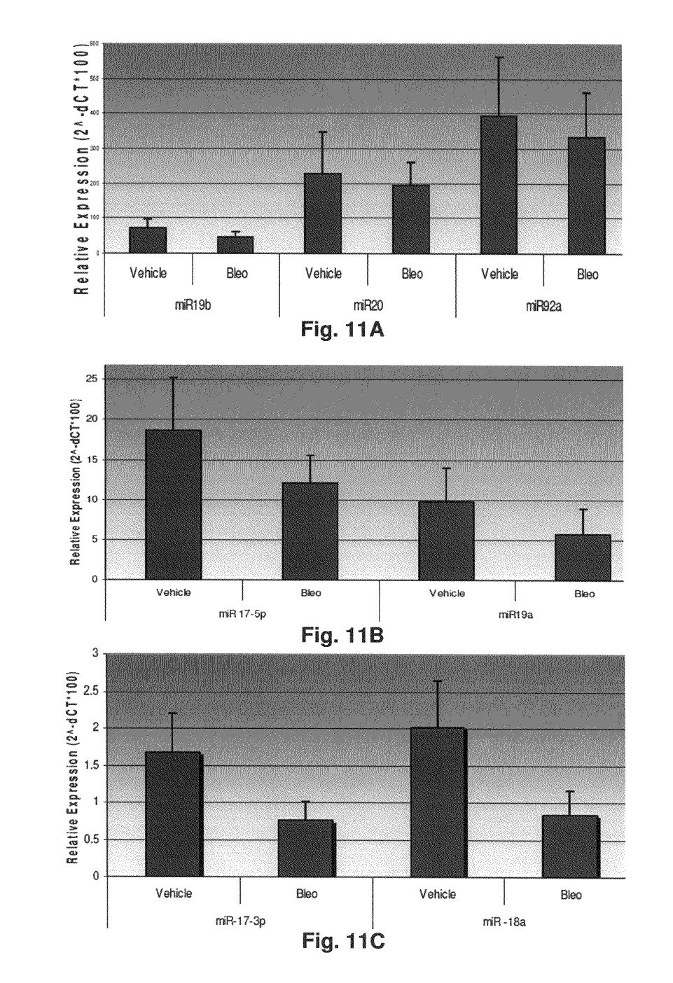

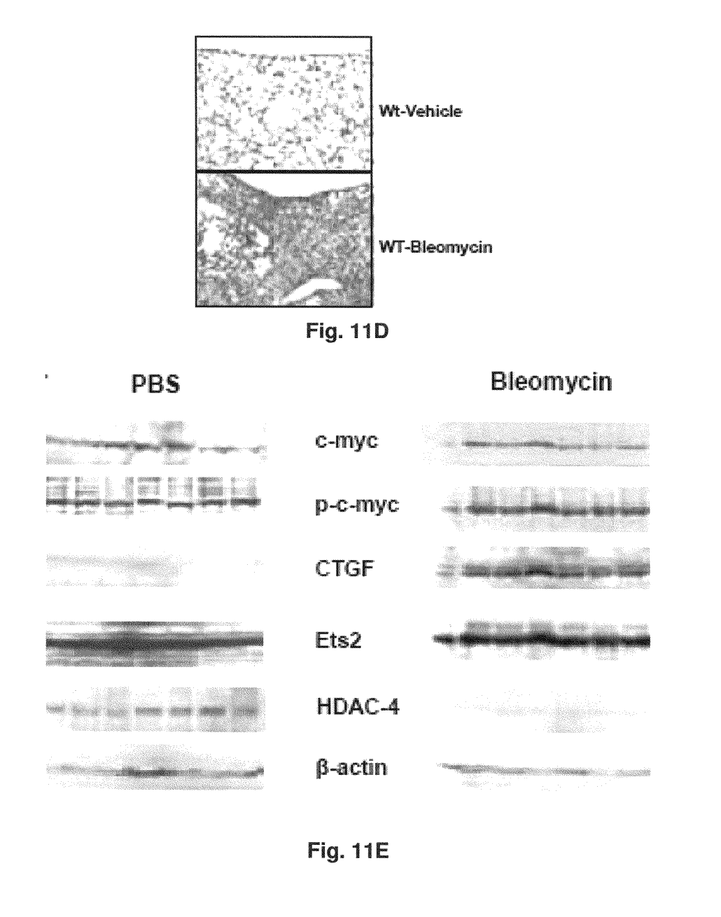

FIG. 11A-FIG. 11C are graphs showing the decreased expression of the miR-17˜92 cluster in lung tissue from FVBM mice treated with bleomycin.

The pathological and protein assessment of bleomycin-induced fibrosis in mice. FIG. 11D shows the trichrome staining confirmed collagen deposition in the lungs of mice treated with bleomycin. Shown is a representative image. FIG. 11E shows that a Western blot analysis was performed to examine CTGF, c-myc, phosphorylated c-myc Ets2 and HDAC4. Shown are representative data from seven mice per treatment group.

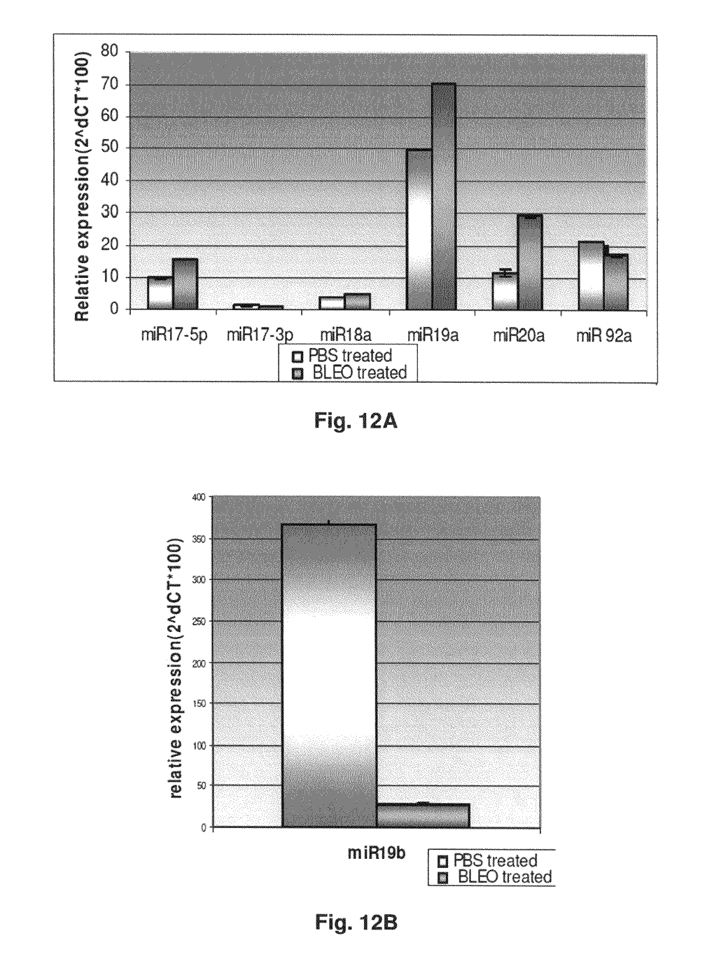

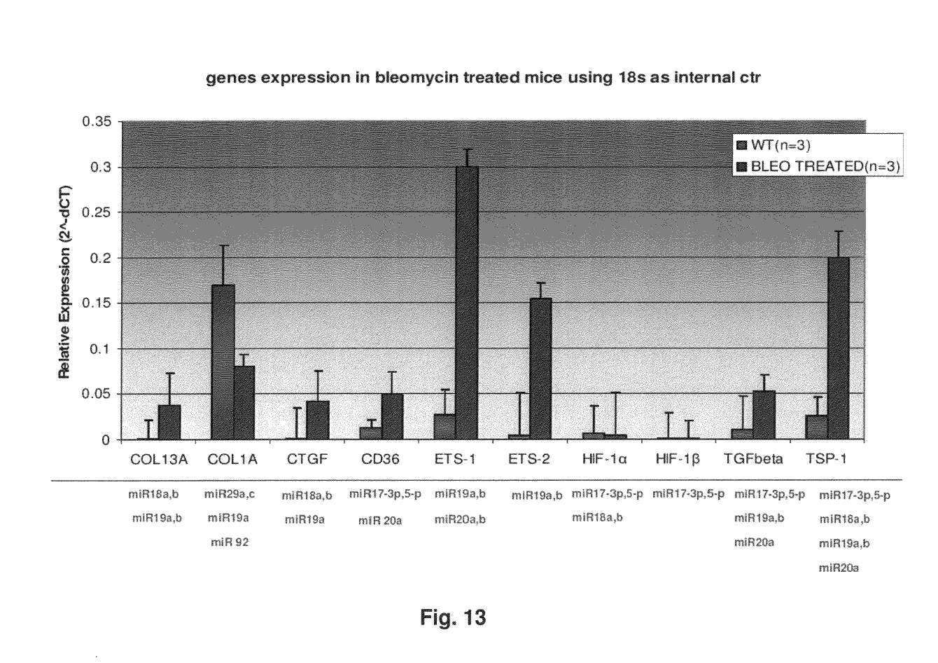

FIGS. 12A-FIG. 12B are graphs showing changes in expression of the miR-17˜92 cluster in bleomycin-induced fibrosis in C57BL/6 mice. RNA isolated from the lungs of mice was subjected to qRT-PCR using specific primers to each microRNA in the cluster. Relative expression was determined using 18s RNA as an internal control. Data shown are the average ±SEM (n=8). FIG. 13 is a graph showing IPF gene expression in bleomycin treated C67BL/6 mice using 18s as an internal control.

### Example 4

### Gene Expression in Human Lung Fibroblasts

Genes facilitating myofibroblast proliferation, extracellular matrix synthesis, developmental pathways, and angiogenesic gene expression were identified. Genes implicated in these pathways strongly support mesenchymal cell activation and proliferation, but do not allow discrimination among the proposed origins of the regulation of this (myo)fibroblast activity; recruitment of fibroblasts/fibrocytes from the circulation, or the presence of “Epithelial cell to Mesenchymal cell Transition” (EMT). Other active genes such as VEGF and Notch signaling are consistent with active or aberrant developmental programs, angiogenenic programs and endothelial cell targeting and turnover. The genes responsible for triggering the “hepatic fibrosis/stellate cell activation’ pathway emphasize the importance of TGF-β, TGF-α, EGF, and endothelin signaling in the IPF/ILD samples. These signaling molecules in turn regulate many of the effectors of extracellular matrix remodeling including type I and type III collagen, and matrix metalloproteinase-2 and matrix metalloproteinase-7.

The samples profiled for mRNA were also profiled for miRNA by a RT-PCR based method. This analysis demonstrates the ability to capture miRNA profiles from frozen samples, stratify the data, and relate the miRNA profiles to mRNA profiles. Hierarchical analysis of the IPF/ILD data by FVC functional group suggests emergence of specific miRNA profiles.

miR-19b, miR-20a, and miR-106b are highly expressed in control lung tissue, but are markedly reduced in lung tissue from patients with IPF/ILD. These miRNA profiles implicate the miR-17˜92 cluster as a novel target that is reduced in patients with IPF/ILD. Reduced expression of this miRNA cluster may be used to enhance expression of gene networks targeted by these miRNAs.

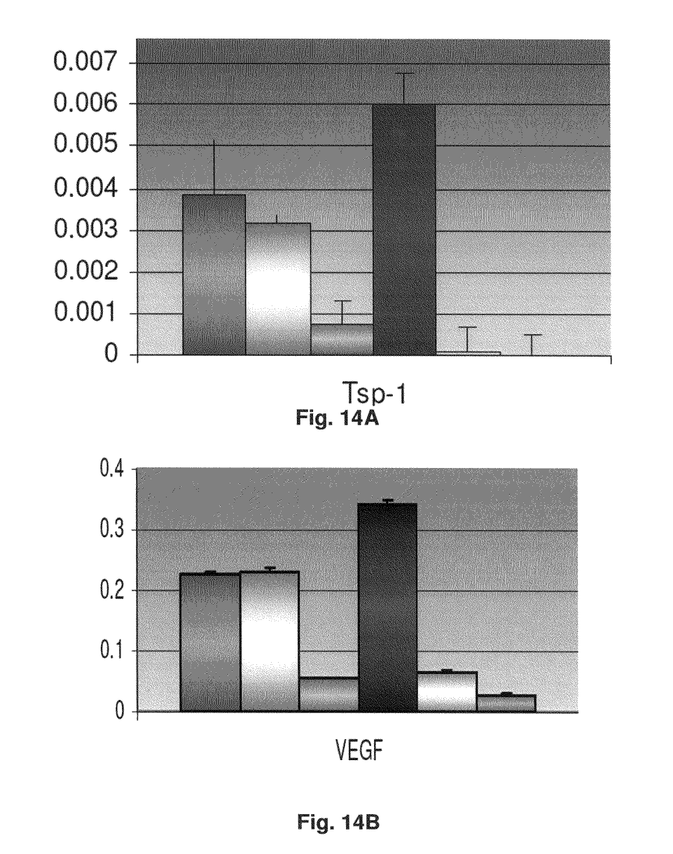

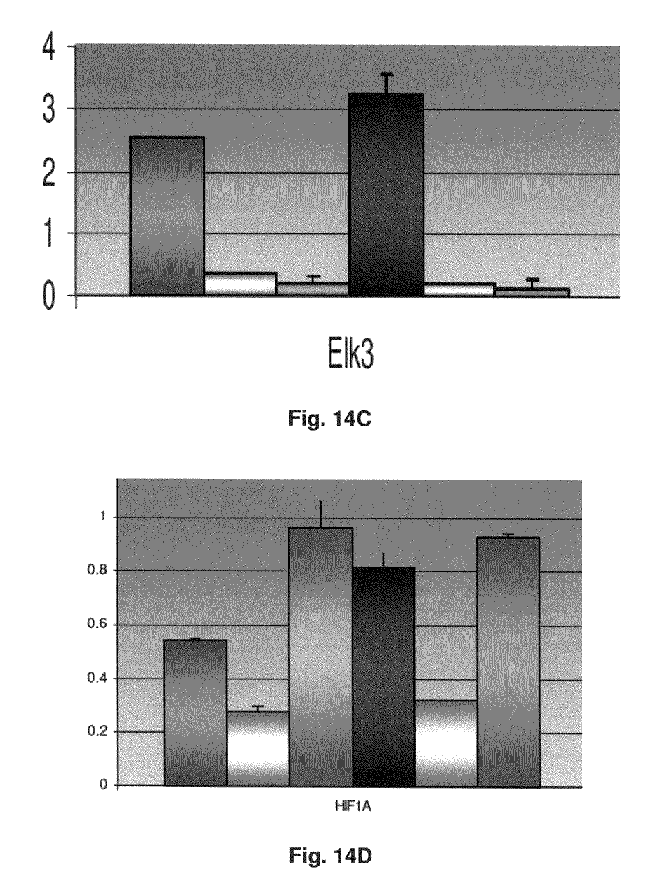

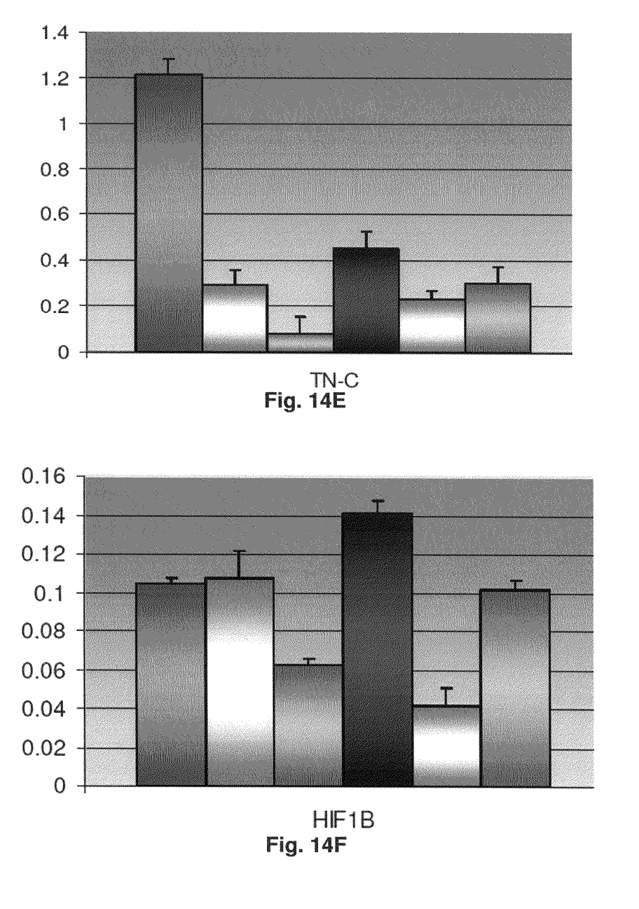

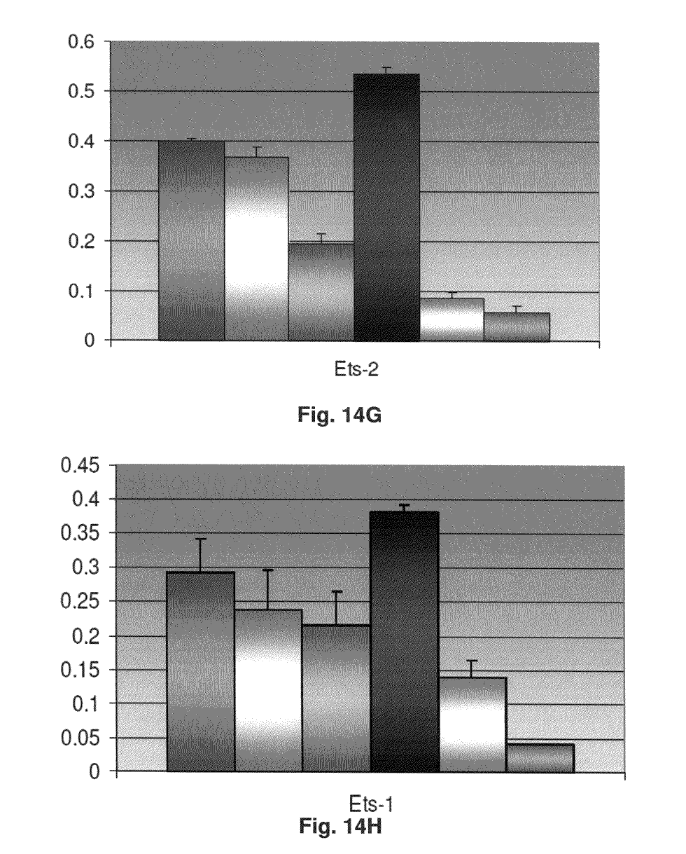

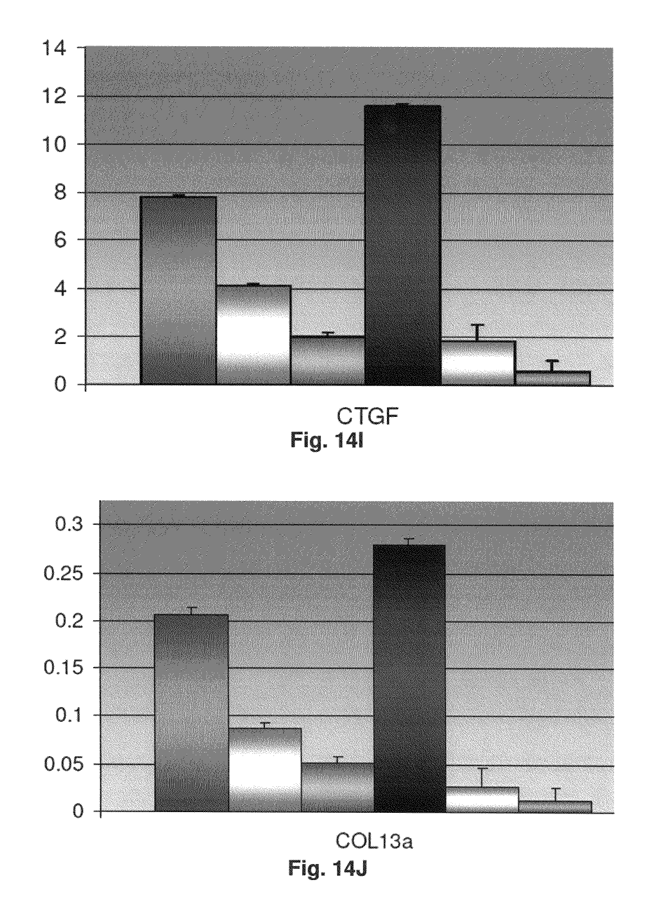

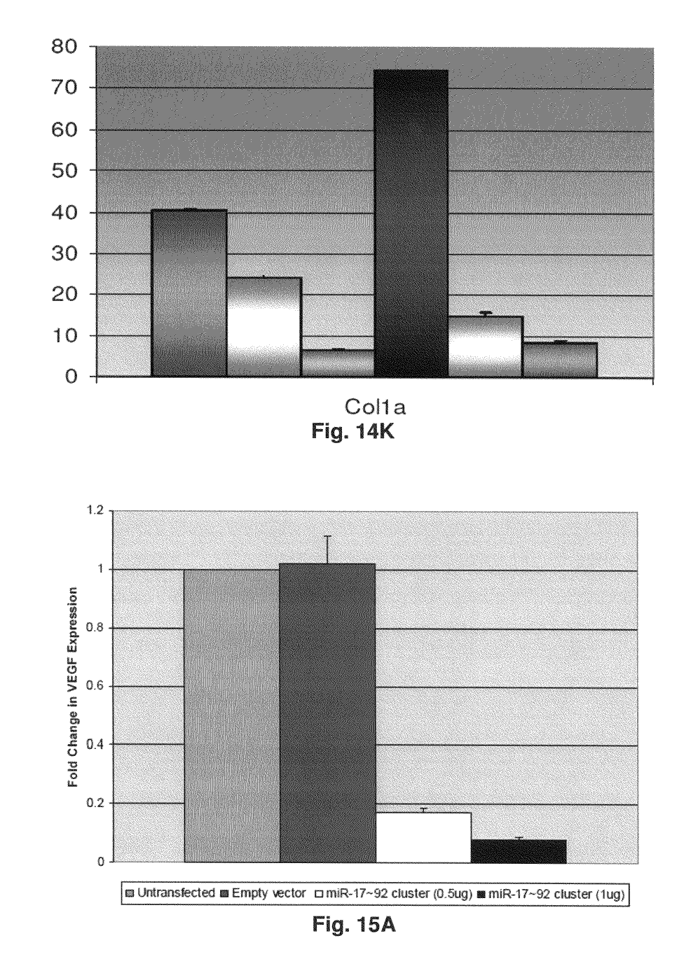

FIGS. 14A-14K are graphs showing the effect of over-expression of miR-17˜92 cluster on IPF gene expression for Tsp-1, VEGF, Elk3, HIF1A, TN-C, HIF1B, Ets-2, Ets-1, CTGF, COL13a and Col1a. The mean gene expression from the IPF/ILD profiles was calculated, and these values were divided by the mean expression observed in the control samples. These values were used to identify key biological pathways that are likely to be active in the IPF/ILD patients. Ingenuity software analysis scored ten pathways with acceptable P-values of 10⁻².

### Example 5

### Re-Introduction of miR5

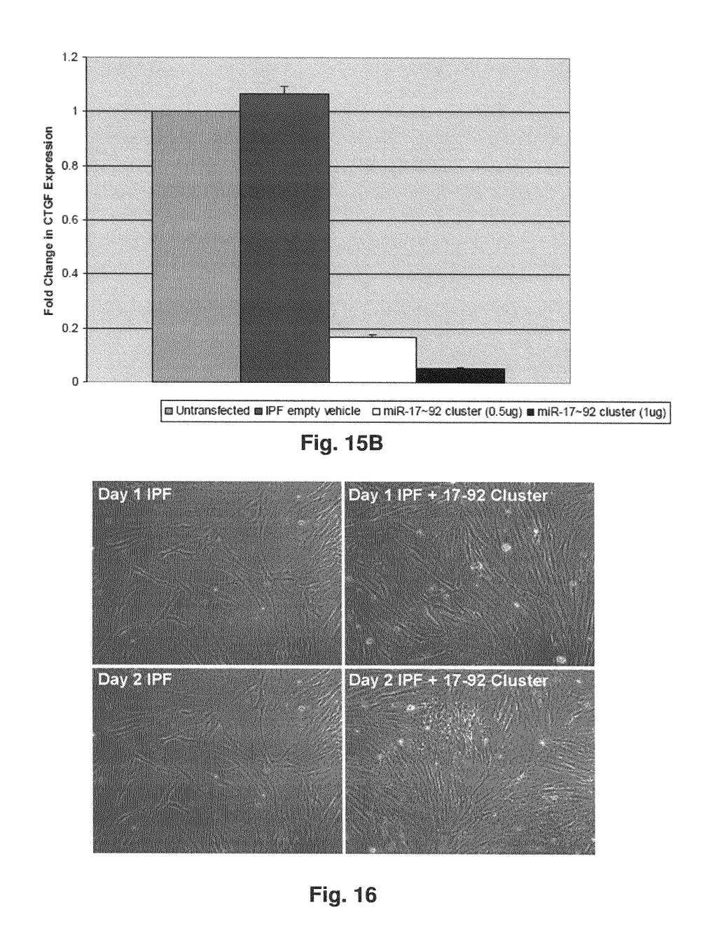

Re-introduction of the miR-17˜92 cluster in the IPF cell line decreases the expression of these gene targets. The initial enhanced expression of VEGF and CTGF were markedly reduced by re-introduction of the miR-17˜92 cluster in fibroblasts derived from IPF patients lungs (FIG. 15A-FIG. 15B). This demonstrates that these findings are not patient or cell line specific. Also, distinct phenotypic changes in cells transfected with the cluster compared untransfected cells were found, as shown in FIG. 16, which shows that the fibroblasts appeared to organize in a contiguous cell sheet.

By using two methods of detection the microarray chip and qRT-PCR, many differences in the miRNA expression were found. miR-19b was consistently decreased between the two methods. A high similarity in expression of miR-17˜92 cluster was found between human and mouse. Increases in CTGF protein in IPF are now shown herein to be due to decreases in expression of the miR-19b from the miR-17˜92 cluster. In addition, miR-19b and miR-20a are down regulated with increasing severity of disease in patients with IPF.

### Example 6

### Targeted Genes

Several genes were identified that are targeted by the miR-17˜92 cluster. The expression of these genes as well, as corresponding protein lung tissue based on disease severity, were examined

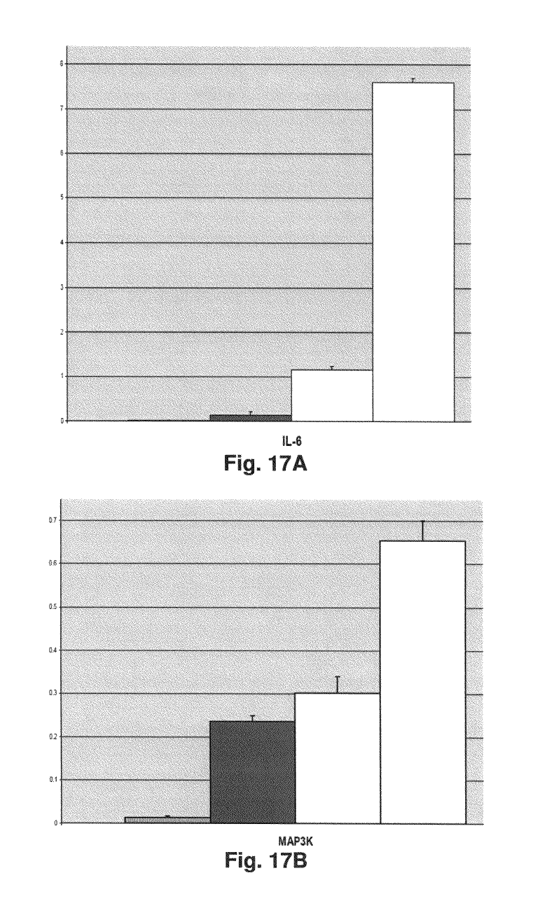

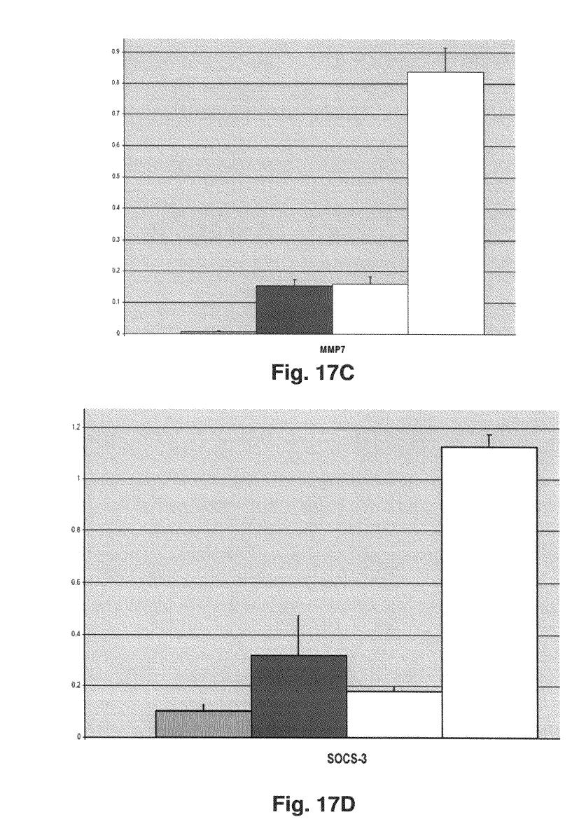

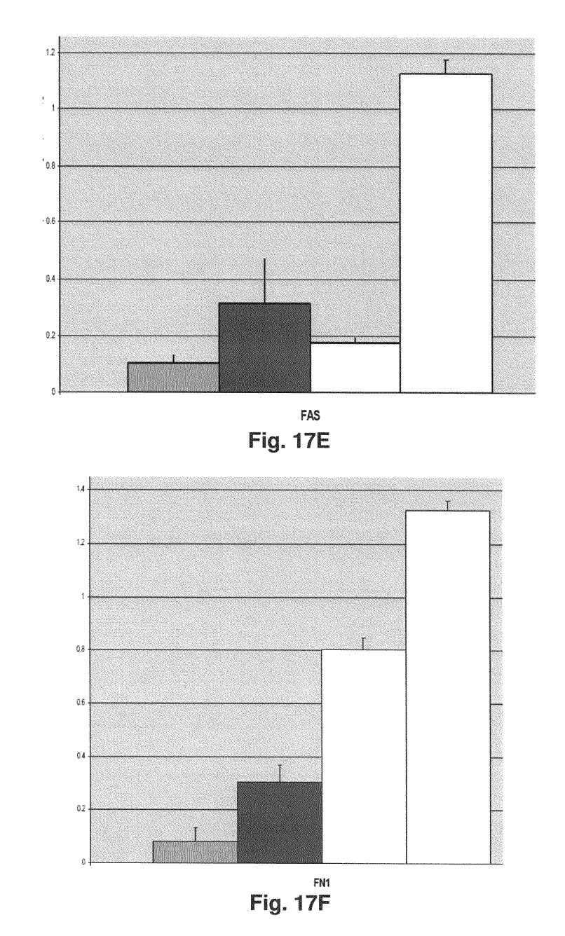

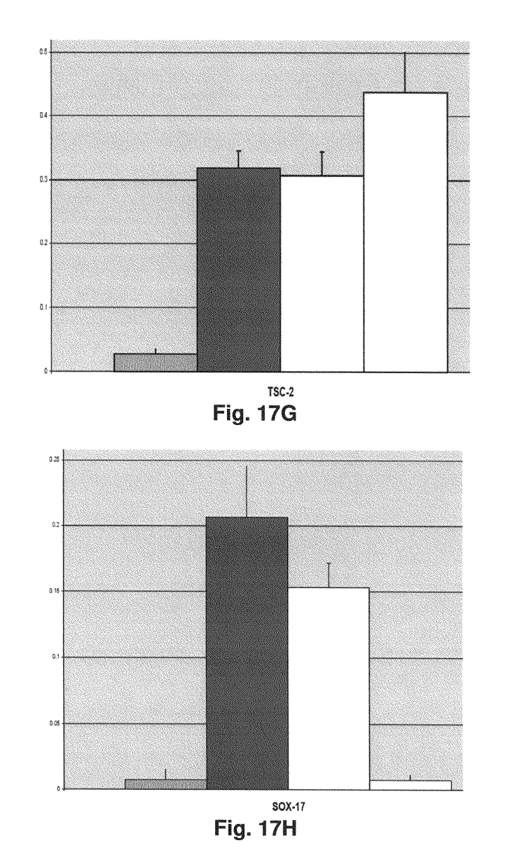

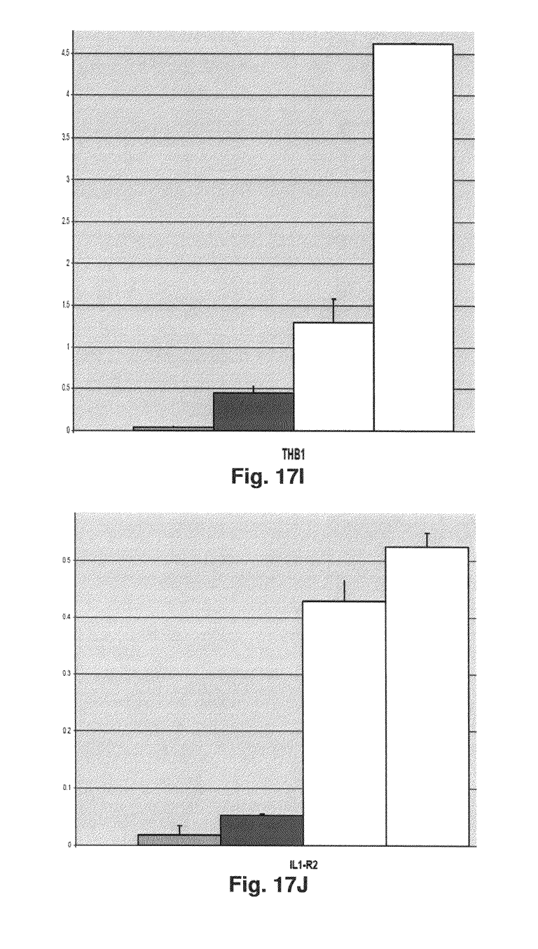

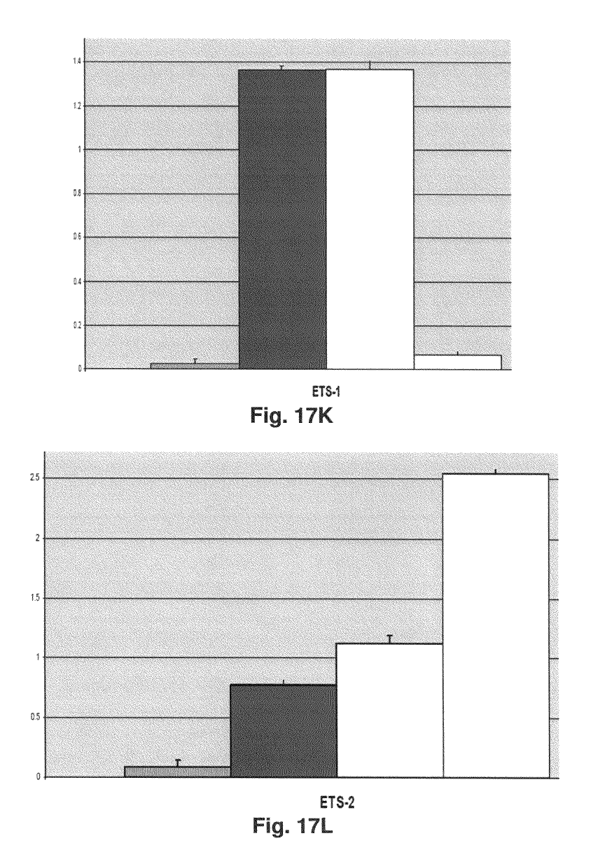

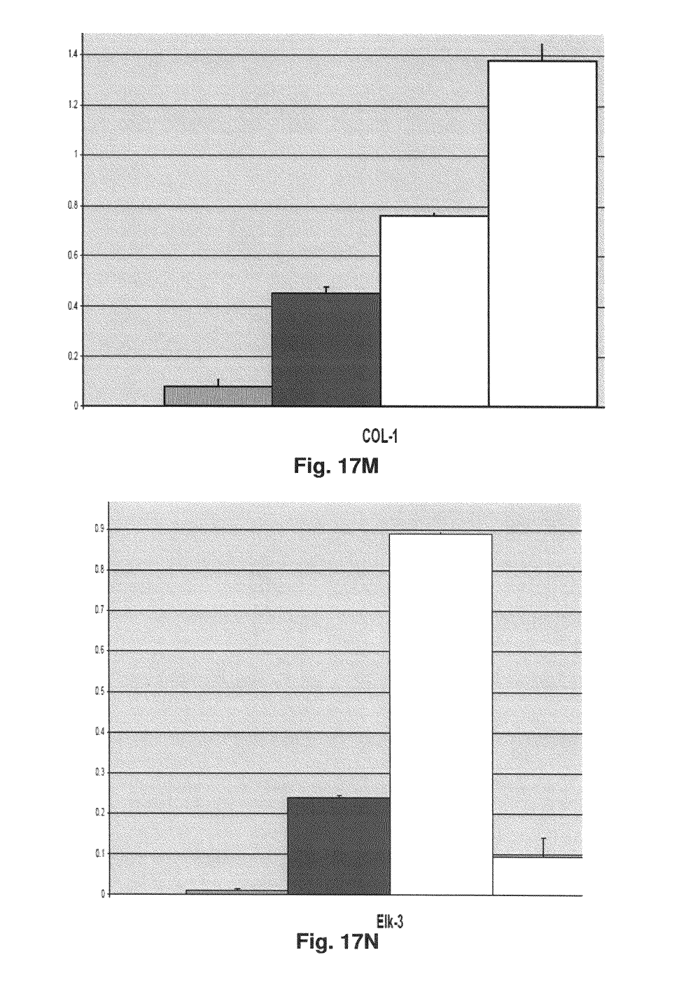

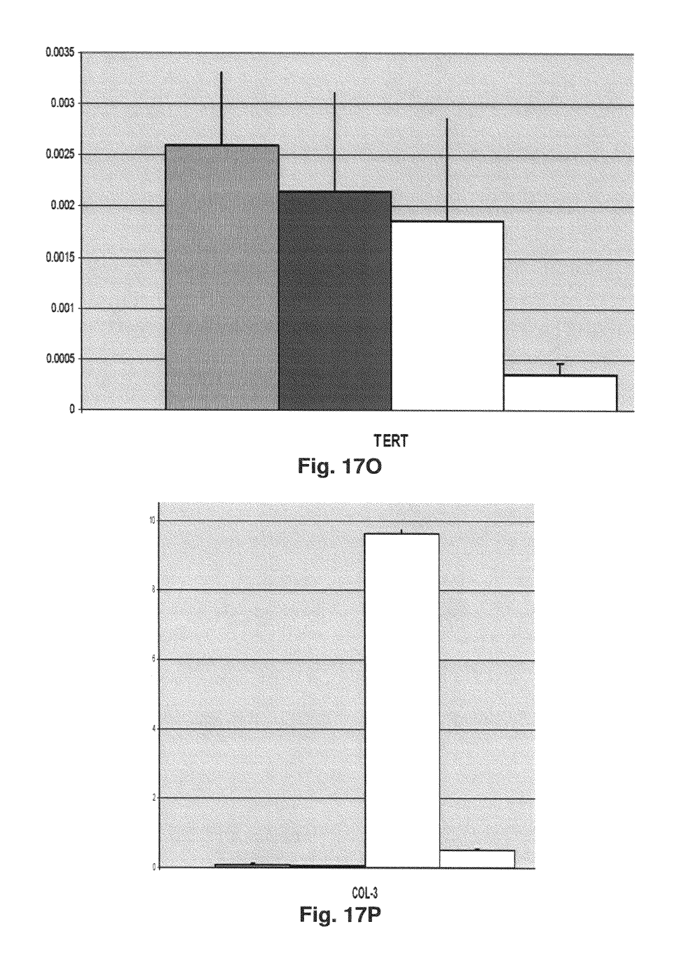

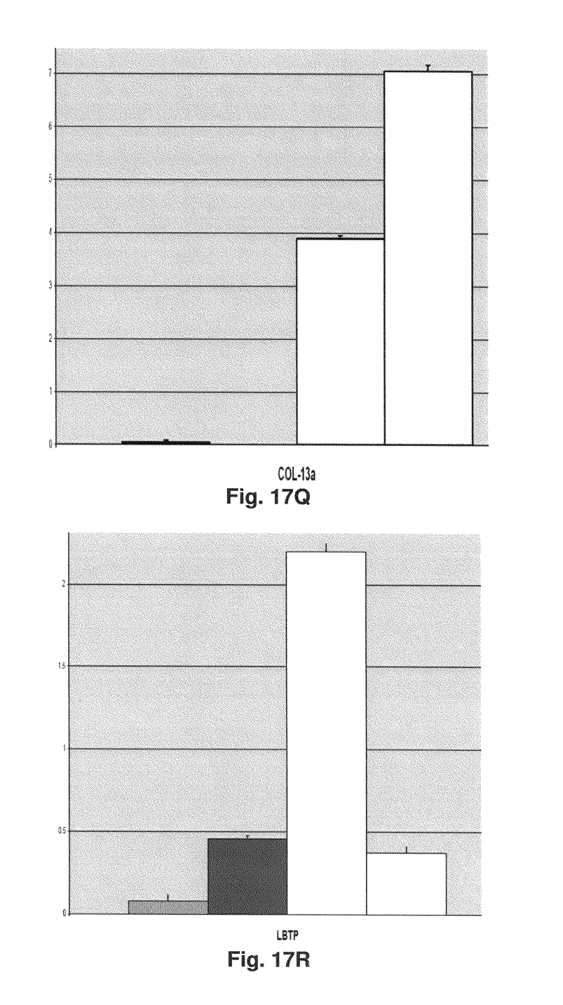

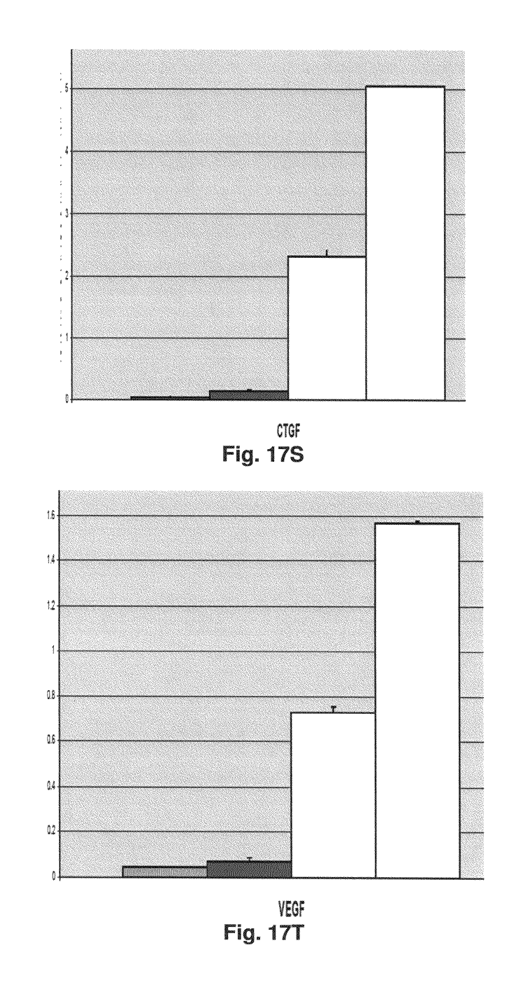

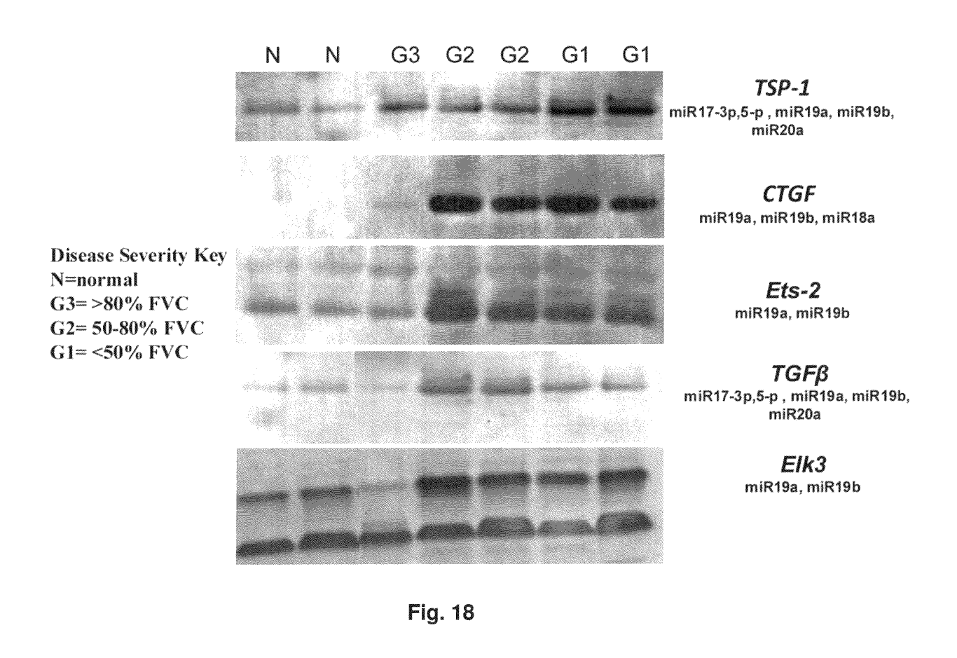

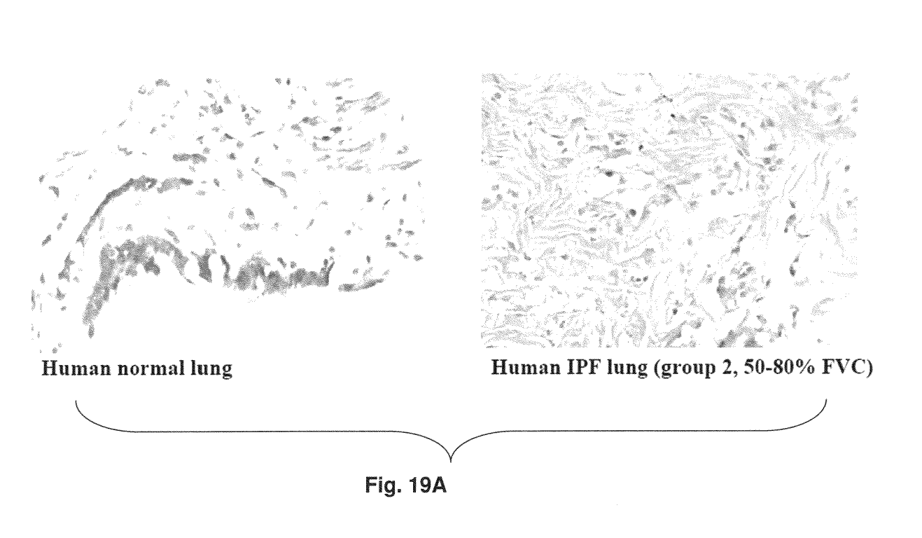

FIGS. 17A-17T show graphs for gene expression in patient samples based on disease severity. FIG. 18 shows protein expression in lung tissue from patients with IPF.

In situ Hybridization

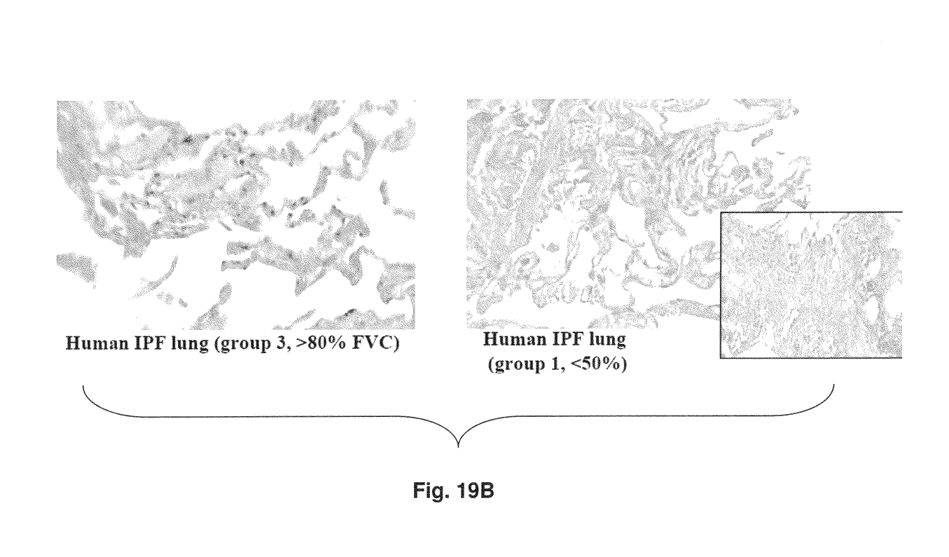

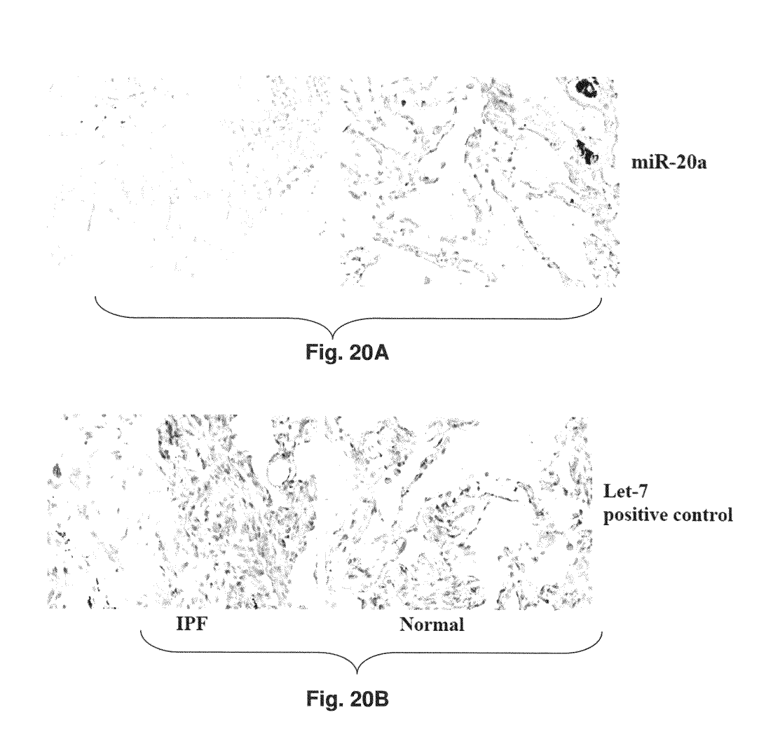

In situ hybridization was conducted to confirm qRT-PCR analysis that expression of miR-19b and miR-20a are decreased in lung tissue from patients with IPF compared to normal tissue. For example, FIGS. 19A-19B show the miR-19b expression in human lung tissue, and FIGS. 20A-20B show the miR-20a and Let-7 expression in human lung tissue.

Expression in Cell Lines

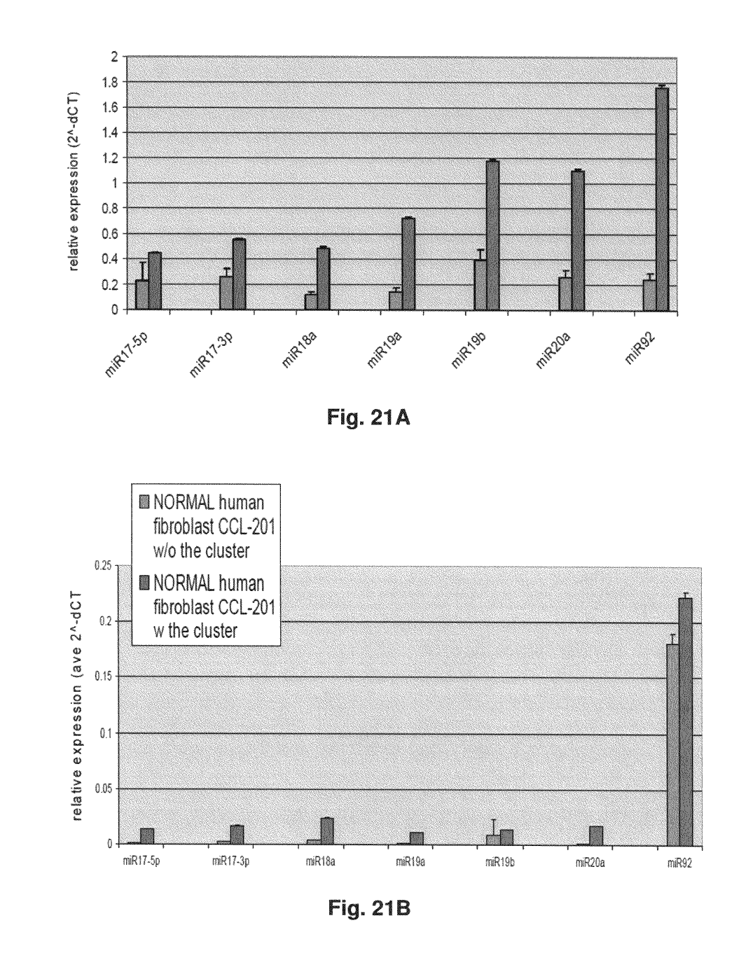

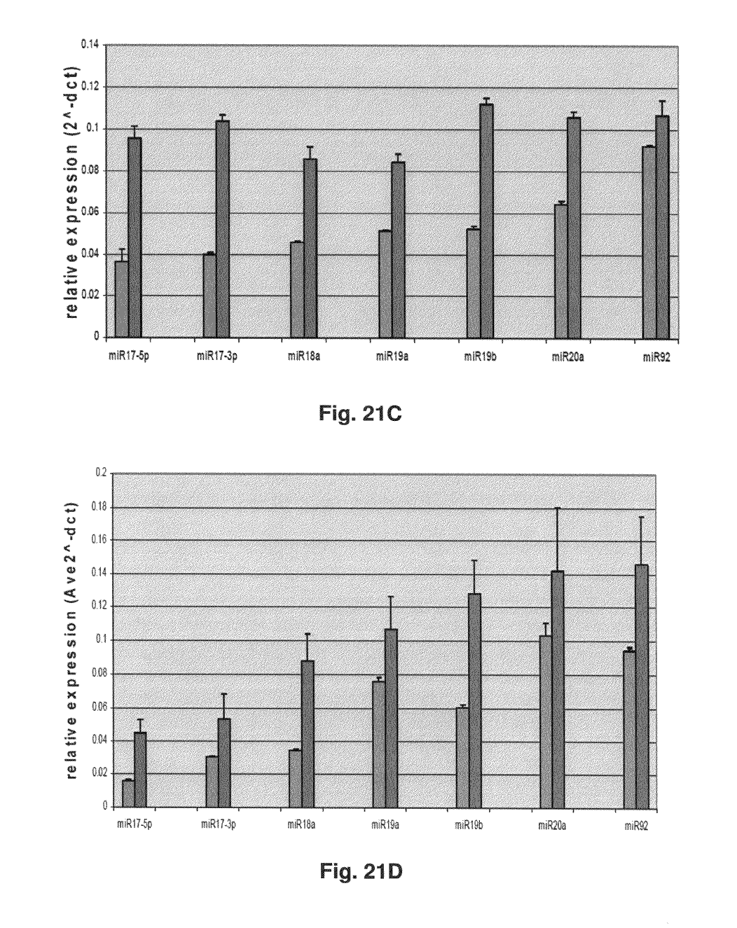

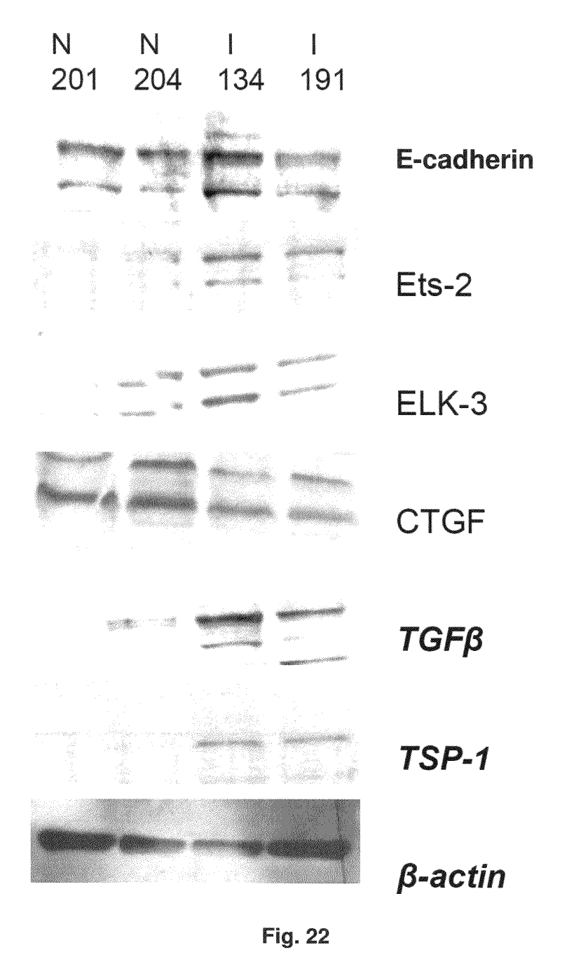

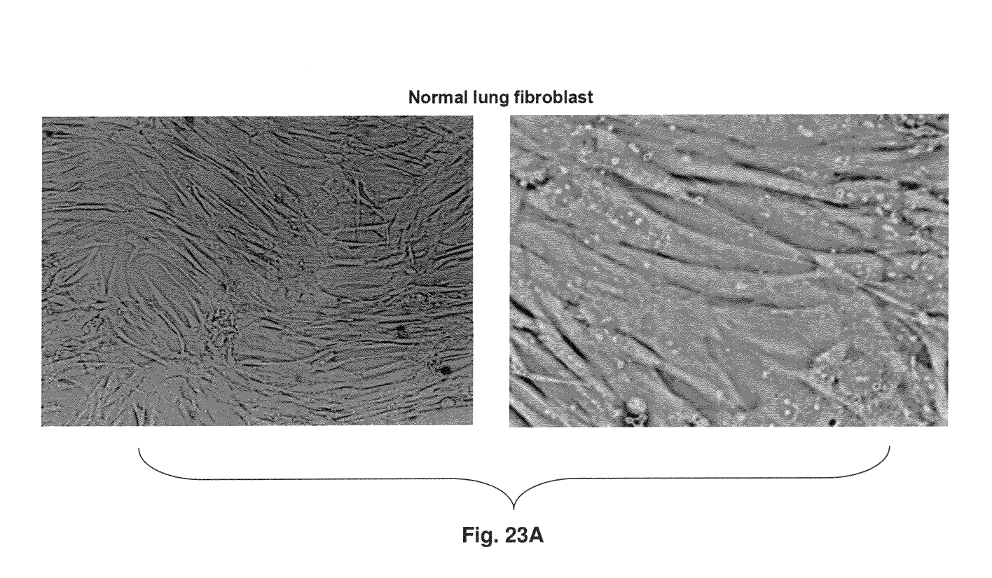

Decrease expression of the miR-17˜92 cluster in lung fibroblast cell lines derived from patients IPF compared to normal human lung fibroblast cell lines. FIGS. 21A-21D are graphs showing the miRNA expression in human lung fibroblast cell lines. Protein expression in human normal and IPF lung fibroblast cell lines is shown in FIG. 22, where N=Normal, and I=IPF.

### Example 7

### Phenotypic Differences

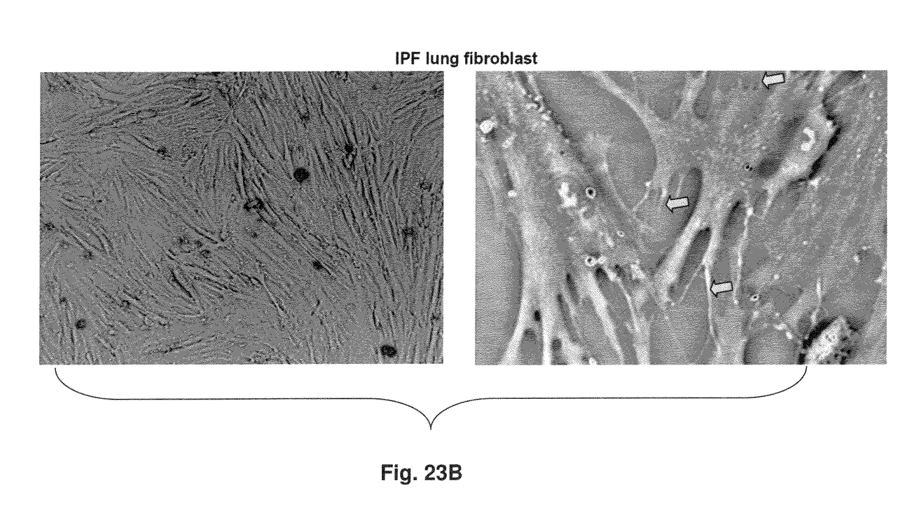

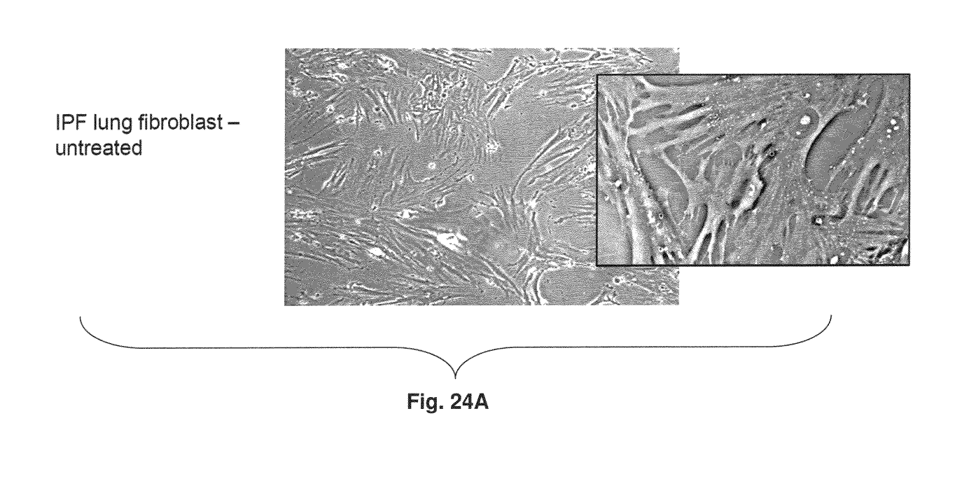

IPF-derived lung fibroblasts appear phenotypically different with more filipodia compared to normal human lung fibroblast cell lines. FIGS. 23A-23B show the morphology for human lung fibroblasts in Normal and IPF.

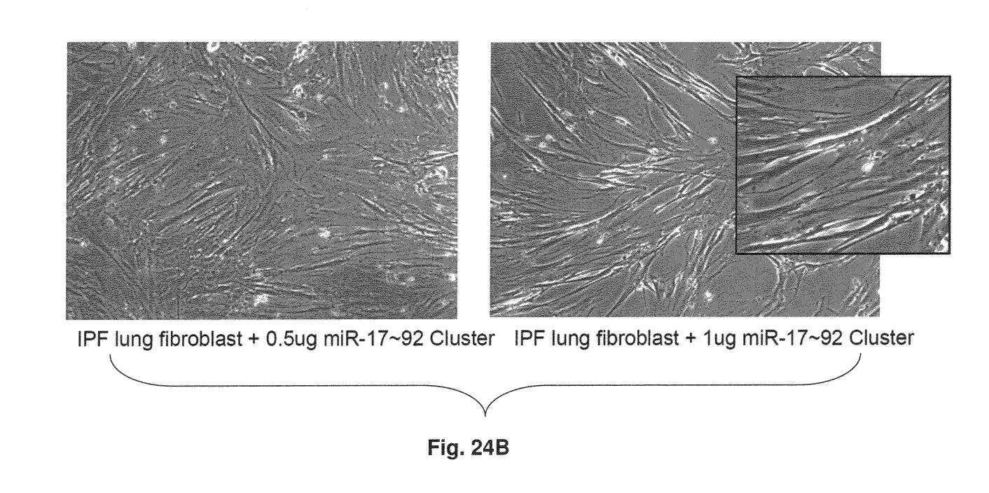

The miR-17˜92 cluster, as well as miR-19b and miR-20a, are decreased in the IPF-derived fibroblasts similar to tissue from patients with IPF. FIGS. 24A-24B show IPF-derived fibroblasts transfected with the miR-17˜92 cluster begin to assume a phenotype similar to normal lung fibroblasts.

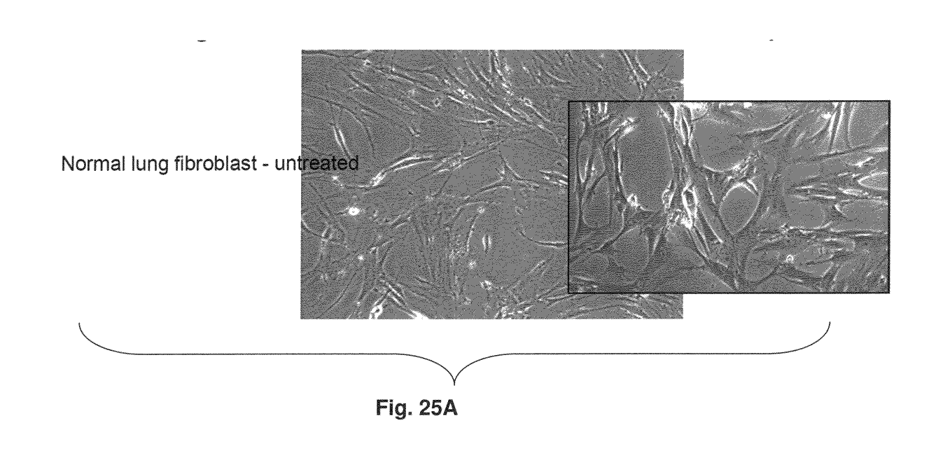

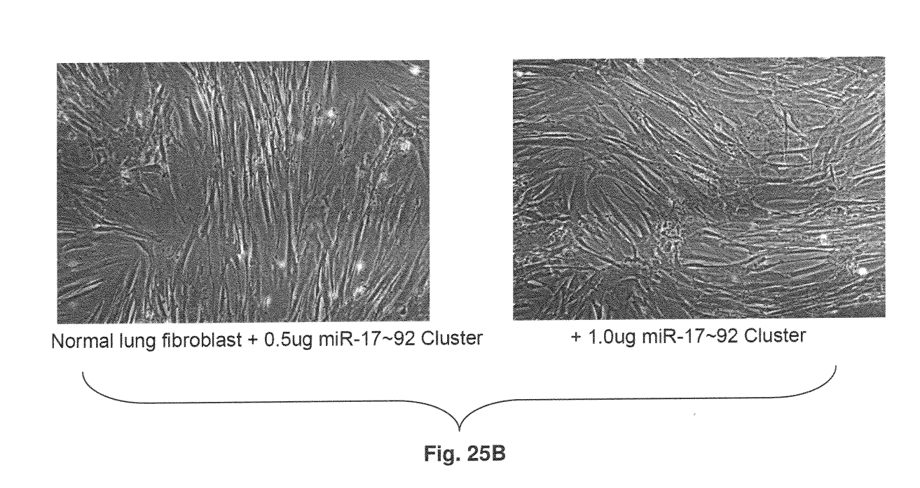

Overexpression of the miR-17˜92 cluster in normal lung fibroblasts does not alter their phenotype, as shown in FIGS. 25A-25B.

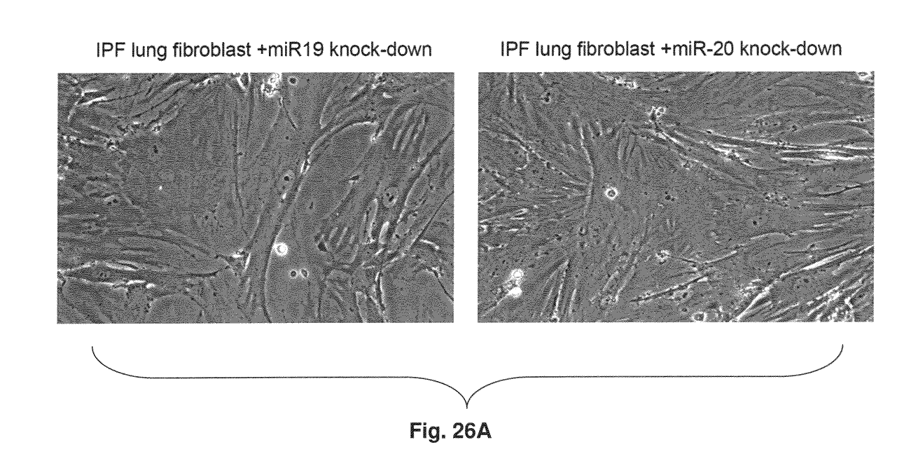

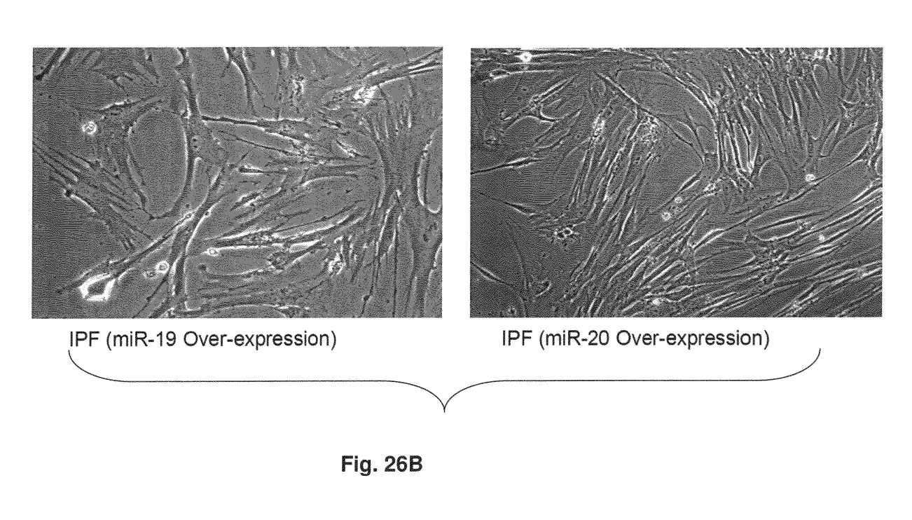

Overexpression but not knockdown expression of miR-19b or miR-20a induced phenotypic changes in IPF lung fibroblast cell lines, as shown in FIGS. 26A-26B.

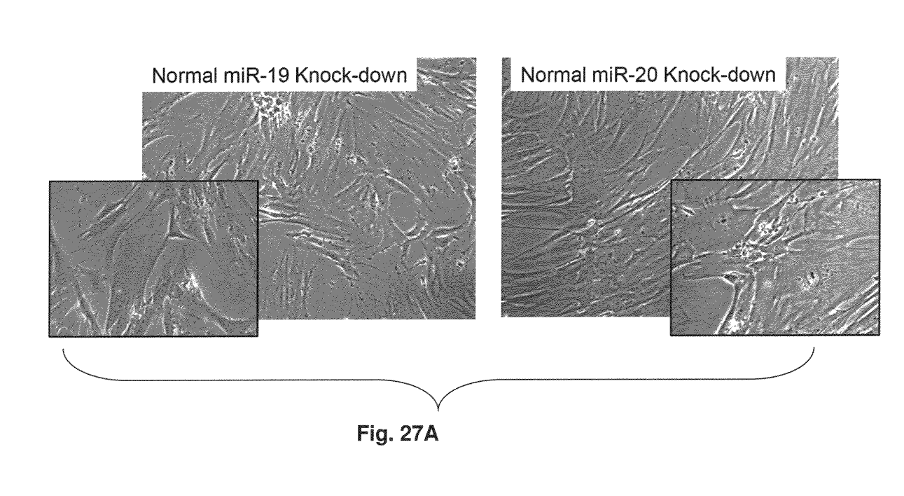

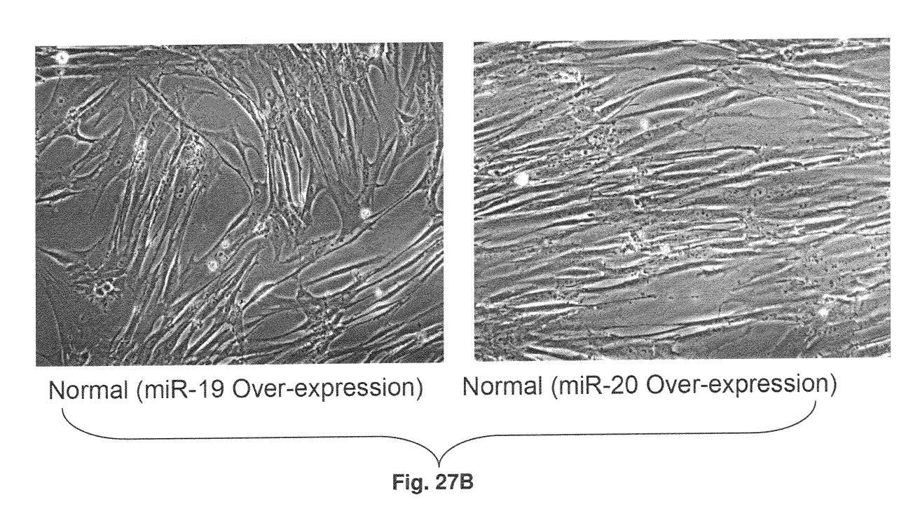

Knockdown expression of miR-19b or miR-20a induced normal lung fibroblast cell lines to become phenotypically similar to the IPF lung fibroblast cell lines, as shown in FIGS. 27A-27B.

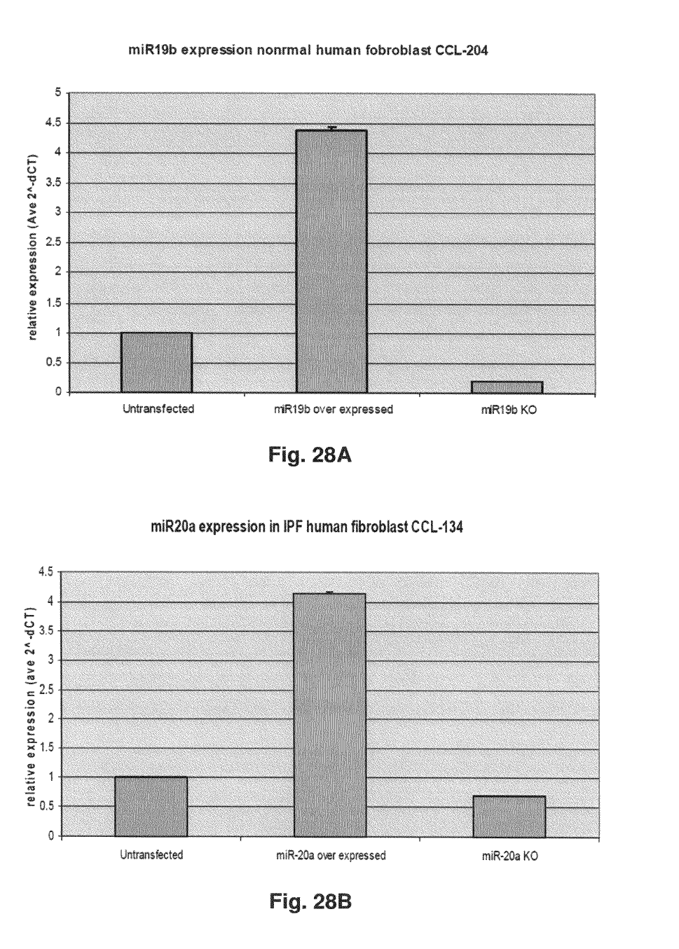

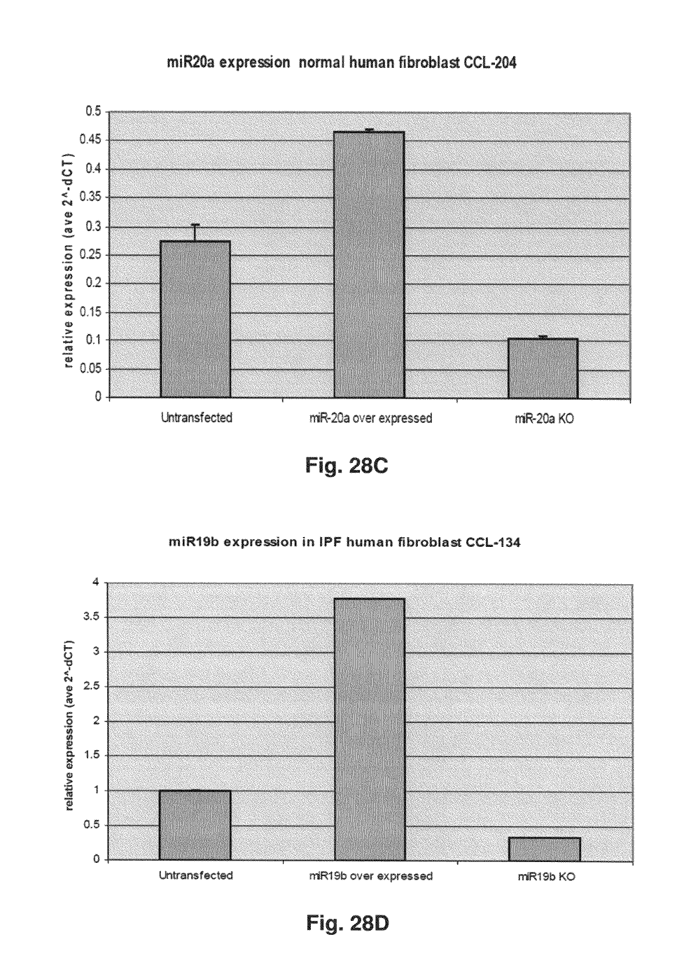

Confirmation of miR-19b and miR-20a expression in human lung fibroblasts after transfections is shown in FIGS. 28A-28D.

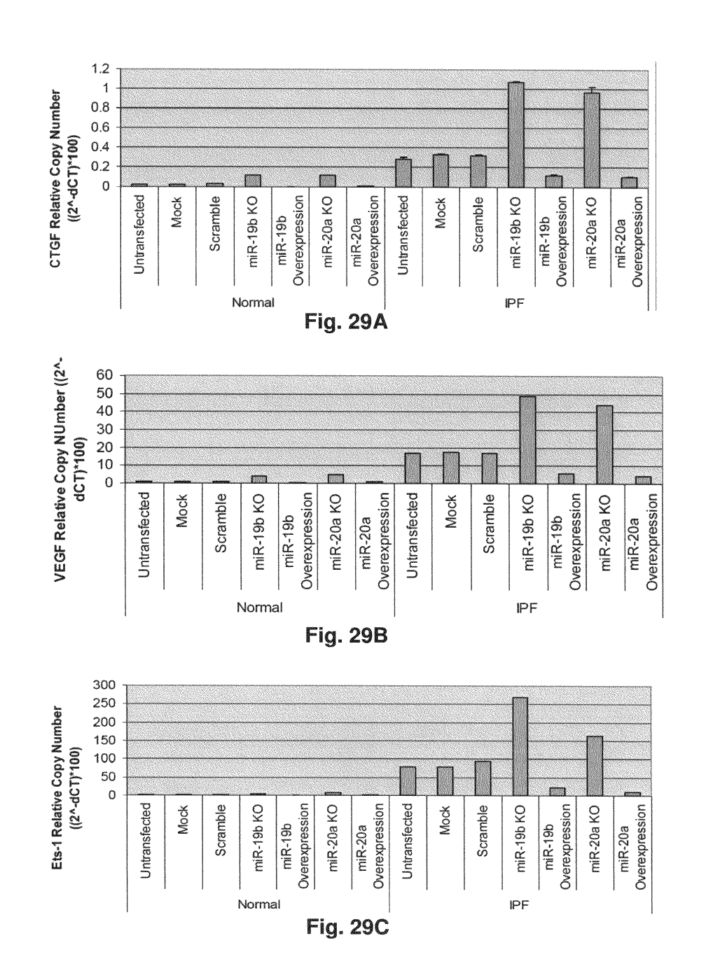

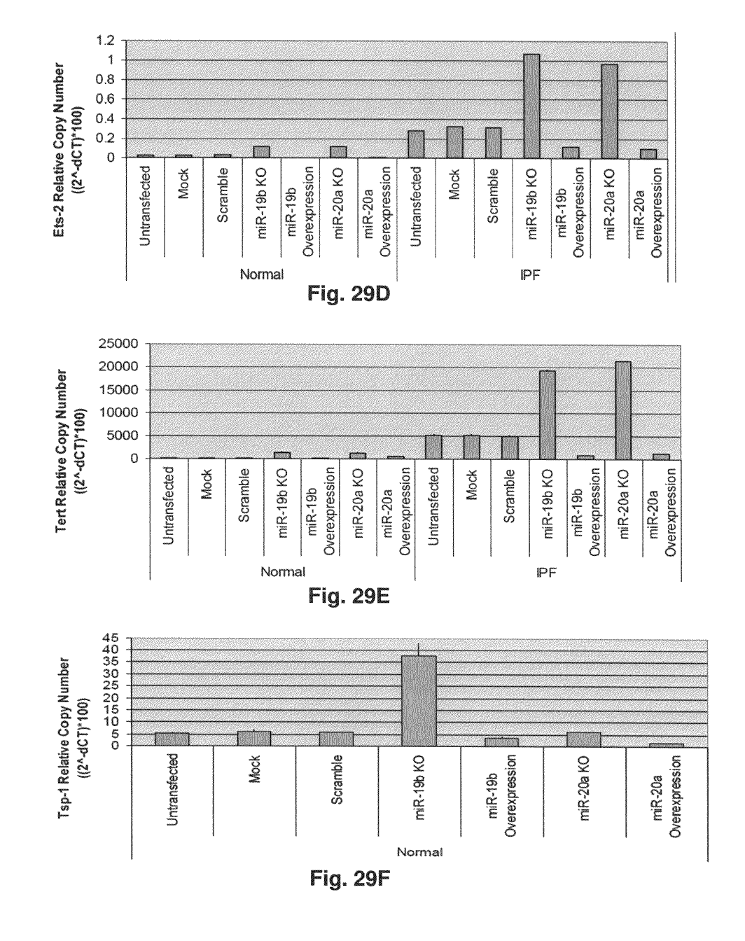

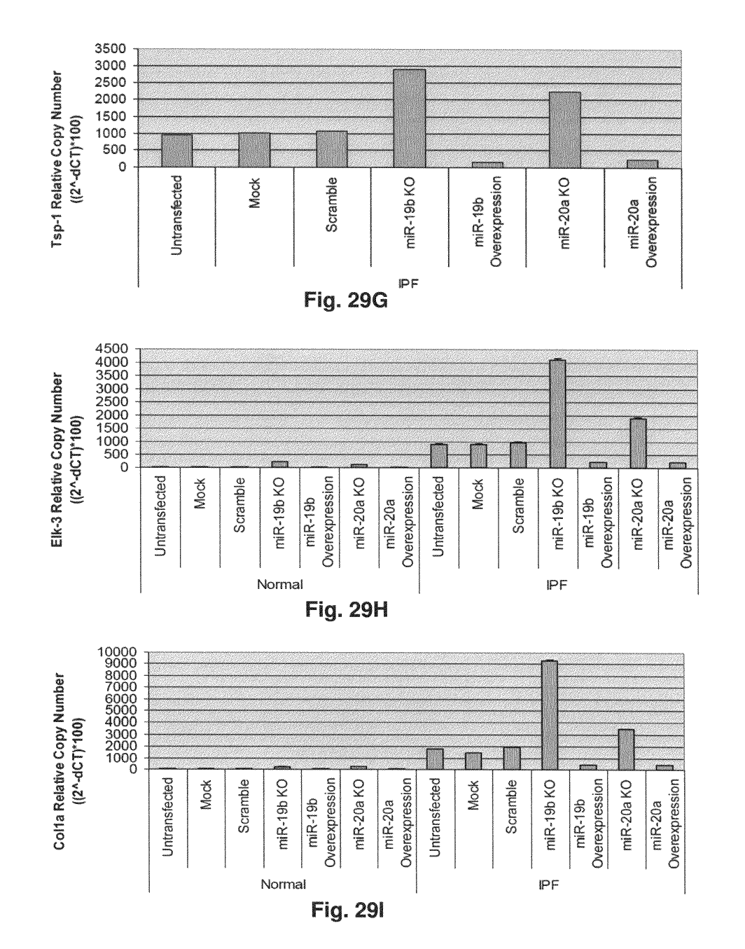

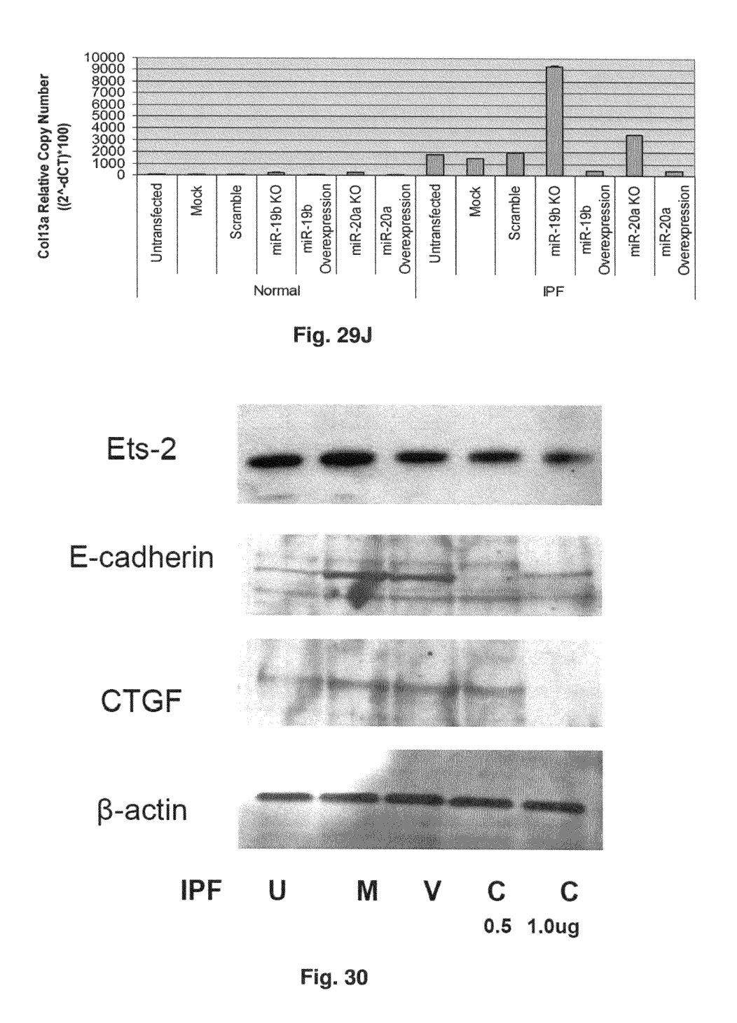

There is an increase gene expression in both normal and IPF fibroblast cell lines when expression of either miR-19b or miR20a is knockdown. In contrast, overexpression of these miRNAs resulted in decrease expression of the targeted genes. FIGS. 29A-29J show changes in gene expression.

An analysis of protein expression in IPF lung fibroblast cell lines transfected with miR-17˜92 cluster was conducted. Decreased protein expression following transfection of miR-17˜92 in IPF lung fibroblast cell lines was found, as shown in FIG. 30, where U=Untransfected, M=Mock transfection, V=Empty vector and C=miR-17-92 cluster.

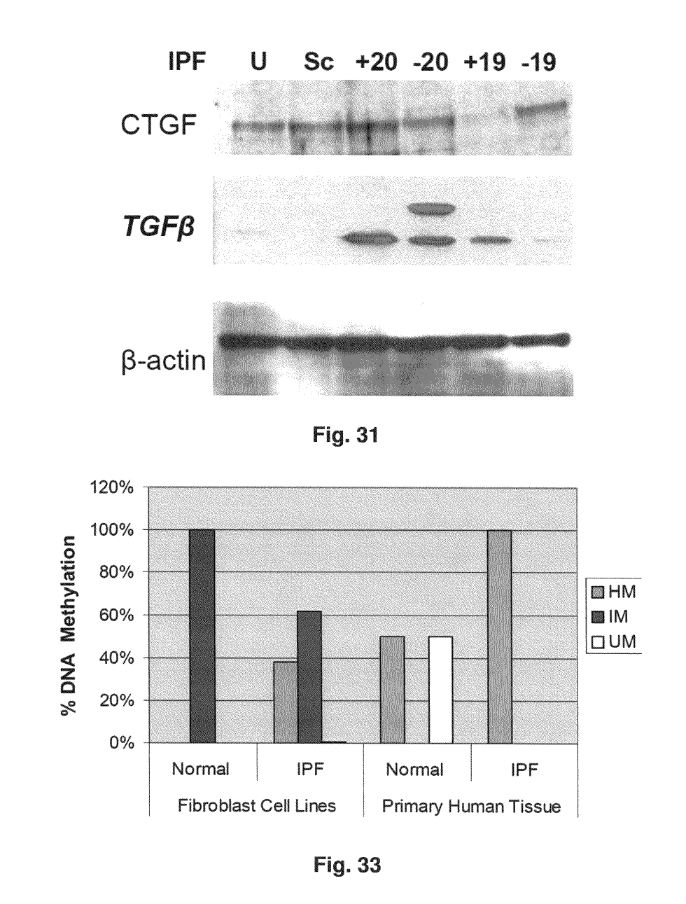

Decreased protein expression following transfection of either miR-19b or miR-20a in IPF lung fibroblast cell lines is shown in FIG. 31, where U=untransfected, Sc=scramble control, +20=miR-20a, 20=miR-20a antagomirs, +19=miR-19b, and 19=miR-19b antagomirs.

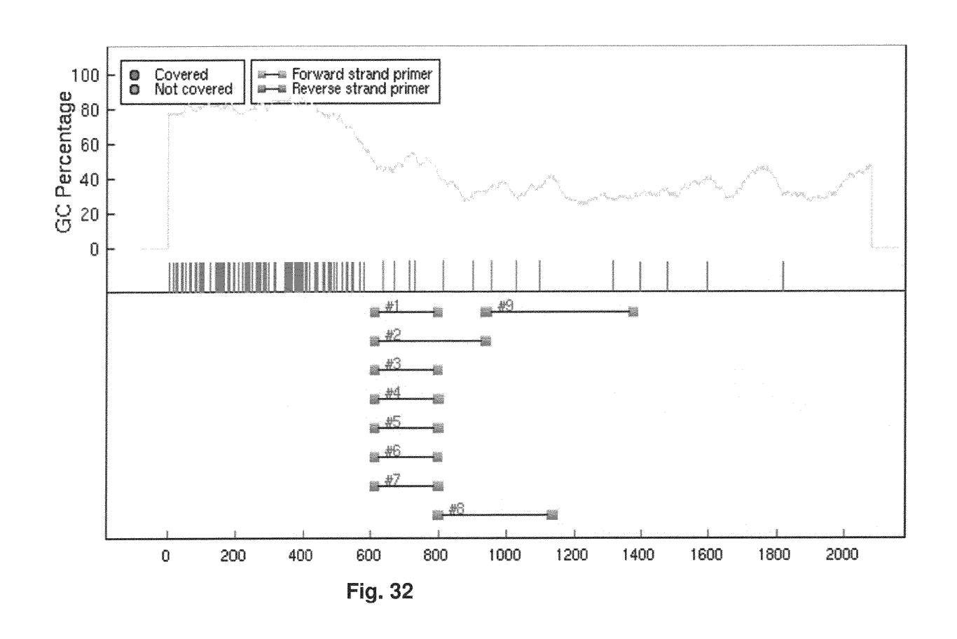

The location of CpG islands in the promoter of miR-17˜92 and primer sequences used for DNA methylation studies are shown in FIG. 32. The promoter of the miR-17˜92 cluster is rich in CpG islands, and it is now shown herein that the decrease in the expression of the cluster is due to epigenetic changes.

Increased DNA methylation of miR-17˜92 promoter in IPF tissue and fibroblast cell lines compared to normal tissue and cells is shown in FIG. 33.

### Example 8

### Epigenic Regulation by miRNA Expression in IPF

Primary Tissue Samples

De-identified tissue samples from IPF and COPD patients were acquired through the Lung Tissue Research Consortium. IPF and COPD samples were segregated into three FVC groups and two FVC groups, respectively.

Cell Culture and Treatments

Normal and IPF lung fibroblasts (American Type Culture Collection, Rockville, Md.) were cultured per instructions. Cells (2-3×10⁶) were treated with 0.5-2.0 μM of 5′-aza-2′-deoxycytidine for 24-72 hrs. Non-specific toxicity was measured. Fibroblasts (1×10⁶) were transfected using siPORTNeoFX transfection reagent for 24 hrs.

Bleomycin-Induced Pulmonary Fibrosis and 5′-aza-2′-deoxycytidine Treatment in Mice

C57/BL6 mice (Jackson Laboratory, Bar Harbor, Me.) were injected intraperitoneally (i.p.) with 0.035 U bleomycin/kg or vehicle (PBS). After two weeks, mice were injected (i.p.) twice weekly with endotoxin-free 0.156 mg/kg 5′-aza-2′-deoxycytidine or DMSO on alternating days in respect to bleomycin injections. Tissue and primary cells were isolated.

Quantitative Real-Time (qRT)-PCR Analyses

RNA was isolated and subjected to PCR analysis for miRNA and mRNA expression. For mRNA analysis, the endogenous controls GAPDH and CAP-1 were used for normalization. For miRNA analysis, several endogenous controls including miR-191, small nucleolar (sno) RNA, RNU38B, RNU48, snoRNA 202, and snoRNA 220 were evaluated for each experiment. The endogenous control with the most consistent Ct value and little variation was used for normalization as indicated.

Examination of DNA Methylation Patterns

DNA was isolated using Perfect Pure DNA Cultured Cell Kit (5 Prime Inc., Gaithersburg, Md.) or QIAamp DNA Mini Kit (Qiagen, Valencia, Calif.) from cells or tissue, respectively. DNA methylation of was analyzed using the Methyl-Profiler™ DNA Methylation qPCR Primer Assays (SABiosciences/Qiagen, Frederick, Md.) according to instructions

miRNA Co-Localization Studies

Antibodies recognizing AE 1/3, CD45 and CD31 were used to co-localize with miR-19b and miR-20a expression by in situ hybridization within fixed lung tissue. In situ hybridization was performed using 5′-digoxigeninlabeled LNA probes (1-2 pmol/P1) for either miR-19b or miR-20a.

Luciferase Reporter Experiments.

The 3′ UTR of DNMT-1 cloned into pGL-3 Luciferase reporter vector was provided by Dr. Muller Fabbri (The Ohio State University) and the 3′ UTR of Col13a in the pEZX-MT05 reporter vector was purchased from (GeneCopoeia Inc., Rockville, Md., USA). The miRNA recognition sites were mutated. Firefly and Renilla luciferase activities were measured consecutively in HEK 293 cells transfected with reporter vectors.

Statistical Analysis.

All data are expressed as the mean±S.D. One-way ANOVA was performed with SPSS16 (SPSS Inc. Chicago, Ill.), and JMP/SAS v9.1 software (SAS Institute, Inc., Cary, N.C.). Holm's method was used to adjust for multiplicity and control the overall Type I error rate at α=0.05. To test for outliers, normality test was performed and none were identified. Statistical significance was defined as p<0.05.

Reagents

Media and cell culture supplements, as well as reagents for RNA isolation, cDNA generation and miRNA analysis were purchased from Life Technologies (Carlsbad, Calif.), unless specified. FBS was obtained from Atlanta Biologicals, Inc. (Lawrenceville, Ga.). Antibodies for Western blot analysis were obtained from Santa Cruz Biotechnology, Inc. (Santa Cruz, Calif.). All other reagents including 5′ aza-2′-deoxycytidine (5-Aza) were purchased from Sigma-Aldrich (St. Louis, Mo.) unless otherwise indicated.

Cell Culture and Treatment

Normal lung fibroblasts (Cat#CCL-204) were cultured in Dulbecco's modified Eagle's medium (DMEM) supplemented with 10% FBS, 1% Penicillin/Streptomycin (100 U/100 mg/ml) and 1% sodium pyruvate. IPF lung fibroblasts (Cat#CCL-134) were cultured in Ham's F12K medium containing 15% FBS and 1% Penicillin/Streptomycin. All cells were incubated at 37° C. at 5% CO₂. Cells were cultured up to 10 passages.

Fibroblasts (1×10⁶) were transfected with 0.5 or 1.0 pg miR-17˜92/pcDNA3.1 expression vector (Dr. Joshua Mendel, Johns Hopkins University, Baltimore, Md.), pre-miR (50 nM) or antagomir (75 nM) using siPORTNeoFX transfection reagent for 24 hrs. Empty vector or mock-transfected cells served as controls. Transfection efficiency was 65%. To visualize actin filaments, cells were stained with 5 pg/ml rhodamine-conjugated phalloidin (Sigma-Aldrich, St. Louis, Mo.).

Primary Tissue Samples

De-identified tissue samples were acquired through the Lung Tissue Research Consortium (LTRC #07-99-0006), Lifeline of Ohio and the Cooperative Human Tissue Network (CHTN). The use of these samples was approved under IRB protocol #2007H0002. IPF and COPD patient samples were obtained from the LTRC. Samples obtained from patients with IPF were segregated into three FVC groups: severe (<50% FVC), moderate (50-80% FVC), or mild (>80% FVC, least severe breathing impairment). COPD samples were stratified in two FVC groups: <50% FVC and 50-80% FVC. Control samples consisted of tissue rejected for lung transplantation and control normal adjacent tissue from lung biopsies from Lifeline of Ohio and the CHTN, respectively.

Quantitative Real-Time (qRT)-PCR Analyses

Examination of the pre- and mature miRNAs for the miR-17˜92 cluster was performed according to manufacturer instructions. For analysis of each individual miRNA, RNA (100 ng) was converted to cDNA by priming with looped primers specific for each miRNA according to manufacturers' instructions. Several endogenous controls including miR-191, small nucleolar (sno) RNA, RNU38B, RNU48, RNU6, snoRNA 202, and snoRNA 220 were evaluated for each experiment. For quantitative (q)RT-PCR expression for mRNA, cDNA was synthesized from 5 pg RNA by oligo-dT primer and superscript II then amplification was performed with the SYBR green-based detection system using standard conditions. Commercial primer sets for all genes were obtained from SABiosciences/Qiagen (Frederick, Md.) except the housekeeping gene adenylyl cyclase-associated protein-1 (CAP-1). For normalization of expression levels, GAPDH and CAP-1 primers were used.

The reactions were performed using an ABI PRISM Sequence Detector 7700 (1). All miRNA and mRNA expressions were quantified using 2A-L Ct method. For normalization of each experiment, the endogenous control with the most consistent Ct value with little variation between samples was selected. In addition, to serve as an endogenous control the Ct value was required to have an average value of 20±1.5. In many cases, Ct values were relatively different among these housekeeping controls. However, when possible, the average Ct value of several endogenous controls was used.

Bleomycin-Induced Pulmonary Fibrosis and 5′-aza-2′-Deoxycytidine Treatment in Mice

The Ohio State University Institutional Laboratory Animal Care approved all animal experimental procedures and Use Committee (ILACUC) under protocol #2009A0124 and the animals were handled in accordance with their guidelines. Pulmonary fibrosis was induced by intraperitoneal (i.p.) injection with 0.035 U bleomycin/kg or vehicle control (PBS) twice weekly for 4 weeks. Bleomycin-treated mice received 5′-aza-2′-deoxycytidine two weeks after the initiation of bleomycin. Since inflammation and fibrosis begins after two weeks of bleomycin injections, the two-week treatment was chosen to target the lung during the fibrotic process. The 5′-aza-2′-deoxycytidine treatment was on alternating days in respect to bleomycin injections. The drug was administered via i.p. injection twice weekly at 0.156 mg/kg. This regimen was used to ensure continual incorporation of the drug into DNA while limiting toxicity to the mice based on an experimental dose curve (data not shown). PBS and DMSO vehicle treated mice served as a control, as well as bleomycin only treated mice. One week following the last injection, mice were sacrificed, and the lungs were inflated at 20 cm pressure and processed. Briefly, the left lobe of the lung was placed in 10% formalin for immunohistochemical (IHC) processing for trichrome and H&E. The right lobes were snap frozen in liquid nitrogen for DNA, RNA and protein extraction. Slides were photographed using an Olympus microscope equipped with a digital camera (Center Valley, Pa.). Total pixels were counted per stain using Adobe Photoshop CS2 software (San Jose, Calif.).

Primary lung fibroblasts and epithelial cells were purified. Briefly, lungs were minced and digested with collagenase (0.15% collagenase I, 160 U/ml hyaluronidase, 1 mg/ml hydrocortisone and 10 mg/ml insulin with penicillin and streptomycin) then incubated in a 5% CO₂ incubator overnight at 37° C. Collagenase was then neutralized with 10% FBS-DMEM medium and the digested tissue was subjected to gravity sedimentation. Pellets were washed three times to collect epithelial organoids and cultured to obtain epithelial cells. To enrich for fibroblast cells, the supernatant was subjected to four additional gravity sedimentation and the supernatant containing fibroblasts cultured. Cell purity was validated by fluorescent immunostaining by flow cytometry using cytokeratin and vimentin to detect epithelial cells and fibroblasts, respectively. The purified epithelial cells and fibroblasts were also subjected to qRT-PCR analysis for the expression of keratin and collagen 1, respectively.

Immunohistochemistry and In Situ Hybridization

Immunohistochemistry and in situ hybridization were used to co-localize miR-19b and miR-20a expression within the lung tissue. Frozen lung tissue was thawed over 48 hrs, fixed in 10% buffered formalin for 8-15 hrs, then washed with 70% ethanol followed by three washes with PBS at room temperature, paraffin-embedded and sectioned. Serial sections from the same block were used for further analysis. The Benchmark LT automated system (Ventana Medical Systems, Tucson, Ariz.) was used according to the manufacturer's specifications. Optimal conditions were determined for detection of the antibodies as protease pretreatment for AE 1/3 (DAKO, Carpinteria, Calif.) diluted 1:100; and cell conditioning 1 (CC1) solution (Ventana Medical Systems) for antigen retrieval with no dilutions for both CD45 and CD31 (Ventana Medical Systems) ready-to-use antibodies. For alpha smooth muscle actin (a-SMA, Ventana Medical Systems), the antibody was diluted 1:1. The antigens were detected with the Ultraview Universal DAB or Fast Red system from Ventana then counterstained with hematoxylin. The negative controls included omission of the primary antibody and the internal control of cells known to be negative for the targets.

In situ hybridization was performed. Briefly, after protease digestion, the 5′-digoxigeninlabeled LNA probes (1-2 pmol/μl) for either miR-19b or miR-20a were incubated with the tissue section at 60° C. for 5 mins, and then hybridized for 15 hrs at 37° C. The slides were then washed in 0.2×SSC and 2% bovine serum albumin at 40° C. for 5 mins, then incubated with antidigoxigenin-alkaline phosphatase conjugate (1:200 dilution) for 30 mins at 37° C. The miRNAs were visualized by alkaline phosphatase reaction with nitroblue tetrazolium and 5-bromo-4-chloro-3-indolyl phosphate (NBT/BCIP) chromogen system (Roche, Nutley N.J.). In some cases, the nuclei were counterstained with fast red. Negative controls included the omission of the tagged probe from the probe cocktail and the use of a scrambled probe. As a positive control, miR-let-7c was used.

To interpret the co-localization signal, the Nuance multi-spectral imaging system (Caliper Life Sciences, Inc., Hopkinton, Mass.) was used. This microscope/computer-based interface separates the colorimetric based signal for each of the different colors of the light spectrum, and then converts these color-based signals to fluorescence-based signals. This readily allows fluorescence-mixing combinations to determine if a given cell is co-expressing the targets of interest.

Phalloidin Staining.

Cells were grown on cover slips placed in 6 well plates, after reaching 50-60% confluency, cells will be transfected and incubate for 24 hrs. Cells were fixed in 3.7% formaldehyde in PBS for 10 mins at room temperature. Cells were permeabilized with Triton X-100 in PBS for 10 mins at room temperature then incubated with rhodamine-conjugated phalloidin and incubated for 30 mins in the dark and washed with PBS for 30 mins. The cells were then incubated with DAPI (0.5 μg/ml) for 10 mins and washed again. Using an Olympus inverted fluorescence microscope, the actin filaments were visualized. Quantification of red fluorescence intensity was performed using Adobe Photoshop software and reported as a percent in respect to DAPI.

Luciferase Reporter Experiments.

The 3′ UTR of DNMT-1 (provided by Dr. Muller Fabbri, The Ohio State University) was cloned into the pGL-3 Luciferase reporter vector. Notably, the miRNA recognition sites overlap for miR-17, miR -20a, miR-19b and miR-92a. Thus, a mutant 3′ UTR lacking the recognition site for these four miRNAs was generated by deleting 20 nucleotides using the QuikChange XL-site directed Mutagenesis Kit (Aligent Technologies, Inc., Santa Clara, Calif.). Wild-type (WT) and mutant inserts were confirmed by sequencing.

The HEK 293 cell line was co-transfected in six-well plates using Lipofectamine 2000 (Life Technologies) with 0.4 pg of the firefly luciferase reporter vector, 10 nM each miRNA (Life Technologies) and 0.08 μg of the control vector (pRL-TK) containing Renilla luciferase (Promega Corp., Madison, Wis.) for normalization. Firefly and Renilla luciferase activities were measured consecutively by using the dual luciferase assays (Promega Corp.) 24 hrs after transfection using an VectorTm×3 plate reader (Perkin Elmer life, Shelton, Conn., USA). Experiments were performed in duplicate from eight independent transfection experiments. The wild-type 3′UTR of CoI13a1 was mutated to generate a mutant 3′ UTR of Col13a.

Both the wild-type and mutated sequences were cloned into the pEZX-MT05 reporter vector downstream of the secreted Gaussia luciferase (GLuc) reporter gene driven by SV40 promoter for expression in mammalian cells (GeneCopoeia Inc., Rockville, Md., USA). A secreted Alkaline Phosphatase (SEAP) reporter driven by a CMV promoter was also cloned into the vector to serve as the internal control. The dual-reporter vector system enables transfection-normalization for accurate across-sample comparison. The HEK 293 cells were then co-transfected with 2.5 pg of plasmid and 20 nM of each miRNA of the cluster, positive miRNA control (GeneCopoeia Inc.) or a scrambled negative control (Ambion). Cells were harvested 24 hr post transfection and Firefly and Renilla luminescence were measured using Luc-Pair luciferase assay (GeneCopoeia Inc.) according to the manufacturer's instructions.

miRNA Prediction Analysis.

Several online miRNA target prediction software programs were used to identify targets of the miR-17˜92 cluster including TargetScan, miRanda, PicTar, miRBase, miRNAMap, miRGen, and EMBL Microcosm Targets. For databases that generate a score based on their algorithms, a cut-off was applied accordingly. Upon comparing the predicted targets, a potential gene target for a given miRNA was indicated as long as it was predicted by at least two databases.

miR-17˜92 Expression is Decreased in Lung Tissue from Patients with IPF

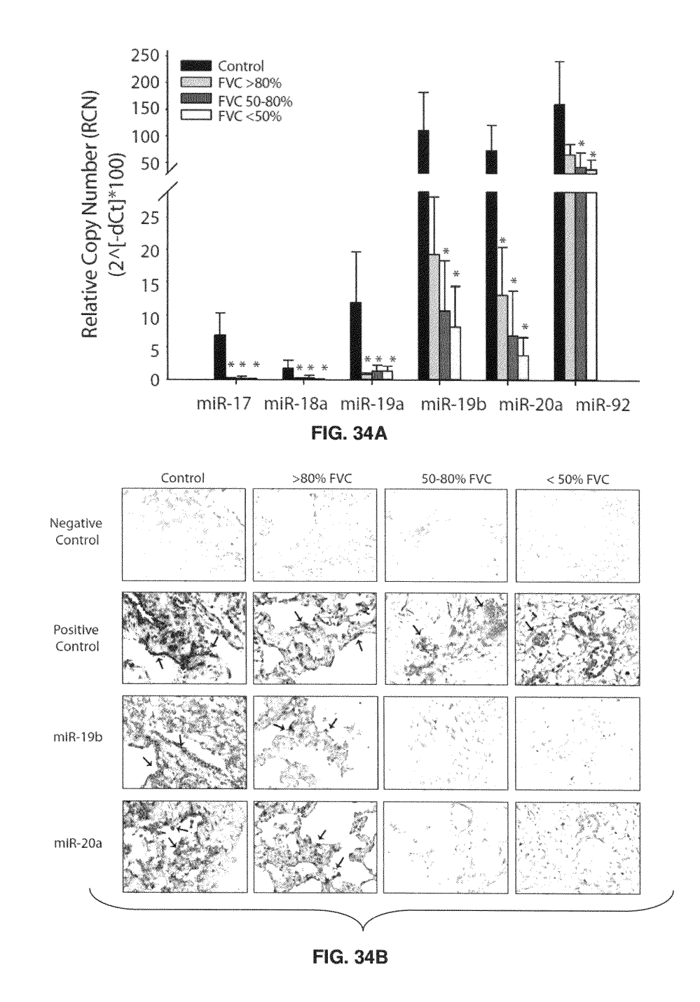

The IPF lung tissue were stratified into severity groups based on forced vital capacity (FVC): group 1: FVC <50% (severe); group 2: FVC 50-80% (moderate); and group 3: FVC>80% (mild). IPF lung tissue samples demonstrated reduced expression of pre-miRNAs (data not shown) and mature miRNAs encoded by the miR-17˜92 cluster compared to control lung tissue samples including pathologically normal lung tissue adjacent to lung cancer (FIG. 34A). Based on miRNA target prediction software programs, the miR-17˜92 cluster was predicted to target several fibrotic genes including collagen1 (Col1a1), transforming growth factor (TGF)-β, and metalloproteinases (MMPs) (Table 1).

TABLE 1
miRNAs that regulate genes expression involved in IPF

| Genes | miRNAs | | | | |
|---|---|---|---|---|---|
| COL13A1 | miR-19a,b | miR-18a,b | miR-29a,b | miR-377 | miR-544 |
| COL1A1 | miR-92a,b | miR-19a,b | miR-29a,b,c | miR-224 | miR-196a |
| CTGF | miR-18a,b | miR-19a,b | miR-559 | miR-600 | miR-377 |
| VEGF | miR-17 | miR-20a | miR-106a,b | miR-93 | miR-363 |
| TGFβ | miR-17 | miR-19a,b | miR-20a | miR-29a,b | miR-34b,c |
| TSP-1 | miR-17 | miR-18a,b | miR-19a,b | miR-20a | miR-34a,c |
| MMP-1 | miR-20a | miR-19a,b | miR-29a,c | miR-190b | miR-22 |
| MMP-9 | miR-19a,b | miR-92a,b | miR-581 | miR-34a,c | miR-223 |
| MMP-7 | miR-17 | miR-18a,b | miR-34b,c | miR-106b | miR-122 |

Abbreviations:
Collagen (Col),
Metalloproteinases (MMPs),
Vascular Endothelial Growth Factor (VEGF),
Connective Tissue Growth Factor (CTGF),
Transforming Growth Factor (TGF)-E, and
Thrombospondin (TSP)-1.

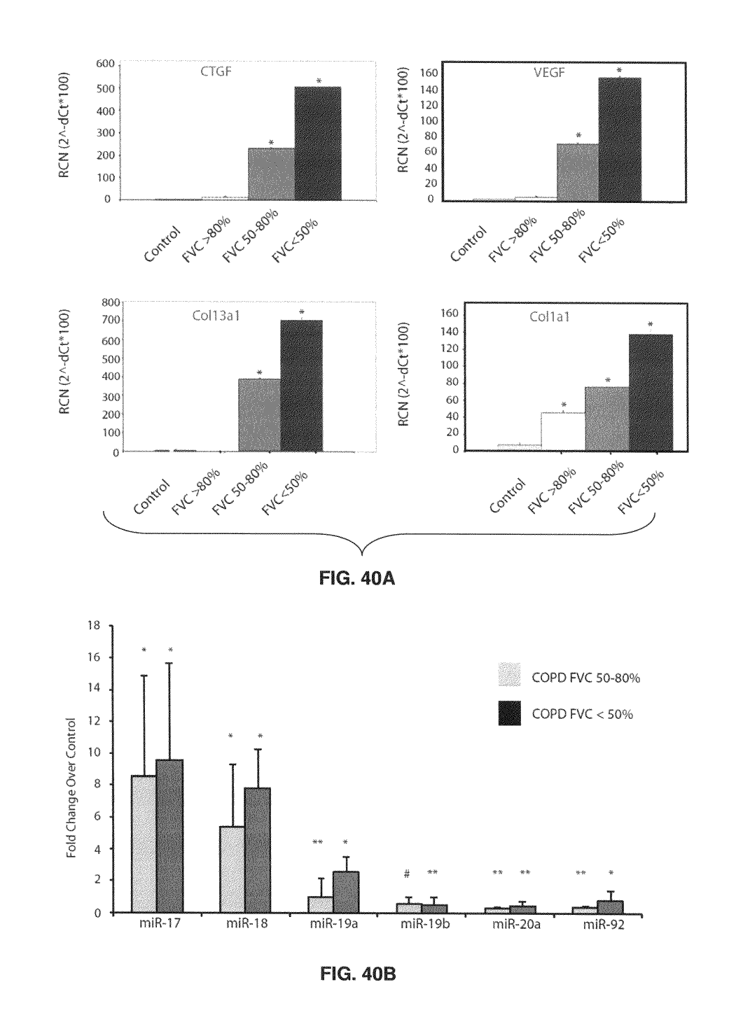

The expression of the above mRNA targets of the miR-17˜92 cluster was increased in lung tissue from IPF patients (FIG. 40A).

To determine if similar changes occurred in lung tissue from patients with other chronic lung diseases, the miR-17˜92 cluster expression in lung tissue from patients with chronic obstructive pulmonary disease (COPD) was examined. In contrast to IPF lung tissue, a significant elevation of the miRNAs in lung tissue from COPD patients was observed, as compared to control tissue samples (FIG. 40B), showing that reduced lung miR-17˜92 cluster expression was not a non-specific feature of chronic lung disease.

Detection of miRs-19b and miR-20a by In Situ Hybridization

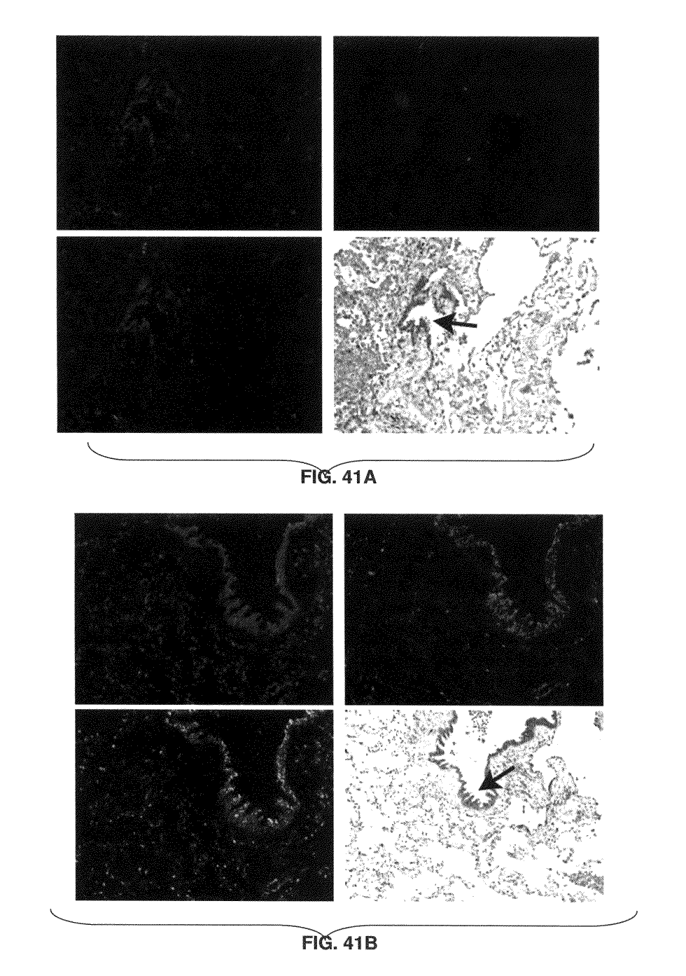

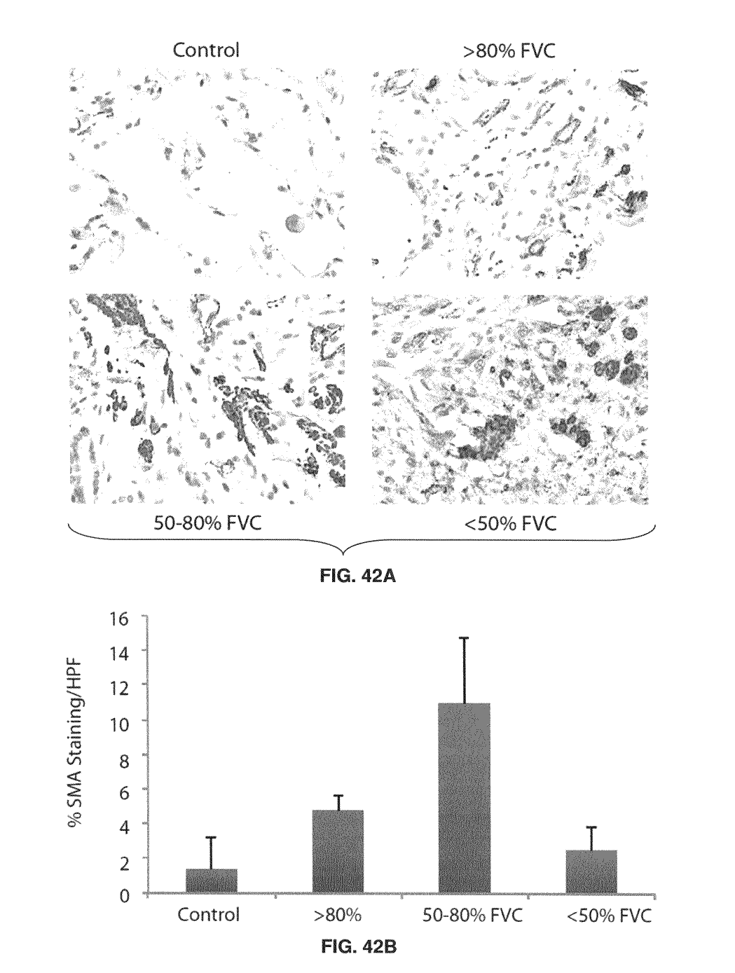

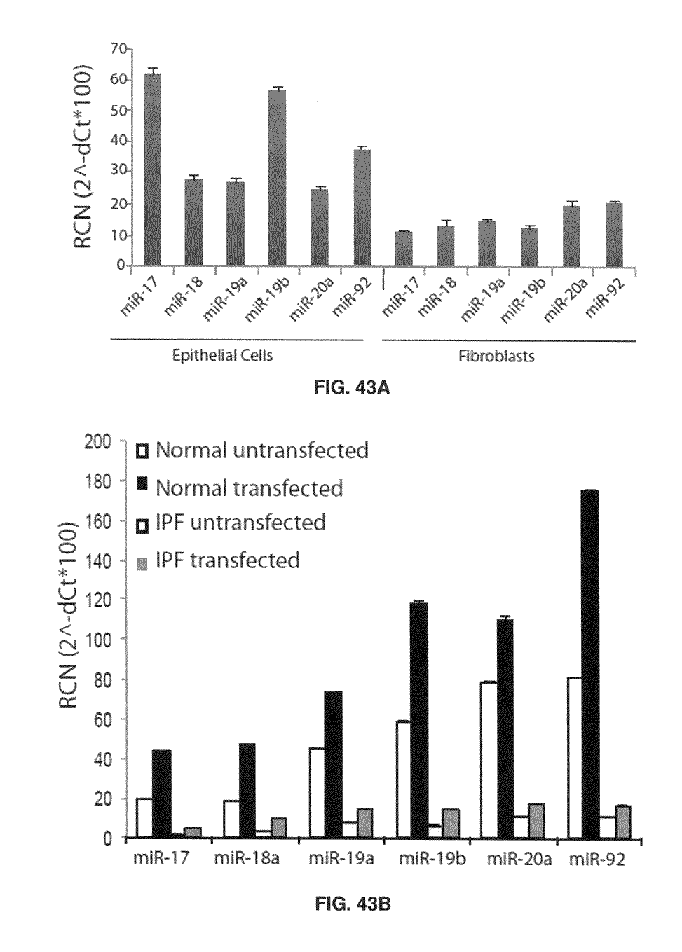

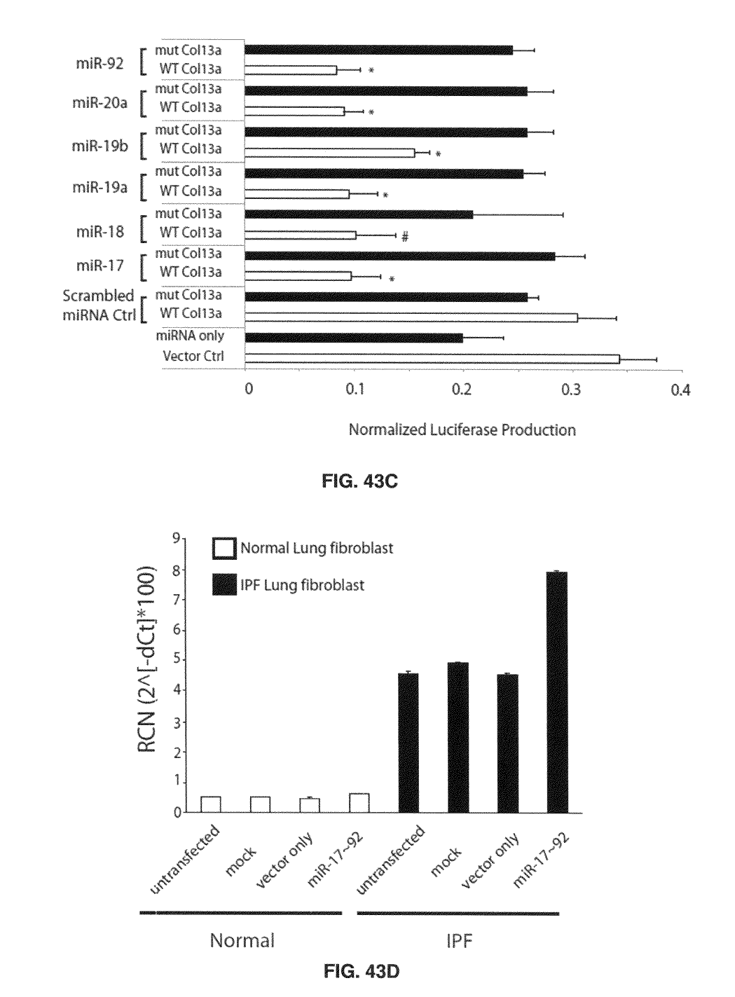

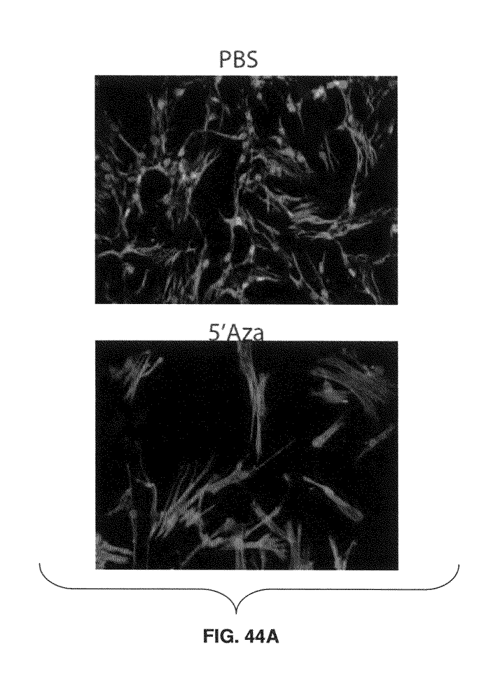

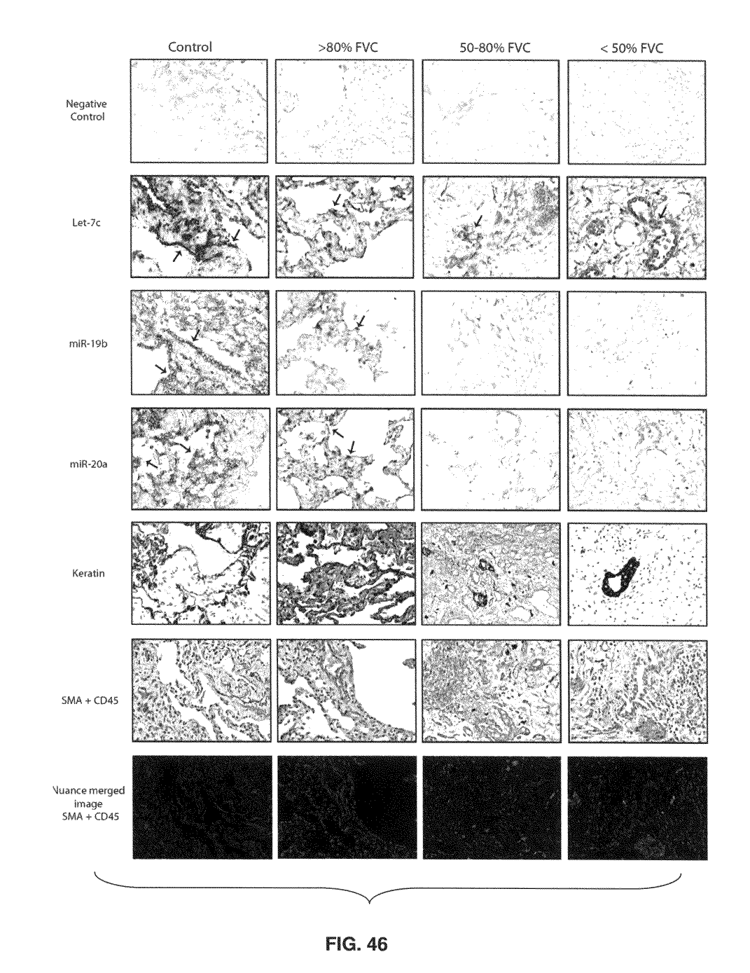

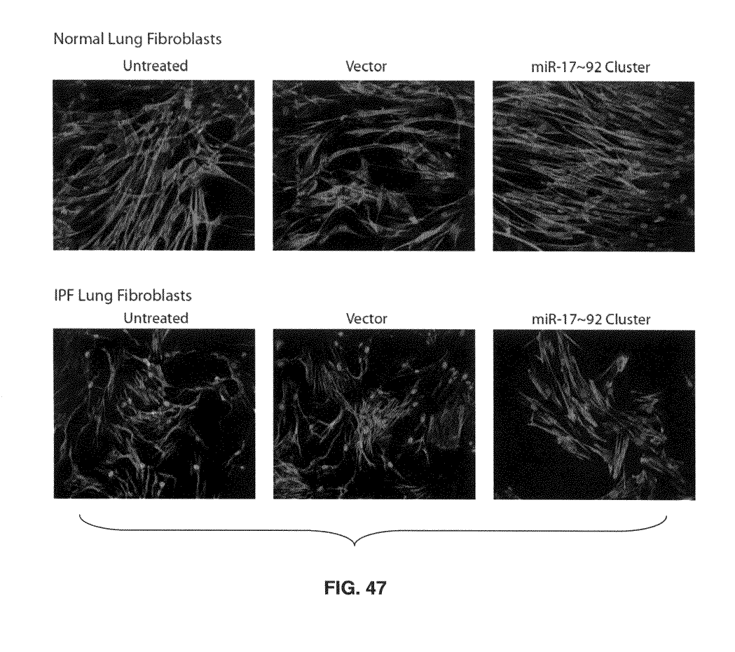

Members of the miR-17˜92 cluster, including miRs-19b and -20a, were assessed in normal lung tissue by in situ hybridization. miR-19b expression co-localized to lung epithelial cells expressing AE1/3 cytokeratin (FIG. 41). In IPF lung tissue, miR-19b and miR-20a were nearly undetectable in lung tissue from patients with moderate (50-80% FVC) and severe (<50% FVC) lung disease sections staining positive for alpha smooth muscle actin (D-SMA) and CD45 (FIG. 42 and FIG. 46). There was no non-specific staining of miRNAs in tissue stained with scrambled probes. A probe recognizing let-7c miRNA was used as a positive control and was similarly expressed among all tissue samples. miRNAs from the miR-17˜92 cluster, including miR-19b and miR-20a were expressed in freshly isolated lung epithelial and fibroblast cells from murine lung tissue (FIG. 43A).

Introduction of the miR-17˜92 Cluster in IPF-Derived Lung Fibroblasts Results in Molecular and Phenotypic Changes

In IPF, fibroblast activation leads to ECM deposition. The miR-17˜92 expression was analyzed in human normal and IPF lung fibroblast cell lines. Similar to IPF lung tissue, miR-17˜92 expression was decreased in IPF fibroblast cell lines compared to normal lung fibroblast cell lines (FIG. 43B).

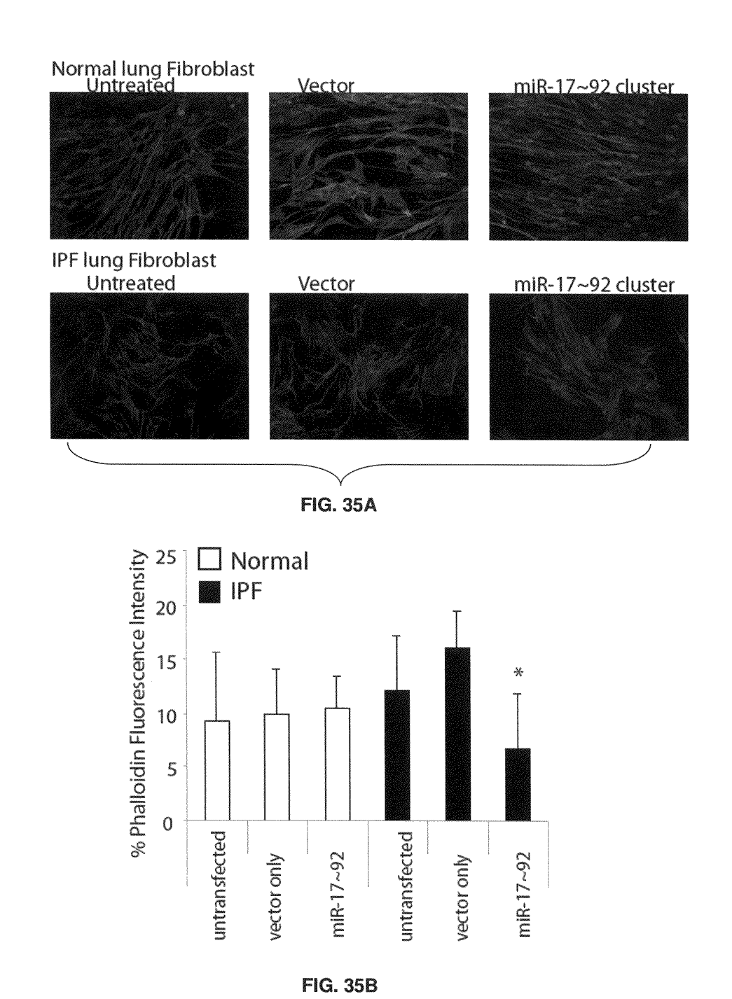

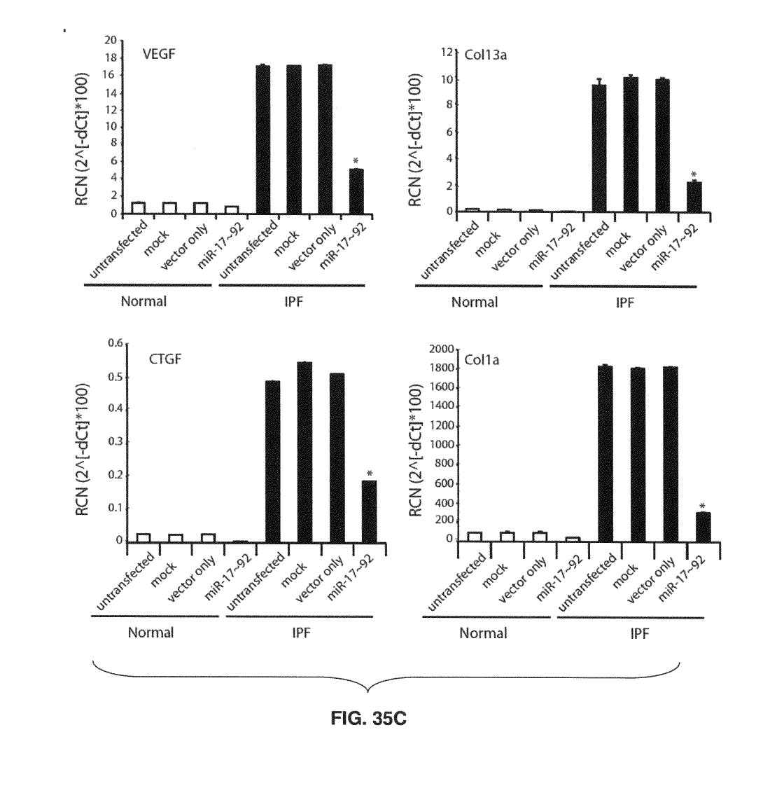

Gene profiles in IPF lung tissue are consistent with EMT and wound repair. Wound repair involves fibroblast migration to areas of damage and cells undergoing EMT gain filopodia as they assume fibroblast phenotypes. After transfecting miR-17˜92 in IPF and normal fibroblast cell lines, cell morphology, target gene and miR-17˜92 cluster expression were assessed (FIG. 35 and FIG. 43B). Cytoskeleton morphology as indicated by numerous filopodia and increased actin staining was different in untransfected and vector-transfected IPF lung fibroblasts compared to the normal lung fibroblast samples (FIG. 35A and FIG. 395). Introduction of the miR-17˜92 cluster into IPF cells reduced the actin staining to levels similar to the normal fibroblast. Overexpression of the miR-17-92 cluster did not alter the normal lung fibroblasts (FIG. 35B).

Transfection of the miR-17˜92 cluster in IPF fibroblasts reduced VEGF, CTGF, Col1a1 and

Col13a1 expression compared to untransfected, mock-transfected and vector-transfected cells (FIG. 35C). In contrast, gene expression was relatively unchanged in normal fibroblast cell lines transfected with the miR-17˜92 cluster compared to control samples. VEGF and CTGF are directly regulated by the miR-17˜92 cluster, while collagen Col1a is indirectly targeted by miR-17˜92 regulation of TGFβ. Since Col13a1 was a predicted target of the cluster, these results confirmed that it is directly regulated by the miRNAs contained in the miR-17˜92 cluster (FIG. 43C). As a negative control, HIF1α mRNA expression, highly expressed in lung tissue and not a predicted target of the miR-17˜92 cluster, was unchanged in miR-17˜92-transfected normal and IPF lung fibroblasts (FIG. 43D).

Altered DNA Methylation Patterns of the CpG Island in miR-17˜92 Cluster Promoter in IPF

Since c-MYC transcriptionally regulates miR-17˜92 cluster expression, its expression in lung tissue from patients with IPF was evaluated. No differences in mRNA or protein expression in lung tissue or fibroblasts from patients with IPF were found, thus showing alternative mechanisms accounted for decreased miR-17˜92 expression in IPF.

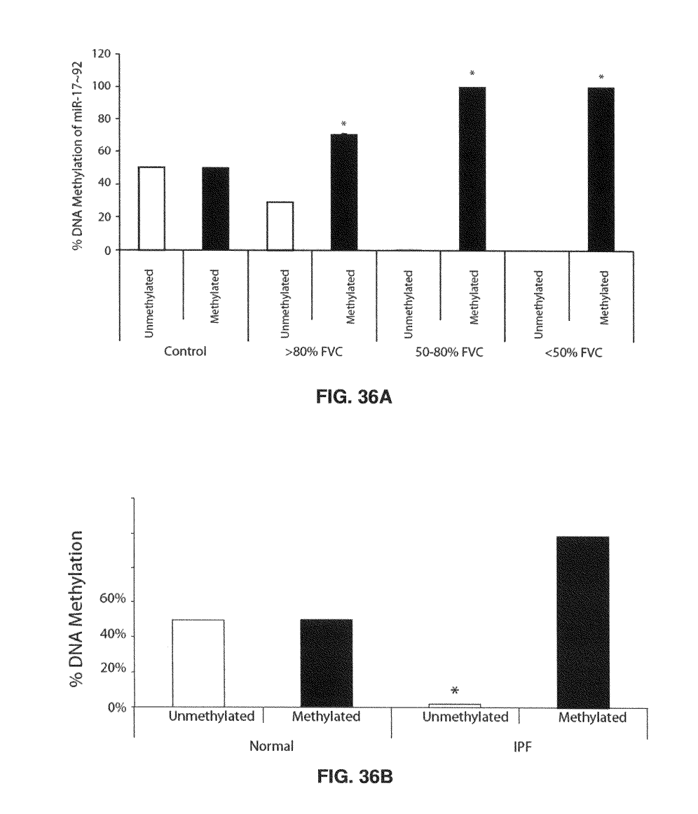

More than 80% of the miR-17˜92 promoter is heavily occupied with a large CpG island. This CpG island was aberrantly methylated in lung tissue from patients with IPF, reducing miR-17-92 cluster expression. In lung tissue from IPF patients, DNA methylation of the miR-17˜92 promoter was significantly increased compared to control lung tissue (FIG. 36A). No unmethylated DNA was detected for the miR-17˜92 promoter in lung tissue from patients with moderate (50-80% FVC) and severe (<50% FVC) IPF, while unmethylated DNA in the miR-17˜92 promoter was present in mild disease (>80% FVC).

A 20% increase in DNA methylation from baseline seen in mild disease samples was sufficient to reduce miR-17˜92 expression (FIG. 34A). In contrast, control lung tissue had an equal distribution of unmethylated and methylated DNA in the miR-17˜92 cluster promoter. Similarly, the miR-17˜92 promoter was methylated in lung fibroblasts derived from patients with IPF (FIG. 36B). Thus, these data show that silencing of the miR-17˜92 cluster occurred through DNA methylation of its promoter.

Inhibition of DNA Methylation in Lung IPF Fibroblasts Leads to Phenotypic and Genetic Changes

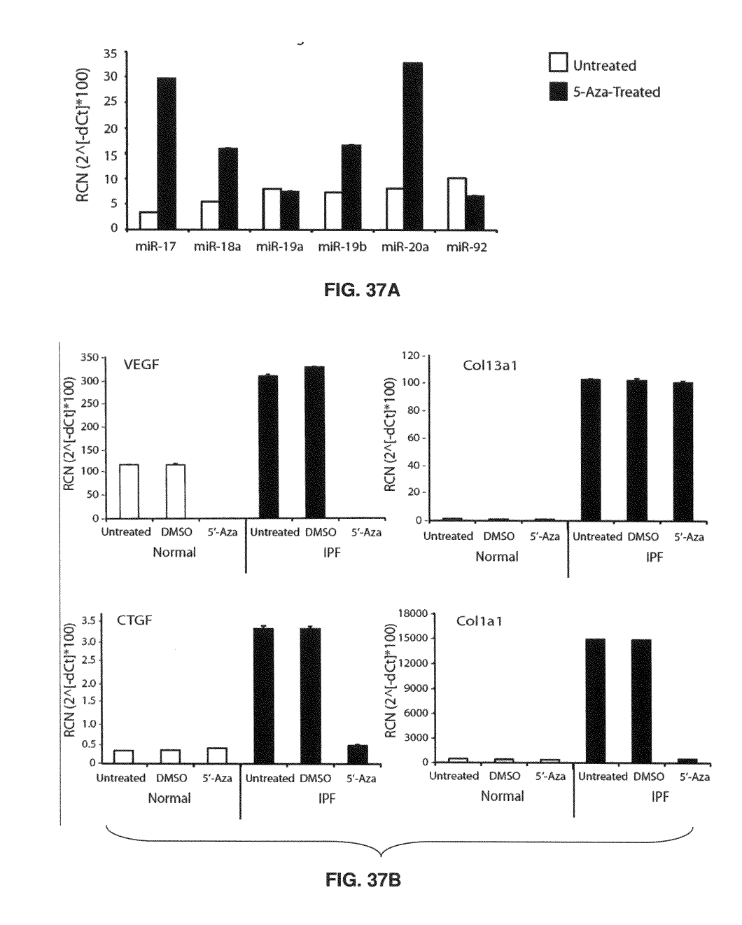

A compound that inhibits DNA methylation (5-'aza-2′-deoxycytidine) restored miR-17˜92 cluster expression in IPF fibroblasts. Treating IPF fibroblasts with 5′-aza-2′-deoxycytidine for 24 hours increased miR-17, miR-18a, miR-19b, and miR-20a expression (FIG. 37A). In contrast, there was no change in miR-19a and miR-92a expression.

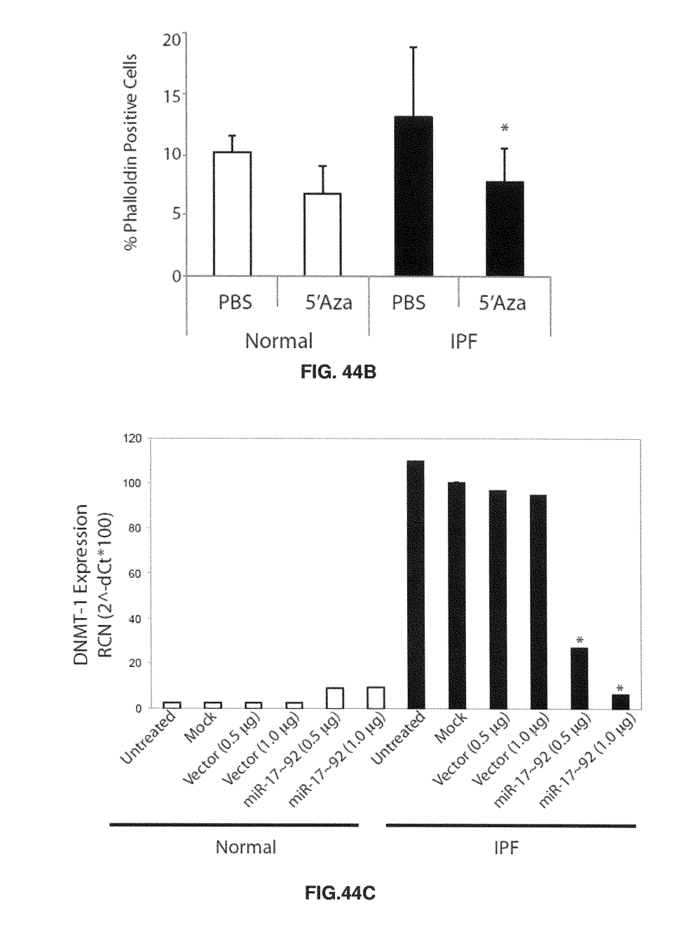

Gene expression was different between miR-17˜92-transfected and 5′-aza-2′-deoxycytidine-treated IPF fibroblasts (FIG. 37B). Notably, Col13a1 expression was decreased in miR-17˜92-transfected cells (FIG. 35C) but not in 5′-aza-2′-deoxycytidine-treated cells (FIG. 37B). Similar to cells transfected with the miR-17˜92 cluster (FIG. 35A), IPF fibroblasts treated with 5′-aza-2′-deoxycytidine exhibited a more normal phenotype (FIG. 44A).

Increased Expression of DNA Methyltransferase (DNMT)-1 in IPF

To define the mechanisms regulating DNA methylation of the miR-17˜92 cluster in IPF lung tissue, DNMT-1 expression was examined. DNMT-1 is the most abundant DNA methyltransferase in mammalian cells and is a predicted target of the miR-17˜92 cluster.

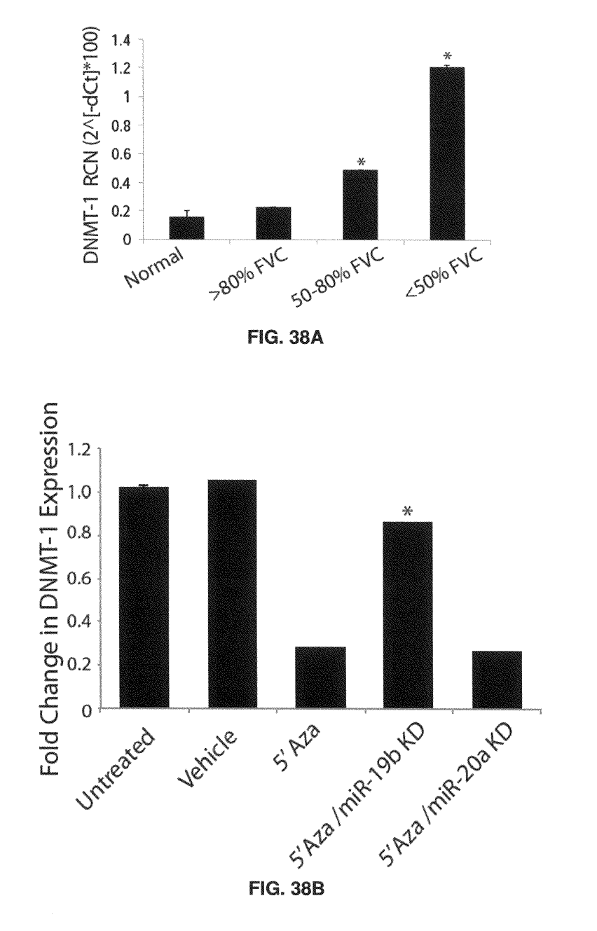

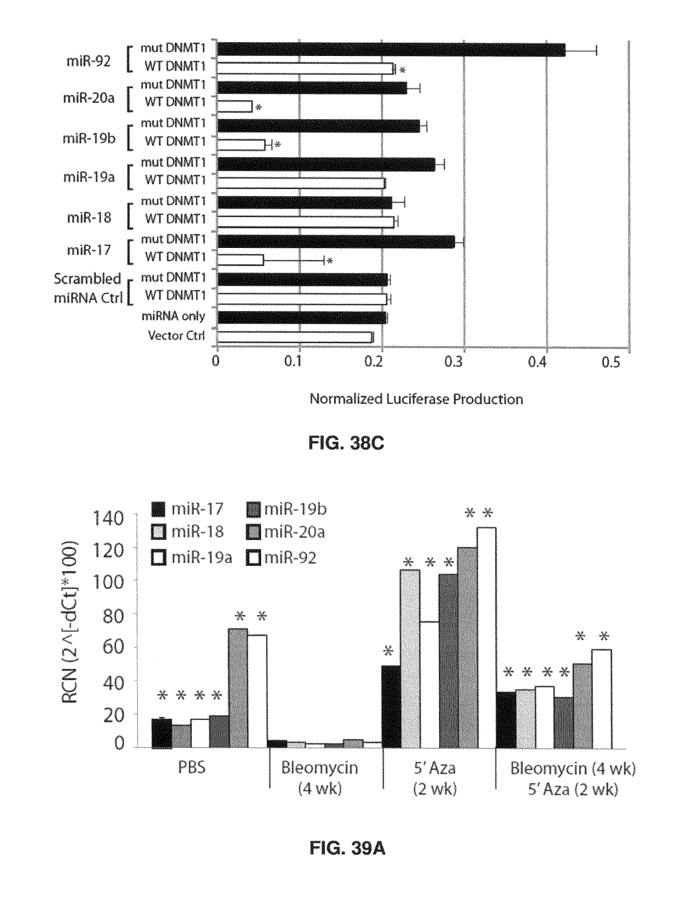

DNMT-1 mRNA expression increased as FVC declined in IPF lung tissue and inversely correlated with miR-17˜92 cluster expression (FIG. 38A). Similarly, DNMT-1 expression was elevated in IPF lung fibroblast cell lines compared to normal fibroblast cell lines (FIG. 44C). Introduction of the miR-17˜92 cluster in IPF fibroblast cell lines significantly decreased DNMT-1 expression compared to vector control-transfected samples (FIG. 44C). These data show that DNMT-1 contributed to the hypermethylation of the miR-17˜92 promoter region in IPF lung tissue.

Further, treatment of the IPF lung fibroblast cell line with 5′-aza-2′ deoxycytidine also decreased DNMT-1 expression (FIG. 44B).

IPF lung fibroblast cell lines were treated with 5′-aza-2′-deoxycytidine in the presence of an antagomir to either miR-19b or miR-20a to reduce miRNA-specific expression. After treatment with the antagomir to miR-19b, treatment with 5′-aza-2′-deoxycytidine no longer decreased DNMT-1 expression (FIG. 38B). In contrast, using the miR-20a antagomir did not alter DNMT-1 expression in the presence of 5′-aza-2′-deoxycytidine (FIG. 38B), indicating that miR-19b primarily regulated DNMT-1 expression.

DNMT-1 Expression is Directly Regulated by the miR-17˜92 Cluster

Seed sequences for miR-17, miR-19b, miR-20a and miR-92a in the 3′ UTR of DNMT-1 were identified. Using a luciferase system, transfection of miR-17, miR-19b, miR-20a or miR-92a into HEK-293 cells expressing the 3′-UTR WT DNMT-1-luc construct reduced cellular luciferase activity, an effect abrogated by mutating the DNMT-1 seed sequence (FIG. 38C). There was no change in DNMT-1 luciferase production in the presence of miR-19a and miR-18a, which do not have a predicted seed sequence in the 3′ UTR for DNMT-1.

5′-aza-2′-deoxycytidine Alters Gene Expression in Pulmonary Fibrosis

Using a murine model of pulmonary fibrosis, it was then determined whether 5′-aza-2′-deoxycytidine treatment altered miR-17˜92 DNA methylation, fibrotic gene expression and lung fibrosis. Similar to the pathological fibrotic pattern observed in human IPF, mice given bleomycin twice weekly by intraperitoneal injection for four weeks develop sub-pleural fibrosis of the lung. In this model, fibrosis appears in the lungs within two weeks of treatment.

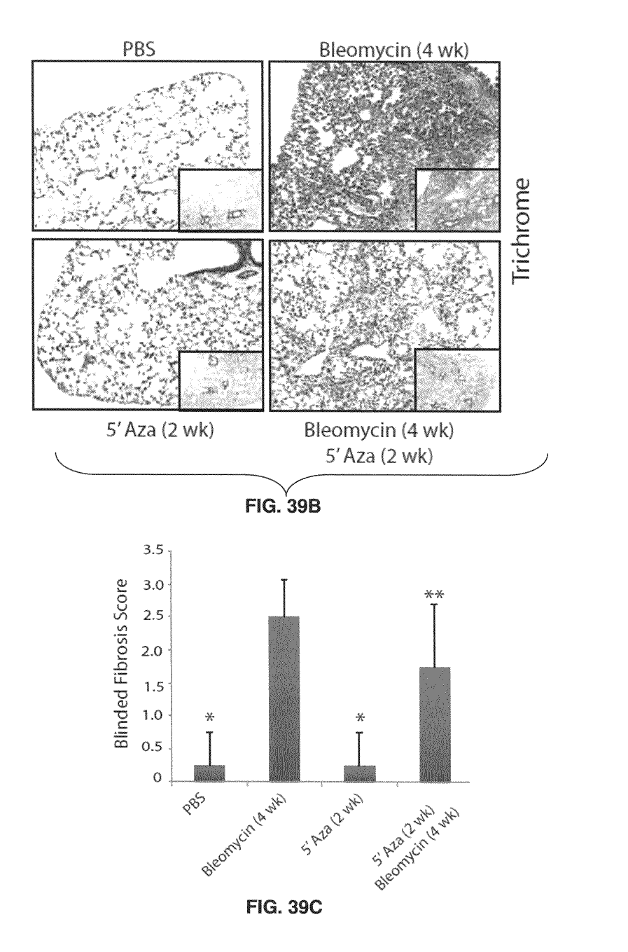

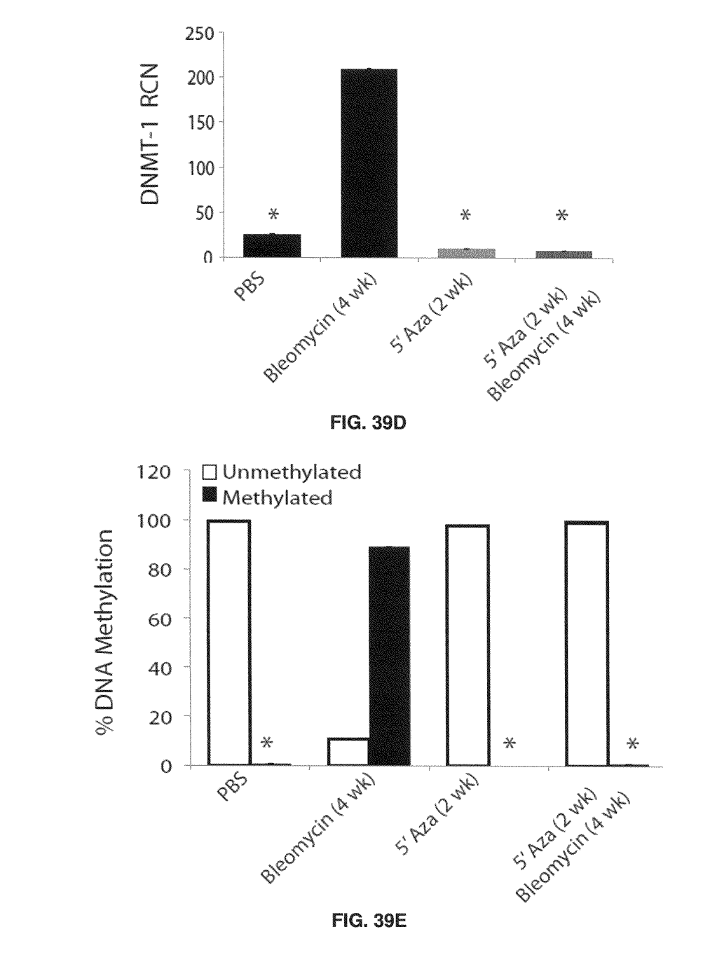

To assess the utility of demethylating agents in lung fibrosis, mice were treated for two weeks with intraperitoneal bleomycin injections prior to treatment with 5′-aza-2′-deoxycytidine or vehicle during the final two weeks of bleomycin treatment. As shown in FIG. 39A, repeated systemic bleomycin treatment reduced miR-17˜92 cluster expression in murine lung tissue, which was abrogated by 5′-aza-2′-deoxycytidine treatment, even when started two weeks after bleomycin injections started. Although not significant (p=0.1348), 5′-aza-2′-deoxycytidine treatment altered lung pathology after two weeks of bleomycin (FIG. 39B and FIG. 39C) and significantly reduced collagen, VEGF, CTGF and DNMT-1 gene expression (FIG. 39D and FIG. 39E).

It is to be noted that 5′-aza-2′-deoxycytidine enhanced the endogenous expression of the miR-1˜92 cluster leading to a reduction in the indicated genes in the lung tissue. Also, 5′-aza-2′-deoxycytidine abolished bleomycin-induced DNA methylation of the miR-17˜92 cluster compared to mice treated with bleomycin alone (FIG. 39F).

The miR-17˜92 cluster was decreased in lung tissue from IPF patients accompanied by enhanced DNA methylation of its promoter. Also, there was decreased expression of several members from the paralog miR-17˜92 clusters (miR-106a˜363 and miR-106b˜25),showing an unlikely compensatory role for these miRNAs. Moreover, the demethylating agent, 5′-aza-2′-deoxycytidine, increased the expression of the miR-17˜92 cluster.

In addition, it is to be noted that miR-17˜92 expression was reduced in lung tissue from patients with IPF, but not in COPD lung tissue.

Also, miRNAs contained in the miR-17˜92 cluster targeted numerous fibrotic genes and DNMTs, thus showing that IPF is a disease of aberrant DNA methylation. It is now shown herein that the miR-17˜92 cluster specifically targeted DNMT-1, and that introducing the miR-17˜92 cluster into IPF lung fibroblasts reduced promoter methylation of the cluster and fibrotic gene expression.

An inverse relationship between the miR-17˜92 cluster and DNMT-1 expression was observed in IPF lung tissue. Also, DNMT-1 is recruited to sites of DNA damage where it regulates epigenetic events, and a-SMA expression is inversely regulated by DNMT expression in fibroblasts and TGFf3-induced myofibrobast differentiation.

Further, a decreased expression of miRs-19b and -20a from human lung tissue from IPF patients was accompanied by enhanced a-SMA expression (FIG. 34 and FIG. 42). Using either the miR-17˜92 cluster or 5′-aza-2′-deoxycytidine reduced DNMT-1 expression in human cells and reduced actin expression.

Proteins that bind methylated DNA can either repress or activate gene expression these include methyl DNA binding proteins (MBD) 1 and MBD2 as well as methyl CpG binding protein (MeCP2). Mice lacking MeCP2 have decreased collagen deposition and myofibroblast differentiation in response to intra-tracheal injection of bleomycin. The miR-17˜92 cluster is now believed to target MBD2 and MeCP2 but not MBD1, and epigenetic regulation by the miR-17˜92 cluster is regulated by targeting multiple proteins.

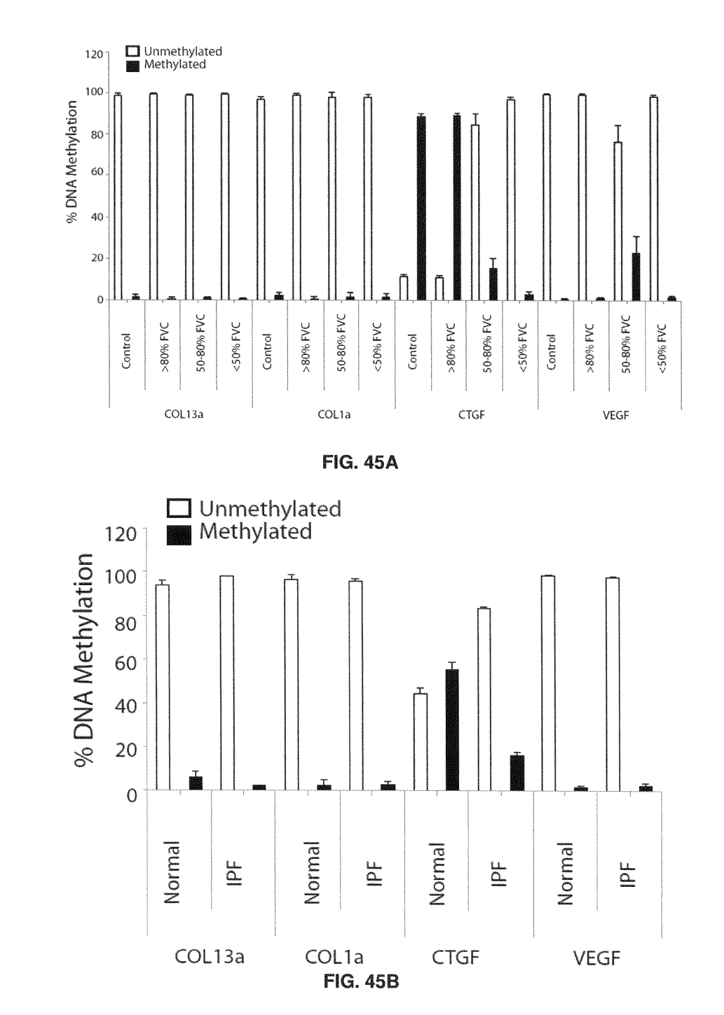

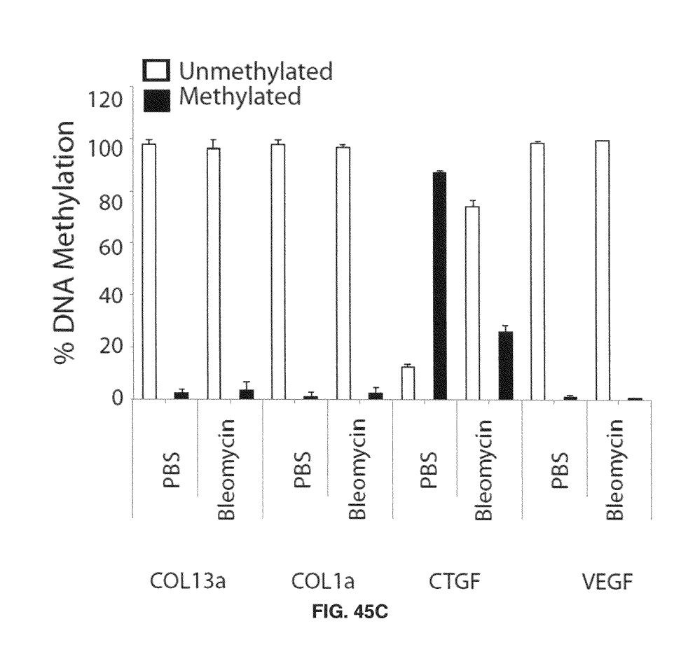

The chemotherapeutic drug 5′-aza-2′-deoxycytidine is associated with myelosuppression due to neutropenia, thrombocytopenia and anemia. It is now shown herein that 5′-aza-2′-deoxycytidine treatment of IPF fibroblasts increased the expression of the majority of miRNAs contained in the miR-17˜92 cluster and decreased the expression of fibrotic gene targets. It is also to be noted that the fibrotic genes examined herein have single or multiple CpG island(s). However, the promoters of the majority of these genes are unmethylated in control and IPF tissue (FIG. 45), thus showing a critical role for miRNAs in the regulation of their expression.

Also described herein is the utility of 5′-aza-2′-deoxycytidine in the treatment of pulmonary fibrosis. Treatment of bleomycin-treated mice with 5′-aza-2′-deoxycytidine after the initiation of fibrosis, acted to reduced fibrotic and DNMT-1 gene expression while restoring miR-17˜92 cluster expression. While lung pathology was not altered significantly, the 5′-aza-2′-deoxycytidine treatment does not mediate the breakdown of collagen already present, but prevents the deposition of additional collagen.

It is now shown herein that re-expression of the miR-17˜92 cluster using 5′-aza-2′-deoxycytidine leads to reduced fibrotic gene expression in vitro and in vivo, and that epigenetic modifying drugs have a therapeutic benefit for IPF.

Non-Limiting Examples of Uses

As described and exemplified herein particular miRNA are up- or down-regulated during tissue injury and/or inflammation.

As used herein interchangeably, a “miR gene product,” “microRNA,” “miR” or “miRNA” refers to the unprocessed or processed RNA transcript from a miR gene. As the miR gene products are not translated into protein, the term “miR gene products” does not include proteins. The unprocessed miR gene transcript is also called a “miR precursor,” and typically comprises an RNA transcript of about 70-100 nucleotides in length. The miR precursor can be processed by digestion with an RNAse (for example, Dicer, Argonaut, RNAse III (e.g., E. coli RNAse III)) into an active 19-25 nucleotide RNA molecule. This active 19-25 nucleotide RNA molecule is also called the “processed” miR gene transcript or “mature” miRNA.

The active 19-25 nucleotide RNA molecule can be obtained from the miR precursor through natural processing routes (e.g., using intact cells or cell lysates) or by synthetic processing routes (e.g., using isolated processing enzymes, such as isolated Dicer, Argonaut, or RNAse III). It is understood that the active 19-25 nucleotide RNA molecule can also be produced directly by biological or chemical synthesis, without having to be processed from the miR precursor. When a microRNA is referred to herein by name, the name corresponds to both the precursor and mature forms, unless otherwise indicated.

Also, in another aspect, there can be a library of sequence-specific miRNA inhibitors that can be used to inhibit sequentially or in combination the activities of one or more miRNAs in cells. The libraries of miRNA-specific reagents are useful to introduce or eliminate specific miRNAs or combinations of miRNAs to define the roles of miRNAs in cells.

The term “miRNA” is used according to its ordinary and plain meaning and refers to a microRNA molecule found in eukaryotes that is involved in RNA-based gene regulation. The term will be used to refer to the single-stranded RNA molecule processed from a precursor. Individual miRNAs have been identified and sequenced in different organisms, and they have been given names. Additionally, other miRNAs are known to those of skill in the art and can be readily implemented in embodiments described herein. The methods and compositions should not be limited to miRNAs identified in the application, as they are provided as examples, not necessarily as limitations of the invention.

In certain embodiments, short nucleic acid molecules that function as miRNAs or as inhibitors of miRNA in a cell are used. The term “short” generally refers to a length of a single polynucleotide that is 150 nucleotides or fewer. The nucleic acid molecules are “synthetic” in that the nucleic acid molecule is isolated and not identical in sequence (the entire sequence) and/or chemical structure to a naturally-occurring nucleic acid molecule, such as an endogenous precursor miRNA molecule. While in some embodiments, nucleic acids do not have an entire sequence that is identical to a sequence of a naturally-occurring nucleic acid, such molecules may encompass all or part of a naturally-occurring sequence. It is contemplated, however, that a synthetic nucleic acid administered to a cell may subsequently be modified or altered in the cell such that its structure or sequence is the same as non-synthetic or naturally occurring nucleic acid, such as a mature miRNA sequence. For example, a synthetic nucleic acid may have a sequence that differs from the sequence of a precursor miRNA, but that sequence may be altered once in a cell to be the same as an endogenous, processed miRNA.

The term “isolated” generally refers to the nucleic acid molecules that are initially separated from different (in terms of sequence or structure) and unwanted nucleic acid molecules such that a population of isolated nucleic acids is at least about 90% homogenous, and may be at least about 95, 96, 97, 98, 99, or 100% homogenous with respect to other polynucleotide molecules. In certain embodiments, a nucleic acid is isolated by virtue of it having been synthesized in vitro separate from endogenous nucleic acids in a cell. It is to be understood, however, that isolated nucleic acids may be subsequently mixed or pooled together. It is also to be understood that a “synthetic nucleic acid” can refer to a nucleic acid that does not have a chemical structure or sequence of a naturally occurring nucleic acid. Also, it is to be understood that the term “synthetic miRNA” can refer to a “synthetic nucleic acid” that functions in a cell or under physiological conditions as a naturally occurring miRNA.

In certain the embodiments, the nucleic acid molecule(s) need not be “synthetic.” In certain embodiments, a non-synthetic miRNA employed in methods and compositions may have the entire sequence and structure of a naturally occurring miRNA precursor or the mature miRNA. For example, non-synthetic miRNAs used in methods and compositions may not have one or more modified nucleotides or nucleotide analogs. In these embodiments, the non-synthetic miRNA may or may not be recombinantly produced. That is, certain embodiments discussed with respect to the use of synthetic miRNAs can be applied with respect to non-synthetic miRNAs, and vice versa.

It is also to be understood that the term “naturally occurring” generally refers to something found in an organism without any intervention by a person; it could refer to a naturally-occurring wildtype or mutant molecule. In some embodiments a synthetic miRNA molecule does not have the sequence of a naturally occurring miRNA molecule. In other embodiments, a synthetic miRNA molecule may have the sequence of a naturally occurring miRNA molecule, but the chemical structure of the molecule, particularly in the part unrelated specifically to the precise sequence (non-sequence chemical structure) differs from chemical structure of the naturally occurring miRNA molecule with that sequence. In some cases, the synthetic miRNA has both a sequence and non-sequence chemical structure that are not found in a naturally-occurring miRNA. Moreover, the sequence of the synthetic molecules will identify which miRNA is effectively being provided or inhibited; the endogenous miRNA can be referred to as the “corresponding miRNA.” Corresponding miRNA sequences that can be used include, but are not limited to, those sequences in found in publically available SEQ ID databases, synthetic nucleic acids having the same nucleotide sequence, as well as any other miRNA sequence, miRNA precursor sequence, or any sequence complementary thereof. In certain embodiments, the sequence is or is derived from a probe sequence that can be used to target the particular miRNA (or set of miRNAs) that can be used with that probe sequence.

Synthetic miRNA can be RNA or RNA analogs; in addition, mRNA inhibitors may be DNA or RNA, or analogs thereof miRNA and miRNA inhibitors of the invention are collectively referred to as “synthetic nucleic acids.” In some embodiments, there is a synthetic miRNA having a length of between 17 and 130 residues. In non-limiting examples, the synthetic miRNA molecules that can be at least, or are at most 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 residues in length, or any range derivable therein.

In certain embodiments, synthetic miRNA have a) an “miRNA region” whose sequence from 5′ to 3′ is identical to a mature miRNA sequence, and b) a “complementary region” whose sequence from 5′ to 3′ is between 60% and 100% complementary to the miRNA sequence. In certain embodiments, these synthetic miRNA are also isolated, as defined above. The term “miRNA region” refers to a region on the synthetic miRNA that is at least 90% identical to the entire sequence of a mature, naturally occurring miRNA sequence. In certain embodiments, the miRNA region is or is at least 90, 91, 92, 93, 94, 95, 96, 97, 98, 99, 99.1, 99.2, 99.3, 99.4, 99.5, 99.6, 99.7, 99.8, 99.9 or 100% identical to the sequence of a naturally-occurring miRNA.

The term “complementary region” refers to a region of a synthetic miRNA that is or is at least 60% complementary to the mature, naturally occurring miRNA sequence that the miRNA region is identical to. The complementary region is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein. With single polynucleotide sequences, there is a hairpin loop structure as a result of chemical bonding between the miRNA region and the complementary region. In other embodiments, the complementary region is on a different nucleic acid molecule than the miRNA region, in which case the complementary region is on the complementary strand and the miRNA region is on the active strand.

In other embodiments, there are synthetic nucleic acids that are miRNA “inhibitors.” An miRNA inhibitor can be between about 17 to 25 nucleotides in length and comprises a 5′ to 3′ sequence that is at least 90% complementary to the 5′ to 3′ sequence of a mature miRNA. In certain embodiments, an miRNA inhibitor molecule can be 17, 18, 19, 20, 21, 22, 23, 24, or 25 nucleotides in length, or any range derivable therein. Also, the miRNA inhibitor can have a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA.

Also, in certain embodiments, probe sequences for miRNAs can be used. It is to be understood that while probes may have more sequence than an miRNA inhibitor, one of skill in the art could use that portion of the probe sequence that is complementary to the sequence of a mature miRNA as the sequence for an miRNA inhibitor. It is also to be understood that portion of the probe sequence can be altered so that it is still 90% complementary to the sequence of a mature miRNA.

In some embodiments, a synthetic miRNA can contain one or more design elements. These design elements include, but are not limited to: i) a replacement group for the phosphate or hydroxyl of the nucleotide at the 5′ terminus of the complementary region; ii) one or more sugar modifications in the first or last 1 to 6 residues of the complementary region; or, iii) noncomplementarity between one or more nucleotides in the last 1 to 5 residues at the 3′ end of the complementary region and the corresponding nucleotides of the miRNA region.

In other embodiments, there can be a synthetic miRNA in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region. It is contemplated that synthetic miRNA of the invention have one or more of the replacement, sugar modification, or noncomplementarity designs. In certain cases, synthetic RNA molecules have two of them, while in others these molecules have all three designs in place.

In certain embodiments, the methods can comprise determining the level of at least one miR gene product in a sample from the subject and comparing the level of the miR gene product in the sample to a control. As used herein, a “subject” can be any mammal that has, or is suspected of having, such disorder. In a preferred embodiment, the subject is a human who has, or is suspected of having, such disorder.

The level of at least one miR gene product can be measured in cells of a biological sample obtained from the subject. The sample can be removed from the subject, and DNA can be extracted and isolated by standard techniques. For example, in certain embodiments, the sample can be obtained from the subject prior to initiation of radiotherapy, chemotherapy or other therapeutic treatment. A corresponding control sample, or a control reference sample (e.g., obtained from a population of control samples), can be obtained from unaffected samples of the subject, from a normal human individual or population of normal individuals, or from cultured cells corresponding to the majority of cells in the subject's sample. The control sample can then be processed along with the sample from the subject, so that the levels of miR gene product produced from a given miR gene in cells from the subject's sample can be compared to the corresponding miR gene product levels from cells of the control sample. Alternatively, a reference sample can be obtained and processed separately (e.g., at a different time) from the test sample and the level of a miR gene product produced from a given miR gene in cells from the test sample can be compared to the corresponding miR gene product level from the reference sample.

In one embodiment, the level of the at least one miR gene product in the test sample is greater than the level of the corresponding miR gene product in the control sample (i.e., expression of the miR gene product is “upregulated”). As used herein, expression of a miR gene product is “upregulated” when the amount of miR gene product in a sample from a subject is greater than the amount of the same gene product in a control (for example, a reference standard, a control cell sample, a control tissue sample).

In another embodiment, the level of the at least one miR gene product in the test sample is less than the level of the corresponding miR gene product in the control sample (i.e., expression of the miR gene product is “downregulated”). As used herein, expression of a miR gene is “downregulated” when the amount of miR gene product produced in a sample from a subject is less than the amount produced from the same gene in a control sample.

The relative miR gene expression in the control and normal samples can be determined with respect to one or more RNA expression standards. The standards can comprise, for example, a zero miR gene expression level, the miR gene expression level in a standard cell line, the miR gene expression level in unaffected samples of the subject, or the average level of miR gene expression previously obtained for a population of normal human controls (e.g., a control reference standard).

The level of the at least one miR gene product can be measured using a variety of techniques that are well known to those of skill in the art (e.g., quantitative or semi-quantitative RT-PCR, Northern blot analysis, solution hybridization detection). In a particular embodiment, the level of at least one miR gene product is measured by reverse transcribing RNA from a test sample obtained from the subject to provide a set of target oligodeoxynucleotides, hybridizing the target oligodeoxynucleotides to one or more miRNA-specific probe oligonucleotides (e.g., a microarray that comprises miRNA-specific probe oligonucleotides) to provide a hybridization profile for the test sample, and comparing the test sample hybridization profile to a hybridization profile generated from a control sample. An alteration in the signal of at least one miRNA in the test sample relative to the control sample is indicative of the subject either having, or being at risk for a particular disorder.

Also, a microarray can be prepared from gene-specific oligonucleotide probes generated from known miRNA sequences. The array may contain two different oligonucleotide probes for each miRNA, one containing the active, mature sequence and the other being specific for the precursor of the miRNA. The array may also contain controls, such as one or more mouse sequences differing from human orthologs by only a few bases, which can serve as controls for hybridization stringency conditions. tRNAs and other RNAs (e.g., rRNAs, mRNAs) from both species may also be printed on the microchip, providing an internal, relatively stable, positive control for specific hybridization. One or more appropriate controls for non-specific hybridization may also be included on the microchip. For this purpose, sequences are selected based upon the absence of any homology with any known miRNAs.

The microarray may be fabricated using techniques known in the art. For example, probe oligonucleotides of an appropriate length, e.g., 40 nucleotides, are 5′-amine modified at position C6 and printed using commercially available microarray systems, e.g., the GeneMachine OmniGrid™ 100 Microarrayer and Amersham CodeLink™ activated slides. Labeled cDNA oligomer corresponding to the target RNAs is prepared by reverse transcribing the target RNA with labeled primer. Following first strand synthesis, the RNA/DNA hybrids are denatured to degrade the RNA templates. The labeled target cDNAs thus prepared are then hybridized to the microarray chip under hybridizing conditions, e.g., 6×SSPE/30% formamide at 25° C. for 18 hours, followed by washing in 0.75×TNT at 37° C. for 40 minutes. At positions on the array where the immobilized probe DNA recognizes a complementary target cDNA in the sample, hybridization occurs. The labeled target cDNA marks the exact position on the array where binding occurs, allowing automatic detection and quantification. The output consists of a list of hybridization events, indicating the relative abundance of specific cDNA sequences, and therefore the relative abundance of the corresponding complementary miRs, in the patient sample. According to one embodiment, the labeled cDNA oligomer is a biotin-labeled cDNA, prepared from a biotin-labeled primer. The microarray is then processed by direct detection of the biotin-containing transcripts using, e.g., Streptavidin-Alexa647 conjugate, and scanned utilizing conventional scanning methods. Image intensities of each spot on the array are proportional to the abundance of the corresponding miR in the patient sample.

The use of the array has several advantages for miRNA expression detection. First, the global expression of several hundred genes can be identified in the same sample at one time point. Second, through careful design of the oligonucleotide probes, expression of both mature and precursor molecules can be identified. Third, in comparison with Northern blot analysis, the chip requires a small amount of RNA, and provides reproducible results using 2.5 μg of total RNA. The relatively limited number of miRNAs (a few hundred per species) allows the construction of a common microarray for several species, with distinct oligonucleotide probes for each. Such a tool allows for analysis of trans-species expression for each known miR under various conditions.

According to the expression profiling methods described herein, total RNA from a sample from a subject suspected of having a particular disorder can be quantitatively reverse transcribed to provide a set of labeled target oligodeoxynucleotides complementary to the RNA in the sample. The target oligodeoxynucleotides are then hybridized to a microarray comprising miRNA-specific probe oligonucleotides to provide a hybridization profile for the sample. The result is a hybridization profile for the sample representing the expression pattern of miRNA in the sample. The hybridization profile comprises the signal from the binding of the target oligodeoxynucleotides from the sample to the miRNA-specific probe oligonucleotides in the microarray. The profile may be recorded as the presence or absence of binding (signal vs. zero signal). More preferably, the profile recorded includes the intensity of the signal from each hybridization. The profile is compared to the hybridization profile generated from a normal control sample or reference sample. An alteration in the signal is indicative of the presence of, or propensity to develop, the particular disorder in the subject.

Other techniques for measuring miR gene expression are also within the skill in the art, and include various techniques for measuring rates of RNA transcription and degradation.

The invention also provides methods of diagnosing whether a subject has, or is at risk for developing, a particular disorder with an adverse prognosis. In this method, the level of at least one miR gene product, which is associated with an adverse prognosis in a particular disorder, is measured by reverse transcribing RNA from a test sample obtained from the subject to provide a set of target oligodeoxynucleotides. The target oligodeoxynucleotides are then hybridized to one or more miRNA-specific probe oligonucleotides (e.g., a microarray that comprises miRNA-specific probe oligonucleotides) to provide a hybridization profile for the test sample, and the test sample hybridization profile is compared to a hybridization profile generated from a control sample. An alteration in the signal of at least one miRNA in the test sample relative to the control sample is indicative of the subject either having, or being at risk for developing, a particular disorder with an adverse prognosis.

An “expression profile” or “hybridization profile” of a particular sample is essentially a fingerprint of the state of the sample; while two states may have any particular gene similarly expressed, the evaluation of a number of genes simultaneously allows the generation of a gene expression profile that is unique to the state of the cell. That is, normal samples may be distinguished from corresponding disorder-exhibiting samples. Within such disorder-exhibiting samples, different prognosis states (for example, good or poor long term survival prospects) may be determined. By comparing expression profiles of disorder-exhibiting samples in different states, information regarding which genes are important (including both upregulation and downregulation of genes) in each of these states is obtained.

The identification of sequences that are differentially expressed in disorder-exhibiting samples, as well as differential expression resulting in different prognostic outcomes, allows the use of this information in a number of ways. For example, a particular treatment regime may be evaluated (e.g., to determine whether a chemotherapeutic drug acts to improve the long-term prognosis in a particular subject). Similarly, diagnosis may be done or confirmed by comparing samples from a subject with known expression profiles. Furthermore, these gene expression profiles (or individual genes) allow screening of drug candidates that suppress the particular disorder expression profile or convert a poor prognosis profile to a better prognosis profile.

Alterations in the level of one or more miR gene products in cells can result in the deregulation of one or more intended targets for these miRs, which can lead to a particular disorder. Therefore, altering the level of the miR gene product (e.g., by decreasing the level of a miR that is upregulated in disorder-exhibiting cells, by increasing the level of a miR that is downregulated in disorder-exhibiting cells) may successfully treat the disorder.

Accordingly, the present invention encompasses methods of treating a disorder in a subject, wherein the expression of at least one miR gene product is regulated (e.g., down-regulated, upregulated) in the cells of the subject. In one embodiment, the level of at least one miR gene product in a test sample is greater than the level of the corresponding miR gene product in a control or reference sample. In another embodiment, the level of at least one miR gene product in a test sample is less than the level of the corresponding miR gene product in a control sample. When the at least one isolated miR gene product is downregulated in the test sample, the method comprises administering an effective amount of the at least one isolated miR gene product, or an isolated variant or biologically-active fragment thereof, such that proliferation of the disorder-exhibiting cells in the subject is inhibited.

For example, when a miR gene product is downregulated in a cell in a subject, administering an effective amount of an isolated miR gene product to the subject can inhibit proliferation of the cell. The isolated miR gene product that is administered to the subject can be identical to an endogenous wild-type miR gene product that is downregulated in the cell or it can be a variant or biologically-active fragment thereof.

As defined herein, a “variant” of a miR gene product refers to a miRNA that has less than 100% identity to a corresponding wild-type miR gene product and possesses one or more biological activities of the corresponding wild-type miR gene product. Examples of such biological activities include, but are not limited to, inhibition of expression of a target RNA molecule (e.g., inhibiting translation of a target RNA molecule, modulating the stability of a target RNA molecule, inhibiting processing of a target RNA molecule) and inhibition of a cellular process associated with cancer and/or a myeloproliferative disorder (e.g., cell differentiation, cell growth, cell death). These variants include species variants and variants that are the consequence of one or more mutations (e.g., a substitution, a deletion, an insertion) in a miR gene. In certain embodiments, the variant is at least about 70%, 75%, 80%, 85%, 90%, 95%, 98%, or 99% identical to a corresponding wild-type miR gene product.

As defined herein, a “biologically-active fragment” of a miR gene product refers to an RNA fragment of a miR gene product that possesses one or more biological activities of a corresponding wild-type miR gene product. As described above, examples of such biological activities include, but are not limited to, inhibition of expression of a target RNA molecule and inhibition of a cellular process associated with such disorder. In certain embodiments, the biologically-active fragment is at least about 5, 7, 10, 12, 15, or 17 nucleotides in length. In a particular embodiment, an isolated miR gene product can be administered to a subject in combination with one or more additional treatments. Suitable treatments include, but are not limited to, chemotherapy, radiation therapy and combinations thereof (e.g., chemoradiation).

When the at least one isolated miR gene product is upregulated in the cells, the method comprises administering to the subject an effective amount of a compound that inhibits expression of the at least one miR gene product, such that proliferation of the disorder-exhibiting cells is inhibited. Such compounds are referred to herein as miR gene expression-inhibition compounds. Examples of suitable miR gene expression-inhibition compounds include, but are not limited to, those described herein (e.g., double-stranded RNA, antisense nucleic acids and enzymatic RNA molecules).

As described herein, when the at least one isolated miR gene product is upregulated in cells, the method comprises administering to the subject an effective amount of at least one compound for inhibiting expression of the at least one miR gene product, such that proliferation of such cells is inhibited.

The terms “treat”, “treating” and “treatment”, as used herein, refer to ameliorating symptoms associated with a disease or condition, including preventing or delaying the onset of the disease symptoms, and/or lessening the severity or frequency of symptoms of the disease, disorder or condition. The terms “subject”, “patient” and “individual” are defined herein to include humans, animals, such as mammals, including, but not limited to, primates, cows, sheep, goats, horses, dogs, cats, rabbits, guinea pigs, rats, mice or other bovine, ovine, equine, canine, feline, rodent, or murine species. In a preferred embodiment, the animal is a human.

As used herein, an “isolated” miR gene product is one that is synthesized, or altered or removed from the natural state through human intervention. For example, a synthetic miR gene product, or a miR gene product partially or completely separated from the coexisting materials of its natural state, is considered to be “isolated.” An isolated miR gene product can exist in a substantially-purified form, or can exist in a cell into which the miR gene product has been delivered. Thus, a miR gene product that is deliberately delivered to, or expressed in, a cell is considered an “isolated” miR gene product. A miR gene product produced inside a cell from a miR precursor molecule is also considered to be an “isolated” molecule. According to the invention, the isolated miR gene products described herein can be used for the manufacture of a medicament for treating a subject (e.g., a human).

Isolated miR gene products can be obtained using a number of standard techniques. For example, the miR gene products can be chemically synthesized or recombinantly produced using methods known in the art. In one embodiment, miR gene products are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., U.S.A.), Pierce Chemical (part of Perbio Science, Rockford, Ill., U.S.A.), Glen Research (Sterling, Va., U.S.A.), ChemGenes (Ashland, Mass., U.S.A.) and Cruachem (Glasgow, UK).

Alternatively, the miR gene products can be expressed from recombinant circular or linear DNA plasmids using any suitable promoter. Non-limiting examples of suitable promoters for expressing RNA from a plasmid include, e.g., the U6 or H1 RNA pol III promoter sequences, or the cytomegalovirus promoters. Selection of other suitable promoters is within the skill in the art. The recombinant plasmids of the invention can also comprise inducible or regulatable promoters for expression of the miR gene products in cells (e.g., cells exhibiting a particular disorder).

The miR gene products that are expressed from recombinant plasmids can be isolated from cultured cell expression systems by standard techniques. The miR gene products that are expressed from recombinant plasmids can also be delivered to, and expressed directly in, cells.

The miR gene products can be expressed from a separate recombinant plasmid, or they can be expressed from the same recombinant plasmid. In one embodiment, the miR gene products are expressed as RNA precursor molecules from a single plasmid, and the precursor molecules are processed into the functional miR gene product by a suitable processing system, including, but not limited to, processing systems extant within a cell.

Selection of plasmids suitable for expressing the miR gene products, methods for inserting nucleic acid sequences into the plasmid to express the gene products, and methods of delivering the recombinant plasmid to the cells of interest are within the skill in the art. For example, in certain embodiments, a plasmid expressing the miR gene products can comprise a sequence encoding a miR precursor RNA under the control of the CMV intermediate-early promoter. As used herein, “under the control” of a promoter means that the nucleic acid sequences encoding the miR gene product are located 3′ of the promoter, so that the promoter can initiate transcription of the miR gene product coding sequences.

The miR gene products can also be expressed from recombinant viral vectors. It is contemplated that the miR gene products can be expressed from two separate recombinant viral vectors, or from the same viral vector. The RNA expressed from the recombinant viral vectors can either be isolated from cultured cell expression systems by standard techniques, or can be expressed directly in cells (e.g., cells exhibiting a particular disorder).

In other embodiments of the treatment methods of the invention, an effective amount of at least one compound that inhibits miR expression can be administered to the subject. As used herein, “inhibiting miR expression” means that the production of the precursor and/or active, mature form of miR gene product after treatment is less than the amount produced prior to treatment. One skilled in the art can readily determine whether miR expression has been inhibited in cells using, for example, the techniques for determining miR transcript level discussed herein Inhibition can occur at the level of gene expression (i.e., by inhibiting transcription of a miR gene encoding the miR gene product) or at the level of processing (e.g., by inhibiting processing of a miR precursor into a mature, active miR).

As used herein, an “effective amount” of a compound that inhibits miR expression is an amount sufficient to inhibit proliferation of cells in a subject suffering from a particular disorder. One skilled in the art can readily determine an effective amount of a miR expression-inhibiting compound to be administered to a given subject, by taking into account factors, such as the size and weight of the subject; the extent of disease penetration; the age, health and sex of the subject; the route of administration; and whether the administration is regional or systemic.

One skilled in the art can also readily determine an appropriate dosage regimen for administering a compound that inhibits miR expression to a given subject, as described herein. Suitable compounds for inhibiting miR gene expression include double-stranded RNA (such as short- or small-interfering RNA or “siRNA”), antisense nucleic acids, and enzymatic RNA molecules, such as ribozymes. Each of these compounds can be targeted to a given miR gene product and interfere with the expression (e.g., by inhibiting translation, by inducing cleavage and/or degradation) of the target miR gene product.

For example, expression of a given miR gene can be inhibited by inducing RNA interference of the miR gene with an isolated double-stranded RNA (“dsRNA”) molecule which has at least 90%, for example, at least 95%, at least 98%, at least 99%, or 100%, sequence homology with at least a portion of the miR gene product. In a particular embodiment, the dsRNA molecule is a “short or small interfering RNA” or “siRNA.”

In certain embodiments, administration of at least one miR gene product (and/or at least one compound for regulating miR expression) will affect the proliferation of cells (e.g., cells exhibiting a particular disorder) in a subject who has such disorder.

As used herein, to “alter the proliferation of cells exhibiting a particular disorder” can include one or more of: to kill the cells; to permanently or temporarily arrest or slow the growth of the cells; to reactive a desired gene expression in the cell; and, to modulate and/or reverse disease progression. For example, inhibition of cell proliferation can be inferred if the number of such cells in the subject remains constant or decreases after administration of the miR gene products or miR gene expression-regulating compounds. An inhibition of proliferation of cells exhibiting a particular disorder can also be inferred if the absolute number of such cells increases, but the rate of cell growth decreases.

A miR gene product or miR gene expression-regulating compound can also be administered to a subject by any suitable enteral or parenteral administration route. Suitable enteral administration routes for the present methods include, e.g., oral, rectal, or intranasal delivery. Suitable parenteral administration routes include, e.g., intravascular administration (e.g., intravenous bolus injection, intravenous infusion, intra-arterial bolus injection, intra-arterial infusion and catheter instillation into the vasculature); peri- and intra-tissue injection); subcutaneous injection or deposition, including subcutaneous infusion (such as by osmotic pumps); direct application to the tissue of interest, for example by a catheter or other placement device; and inhalation.

The miR gene products or miR gene expression-regulating compounds can be formulated as pharmaceutical compositions, sometimes called “medicaments,” prior to administering them to a subject, according to techniques known in the art. Accordingly, the invention encompasses pharmaceutical compositions for treating such disorder.

The present pharmaceutical compositions comprise at least one miR gene product or miR gene expression-regulating compound (or at least one nucleic acid comprising a sequence encoding the miR gene product or miR gene expression-regulating compound) (e.g., 0.1 to 90% by weight), or a physiologically-acceptable salt thereof, mixed with a pharmaceutically-acceptable carrier. In certain embodiments, the pharmaceutical composition of the invention additionally comprises one or more therapeutic agents. The pharmaceutical formulations of the invention can also comprise at least one miR gene product or miR gene expression-regulating compound (or at least one nucleic acid comprising a sequence encoding the miR gene product or miR gene expression-regulating compound), which are encapsulated by liposomes and a pharmaceutically-acceptable carrier.

Pharmaceutical compositions of the invention can also comprise conventional pharmaceutical excipients and/or additives. Suitable pharmaceutical excipients include stabilizers, antioxidants, osmolality adjusting agents, buffers, and pH adjusting agents. Suitable additives include, e.g., physiologically biocompatible buffers (e.g., tromethamine hydrochloride), additions of chelants (such as, for example, DTPA or DTPA-bisamide) or calcium chelate complexes (such as, for example, calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized

For solid pharmaceutical compositions of the invention, conventional nontoxic solid pharmaceutically-acceptable carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can comprise any of the carriers and excipients listed above and 10-95%, preferably 25%-75%, of the at least one miR gene product or miR gene expression-inhibition compound (or at least one nucleic acid comprising sequences encoding them). A pharmaceutical composition for aerosol (inhalational) administration can comprise 0.01-20% by weight, preferably 1%-10% by weight, of the at least one miR gene product or miR gene expression-regulating compound (or at least one nucleic acid comprising a sequence encoding the miR gene product or miR gene expression-regulating compound) encapsulated in a liposome as described above, and a propellant. A carrier can also be included as desired; e.g., lecithin for intranasal delivery.

In one embodiment, the method comprises providing a test agent to a cell and measuring the level of at least one miR gene product associated with an altered expression levels in such cells. An alteration in the level of the miR gene product in the cell, relative to a suitable control (e.g., the level of the miR gene product in a control cell), is indicative of the test agent being therapeutic agent. Non-limiting examples of suitable agents include, but are not limited to, drugs (e.g., small molecules, peptides), and biological macromolecules (e.g., proteins, nucleic acids). The agent can be produced recombinantly, synthetically, or it may be isolated (i.e., purified) from a natural source. Various methods for providing such agents to a cell (e.g., transfection) are well known in the art, and several of such methods are described hereinabove. Methods for detecting the expression of at least one miR gene product (e.g., Northern blotting, in situ hybridization, RT-PCR, expression profiling) are also well known in the art.

The relevant teachings of all publications cited herein that have not explicitly been incorporated by reference, are incorporated herein by reference in their entirety.

The miRs of interest are listed in public databases. In certain preferred embodiments, the public database can be a central repository provided by the Sanger Institute, microrna.sanger.ac.uk/sequences/to which miR sequences are submitted for naming and nomenclature assignment, as well as placement of the sequences in a database for archiving and for online retrieval via the world wide web.

Generally, the data collected on the sequences of miRs by the Sanger Institute include species, source, corresponding genomic sequences and genomic location (chromosomal coordinates), as well as full length transcription products and sequences for the mature fully processed miRNA (miRNA with a 5′ terminal phosphate group). Another database can be the GenBank database accessed through the National Center for Biotechnology Information (NCBI) website, maintained by the National Institutes of Health and the National Library of Medicine. These databases are fully incorporated herein by reference.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

What is claimed is:

1. A method of treating an inflammatory disorder in a subject having a decreased expression of DNA methyltransferase (DNMT), comprising administering to a subject an effective amount of at least one microRNA from the miR-17˜92 cluster, wherein the disorder has an decreased expression of a DNMT, as compared to a reference level.

2. The method of claim 1, wherein the miRNA is selected from the group consisting of: miR-17-5p, miR-17-3p, miR-18, miR-19a, miR-19b, miR-20a and miR-92.

3. The method of claim 1, wherein the miR comprises miR-19b and/or miR-20a.

4. The method of claim 1, further comprising administering one or more additional pharmaceutical DNMT inhibitor compositions.

5. The method of claim 4, wherein the additional DNMT inhibitor composition comprises 5′-aza-2′-deoxycytidine or an analog thereof.

6. The method of claim 1, wherein the inflammatory disorder is idiopathic pulmonary fibrosis (IPF), systemic sclerosis, pulmonary fibrosis, liver fibrosis, kidney fibrosis, uterine fibrosis, vascular fibrosis including peripheral arterial disease, or interventional therapy triggered fibrosis.

7. The method of claim 1, wherein the subject is a human.

8. The method of claim 1, wherein the administering: a) ameliorates the fibrosis; b) slows further progression of the fibrosis; c) halts further progression of the fibrosis; and/or d) reduces the fibrosis.

9. The method of claim 1, wherein the administration of the at least one miR decreases expression of DNMT in said idiopathic fibroblast cells without altering the phenotype of the non-idiopathic fibroblast cells.

10. A method of inhibiting an increase in DNTM levels induced by an inflammatory disorder over-expressing DNTM, comprising: contacting a human fibroblast cell over expressing human DNTM with an agent under conditions such that increases in DNTM in said fibroblast cell is inhibited, wherein said agent is an oligonucleotide that functions via RNA interference and the oligonucleotide sequence consists of at least one miR of the miR-17˜92 cluster.

11. A method of treating an inflammatory disorder in a subject having a decreased expression of DNA methyltransferase (DNMT), comprising administering to a subject an effective amount of 5′-aza-2′-deoxycytidine or an analog thereof.

12. A method of treating idiopathic pulmonary fibrosis, comprising administering to a patient in need thereof a therapeutically effective amount of microRNA selected from miR-17˜-92 cluster comprised of miR-17-5p, miR-17-3p, miR-18, miR-19a, miR-19b, miR-20a and miR-92 upregulator.

13. A method of treating an inflammatory disorder comprising:
administering a therapeutically effective amount of at least one DNA-methyltransferase (DNMT) inhibitor composition to a subject in need thereof; and
determining effectiveness of the DNMT inhibitor composition by measuring an increased expression of at least one microRNA selected from miR-17˜-92 cluster comprised of miR-17-5p, miR-17-3p, miR-18, miR-19a, miR-19b, miR-20a and miR-92.

14. The method of claim 13, wherein the inflammatory disorder is idiopathic pulmonary fibrosis (IPF).

15. A method of assessing the effectiveness of an DNA-methyltransferase inhibitor composition on the treatment of a fibrotic disease, comprising:
obtaining a biological sample from a subject before and after the treatment;
selecting at least one miRNA whose level of expression is increased or decreased in a cell that is being effectively treated with the DNA-methyltransferase inhibitor composition, as compared to the level of expression in a cell that is not being effectively treated;
measuring the level of the miRNA in the biological samples; and
determining if the miRNA is present at an increased or decreased level in the biological sample obtained after the treatment as compared to the biological sample obtained either before the treatment or in a cell that is not being effectively treated;
wherein an increased or decreased level of the miRNA indicates the effectiveness of the DNA-methyltransferase inhibitor composition in treating the disease.

16. The method of claim 16, wherein the result of the miRNA assessment is used to optimize the dosing regimen of the subject.

17. The method of claim 16, wherein the altered expression level is an increase in expression.

18. The method of claim 16, wherein the altered expression level is a decrease in expression.

19. The method of claim 16, wherein the miRNA is selected from the miR-17˜92 cluster, and further wherein the expression is increased after treatment.

20. The method of claim 16, wherein the treatment is a treatment for a fibrotic disorder.

21. The method of claim 16, wherein the treatment is an aerosol administration of the DNA-methyltransferase inhibitor composition.

22. The method of claim 16, wherein the subject is tested at a time interval selected from the group consisting of hourly, twice a day, daily, twice a week, weekly, twice a month, monthly, twice a year, yearly, and every other year.

23. A method of assessing the activity of a 5′-aza-2′-deoxycytidine type composition in a subject, comprising:
obtaining a biological sample from the subject before and after treatment of the subject; and
measuring the level of at least one miR selected from the miR-17˜92 cluster in the biological samples;
wherein an increased level of one or more of the miRNAs indicates the activity of the composition.

24. The method of claim 23, wherein the activity is the extent of the treatment by 5′-aza-2′-deoxycytidine.

25. The method of claim 23, wherein the extent of the treatment is a dose administered or length of subject's exposure to 5′-aza-2′-deoxycytidine.

26. The method of claim 23, wherein the treatment is a treatment for a fibrotic disease.

27. The method of claim 23, wherein the treatment is an aerosol administration of 5′-aza-2′-deoxycytidine.

28. The method of claim 23, wherein the subject is tested at a time interval selected from the group consisting of hourly, twice a day, daily, twice a week, weekly, twice a month, monthly, twice a year, yearly, and every other year.

29. A method of treating or delaying the onset or recurrence of a fibrotic associated disorder, wherein the disorder involves airway inflammation, fibrosis and excess mucus production, or at least one symptom thereof, the method comprising: administering an effective amount of: 5′-aza-2′-deoxycytidine and/or a miR-17˜92 gene product.

30. The method of claim 30, wherein the 5′-aza-2′-deoxycytidine is administered by inhalation.

31. A composition comprising a pharmacologically effective dose of a 5′-aza-2′-deoxycytidine and a pharmacologically effective dose of one or more miR-17˜92 gene products.

32. A composition according to claim 31, wherein the 5′-aza-2′-deoxycytidine and the one or more miR-17˜92 gene products are in dosage unit form.

33. A composition according to claim 31 wherein the composition is in the form of a, spray or aerosol.

34. A composition according to claim 31, wherein the ratio of 5′-aza-2′-deoxycytidine to one or more miRNA selected from miR-17˜92 gene products in the dosage form is in the range of 2:1 to 1:2.

35. A composition according to claim 31, wherein the ratio of 5′-aza-2′-deoxycytidine to one or more miRNA selected from miR-17˜92 gene products in the dosage form is 1:2.

36. A composition according to claim 31, further comprising a pharmaceutically acceptable carrier.
